SEQUENCE LISTING (95 sequences)

SEQ ID NO: 1 - protein, 57 aa, Escherichia coli
SEKKQPVDLGLLEEDDEFEEFPAEDWAGLDEDEDAHVWEDNWDDDNVEDDFSNQLRA

SEQ ID NO: 2 - protein, 31 aa, Escherichia coli
TVLLMILVCETPYGCYVLHQKGRMCSAFLCC

SEQ ID NO: 3 - protein, 31 aa, Escherichia coli
TVLLMILVAETPYGAYVLHQKGRMASAFLAA

SEQ ID NO: 4 - protein, 31 aa, Escherichia coli
TVLLMILVCETPYGCYVLHQKGRMCSAFLCA

SEQ ID NO: 5 - protein, 31 aa, Escherichia coli
TVLLMILVVETPYGVYVLHQKGRMVSAFLVV

SEQ ID NO: 6 - protein, 31 aa, Escherichia coli
TVLLMILVAETPYGAYVLHQKGRMVSAFLVV

SEQ ID NO: 7 - protein, 31 aa, Escherichia coli
TVLLMILVAETPYGVYVLHQKGRMASAFLVV

SEQ ID NO: 8 - protein, 31 aa, Escherichia coli
TVLLMILVAETPYGVYVLHQKGRMVSAFLAV

SEQ ID NO: 9 - protein, 30 aa, Escherichia coli
VLLMILVAETPYGVYVLHQKGRMVSAFLVA

SEQ ID NO: 10 - protein, 31 aa, Escherichia coli
TVLLMILVVETPYGAYVLHQKGRMASAFLVV

SEQ ID NO: 11 - protein, 31 aa, Escherichia coli
TVLLMILVVETPYGAYVLHQKGRMVSAFLAV

SEQ ID NO: 12 - protein, 31 aa, Escherichia coli
TVLLMILVVETPYGAYVLHQKGRMVSAFLVA

SEQ ID NO: 13 - protein, 31 aa, Escherichia coli
TVLLMILVVETPYGVYVLHQKGRMASAFLAV

SEQ ID NO: 14 - protein, 31 aa, Escherichia coli
TVLLMILVVETPYGVYVLHQKGRMASAFLVA

SEQ ID NO: 15 - protein, 31 aa, Escherichia coli
TVLLMILVVETPYGVYVLHQKGRMVSAFLAA

SEQ ID NO: 16 - protein, 31 aa, Escherichia coli
TVLLVILVAETPYGAYVLHQKGRVASAFLAA

SEQ ID NO: 17 - protein, 31 aa, Escherichia coli
GLCALCLMVRQPCFCLEIYMGVKHTCLYVST

SEQ ID NO: 18 - protein, 31 aa, Escherichia coli
SLLCVFPTIECCRYKMGLLYHCVACTQGMLV

SEQ ID NO: 19 - protein, 57 aa, Escherichia coli
SEFEDANKEQGEVPNDDVSDELPKLLDDDEDELRGALFFQWHDWEEAEDNDWDVEDA

SEQ ID NO: 20 - protein, 57 aa, Escherichia coli
SELDFWDNWKDRQADLPAASDDEDFENDLAEVDHEDPLVDGNEWDGDEVEFELEEQK

SEQ ID NO: 21 - protein, 57 aa, Escherichia coli
SDADEFDDDNSVWGWEDDEVEPNDAFQPLGDVRANEELDEKDHKLDEEADLQWEFEL

SEQ ID NO: 22 - protein, 57 aa, Escherichia coli
SQWAPLDHLESDEWGAFEDEDFNNGDVEELDPEERDDEDLFQKDVLVAKADDEEWDN

SEQ ID NO: 23 - protein, 57 aa, Escherichia coli
STKKQPVDLGLLETDSEFTEFPATSWAGLSESESAHVWTSNWDSSNVTSDFSNQLRA

SEQ ID NO: 24 - protein, 57 aa, Escherichia coli
STKKQPVDLGLLETDDEFEEFPATSWAGLSEDESAHVWESNWDDSNVTSDFSNQLRA

SEQ ID NO: 25 - protein, 57 aa, Escherichia coli
STKKQPVDLGLLEEDDEFEEFPAESWAGLDEDESAHVWEDNWDDDNVTSDFSNQLRA

SEQ ID NO: 26 - protein, 57 aa, Escherichia coli
SEKKQPVDLGLLEEDDEFEEFPAEDWAGLDEDESAHVWEDNWDDDNVTSDFSNQLRA

SEQ ID NO: 27 - protein, 57 aa, Escherichia coli
SEKKQPVDLGLLEEDDEFEEFPAEDWAGLDEDEDAHVWEDNWDDDNVTSDFSNQLRA

SEQ ID NO: 28 - protein, 88 aa, Escherichia coli
SEKKQPVDLGLLEEDDEFEEFPAEDWAGLDEDEDAHVWEDNWDDDNVEDDFSNQLRATVLLMILVCETPYGCYVLHQKGRMCSAFLCC

SEQ ID NO: 29 - protein, 88 aa, Escherichia coli
SEKKQPVDLGLLEEDDEFEEFPAEDWAGLDEDEDAHVWEDNWDDDNVEDDFSNQLRATVLLMILVAETPYGAYVLHQKGRMASAFLAA

SEQ ID NO: 30 - protein, 88 aa, Escherichia coli
SEKKQPVDLGLLEEDDEFEEFPAEDWAGLDEDEDAHVWEDNWDDDNVEDDFSNQLRATVLLMILVCETPYGCYVLHQKGRMCSAFLCA

SEQ ID NO: 31 - protein, 88 aa, Escherichia coli
SEKKQPVDLGLLEEDDEFEEFPAEDWAGLDEDEDAHVWEDNWDDDNVEDDFSNQLRATVLLMILVVETPYGVYVLHQKGRMVSAFLVV

SEQ ID NO: 32 - protein, 88 aa, Escherichia coli
SEKKQPVDLGLLEEDDEFEEFPAEDWAGLDEDEDAHVWEDNWDDDNVEDDFSNQLRATVLLMILVAETPYGAYVLHQKGRMVSAFLVV

SEQ ID NO: 33 - protein, 88 aa, Escherichia coli
SEKKQPVDLGLLEEDDEFEEFPAEDWAGLDEDEDAHVWEDNWDDDNVEDDFSNQLRATVLLMILVAETPYGVYVLHQKGRMASAFLVV

SEQ ID NO: 34 - protein, 88 aa, Escherichia coli
SEKKQPVDLGLLEEDDEFEEFPAEDWAGLDEDEDAHVWEDNWDDDNVEDDFSNQLRATVLLMILVAETPYGVYVLHQKGRMVSAFLAV

SEQ ID NO: 35 - protein, 88 aa, Escherichia coli
SEKKQPVDLGLLEEDDEFEEFPAEDWAGLDEDEDAHVWEDNWDDDNVEDDFSNQLRATVLLMILVAETPYGVYVLHQKGRMVSAFLVA

SEQ ID NO: 36 - protein, 88 aa, Escherichia coli
SEKKQPVDLGLLEEDDEFEEFPAEDWAGLDEDEDAHVWEDNWDDDNVEDDFSNQLRATVLLMILVVETPYGAYVLHQKGRMASAFLVV

SEQ ID NO: 37 - protein, 88 aa, Escherichia coli
SEKKQPVDLGLLEEDDEFEEFPAEDWAGLDEDEDAHVWEDNWDDDNVEDDFSNQLRATVLLMILVVETPYGAYVLHQKGRMVSAFLAV

SEQ ID NO: 38 - protein, 88 aa, Escherichia coli
SEKKQPVDLGLLEEDDEFEEFPAEDWAGLDEDEDAHVWEDNWDDDNVEDDFSNQLRATVLLMILVVETPYGAYVLHQKGRMVSAFLVA

SEQ ID NO: 39 - protein, 88 aa, Escherichia coli
SEKKQPVDLGLLEEDDEFEEFPAEDWAGLDEDEDAHVWEDNWDDDNVEDDFSNQLRATVLLMILVVETPYGVYVLHQKGRMASAFLAV

SEQ ID NO: 40 - protein, 88 aa, Escherichia coli
SEKKQPVDLGLLEEDDEFEEFPAEDWAGLDEDEDAHVWEDNWDDDNVEDDFSNQLRATVLLMILVVETPYGVYVLHQKGRMASAFLVA

SEQ ID NO: 41 - protein, 88 aa, Escherichia coli
SEKKQPVDLGLLEEDDEFEEFPAEDWAGLDEDEDAHVWEDNWDDDNVEDDFSNQLRATVLLMILVVETPYGVYVLHQKGRMVSAFLAA

SEQ ID NO: 42 - protein, 88 aa, Escherichia coli
SEKKQPVDLGLLEEDDEFEEFPAEDWAGLDEDEDAHVWEDNWDDDNVEDDFSNQLRAGLCALCLMVRQPCFCLEIYMGVKHTCLYVST

SEQ ID NO: 43 - protein, 88 aa, Escherichia coli
SEKKQPVDLGLLEEDDEFEEFPAEDWAGLDEDEDAHVWEDNWDDDNVEDDFSNQLRASLLCVFPTIECCRYKMGLLYHCVACTQGMLV

SEQ ID NO: 44 - protein, 88 aa, Escherichia coli
SEKKQPVDLGLLEEDDEFEEFPAEDWAGLDEDEDAHVWEDNWDDDNVEDDFSNQLRATVLLVILVAETPYGAYVLHQKGRVASAFLAA

SEQ ID NO: 45 - protein, 88 aa, Escherichia coli
SEFEDANKEQGEVPNDDVSDELPKLLDDDEDELRGALFFQWHDWEEAEDNDWDVEDATVLLMILVCETPYGCYVLHQKGRMCSAFLCC

SEQ ID NO: 46 - protein, 88 aa, Escherichia coli
SELDFWDNWKDRQADLPAASDDEDFENDLAEVDHEDPLVDGNEWDGDEVEFELEEQKTVLLMILVCETPYGCYVLHQKGRMCSAFLCC

SEQ ID NO: 47 - protein, 88 aa, Escherichia coli
SDADEFDDDNSVWGWEDDEVEPNDAFQPLGDVRANEELDEKDHKLDEEADLQWEFELTVLLMILVCETPYGCYVLHQKGRMCSAFLCC

SEQ ID NO: 48 - protein, 88 aa, Escherichia coli
SQWAPLDHLESDEWGAFEDEDFNNGDVEELDPEERDDEDLFQKDVLVAKADDEEWDNTVLLMILVCETPYGCYVLHQKGRMCSAFLCC

SEQ ID NO: 49 - protein, 88 aa, Escherichia coli
STKKQPVDLGLLETDSEFTEFPATSWAGLSESESAHVWTSNWDSSNVTSDFSNQLRATVLLMILVCETPYGCYVLHQKGRMCSAFLCC

SEQ ID NO: 50 - protein, 88 aa, Escherichia coli
STKKQPVDLGLLETDDEFEEFPATSWAGLSEDESAHVWESNWDDSNVTSDFSNQLRATVLLMILVCETPYGCYVLHQKGRMCSAFLCC

SEQ ID NO: 51 - protein, 88 aa, Escherichia coli
STKKQPVDLGLLEEDDEFEEFPAESWAGLDEDESAHVWEDNWDDDNVTSDFSNQLRATVLLMILVCETPYGCYVLHQKGRMCSAFLCC

SEQ ID NO: 52 - protein, 88 aa, Escherichia coli
SEKKQPVDLGLLEEDDEFEEFPAEDWAGLDEDESAHVWEDNWDDDNVTSDFSNQLRATVLLMILVCETPYGCYVLHQKGRMCSAFLCC

SEQ ID NO: 53 - protein, 88 aa, Escherichia coli
SEKKQPVDLGLLEEDDEFEEFPAEDWAGLDEDEDAHVWEDNWDDDNVTSDFSNQLRATVLLMILVCETPYGCYVLHQKGRMCSAFLCC

SEQ ID NO: 54 - protein, 83 aa, Escherichia coli
SEKKQPVDLGLLEEDDEFEEFPAEDWAGLDEDEDAHVWEDNWDDDNVTSDFSNQLRATVLLMILVCETPYGCYVLHQKGRMCS

SEQ ID NO: 55 - protein, 78 aa, Escherichia coli
SEKKQPVDLGLLEEDDEFEEFPAEDWAGLDEDEDAHVWEDNWDDDNVTSDFSNQLRATVLLMILVCETPYGCYVLHQK

SEQ ID NO: 56 - protein, 68 aa, Escherichia coli
SEKKQPVDLGLLEEDDEFEEFPAEDWAGLDEDEDAHVWEDNWDDDNVTSDFSNQLRATVLLMILVCET

SEQ ID NO: 57 - protein, 63 aa, Escherichia coli
SEKKQPVDLGLLEEDDEFEEFPAEDWAGLDEDEDAHVWEDNWDDDNVTSDFSNQLRATVLLMI

SEQ ID NO: 58 - protein, 57 aa, Escherichia coli; identical to SEQ ID NO: 27

SEQ ID NO: 59 - protein, 57 aa, Escherichia coli; identical to SEQ ID NO: 27

SEQ ID NO: 60 - protein, 57 aa, Escherichia coli; identical to SEQ ID NO: 27

SEQ ID NO: 61 - protein, 57 aa, Escherichia coli; identical to SEQ ID NO: 27

SEQ ID NO: 62 - protein, 69 aa, Escherichia coli
SEKKQPVDLGLLEEDDEFEEFPAEDWAGLDEDEDAHVWEDNWDDDNVEDDFSNQLRAELEKHGYKMETS

SEQ ID NO: 63 - protein, 89 aa, Escherichia coli
MSEKKQPVDLGLLEEDDEFEEFPAEDWAGLDEDEDAHVWEDNWDDDNVEDDFSNQLRATVLLMILVCETPYGCYVLHQKGRMCSAFLCC

SEQ ID NO: 64 - protein, 89 aa, Escherichia coli; identical to SEQ ID NO: 63

SEQ ID NO: 65 - protein, 89 aa, Escherichia coli
MSEKKQPVDLGLLEEDDEFEEFPAEDWAGLDEDEDAHVWEDNWDDDNVEDDFSNQLRVTVLLMILVCETLYGCYVLHQKGRMCSAFLCC

SEQ ID NO: 66 - protein, 89 aa, Escherichia coli
MSEKKQPVDLGLLEEDDEFEEFPAEDWAGLDEDEDAHVWEDNWDDDNVEDDFSNQLRATILLMILVCETPYGCYVLHQKGRMCSAFLCC

SEQ ID NO: 67 - protein, 89 aa, Escherichia coli
MSEKKQPVDLGLLEEDDEFEEFPAEDWAGLDEDEDAHVWEDNWDDDNVEDDFSNQLRATVLLMVLVCETPYGCYVLHQKERMCSAFLCC

SEQ ID NO: 68 - protein, 89 aa, Escherichia coli
MSEKKQPVDLGLLEEDDEFEEFPAEDWAGLDEDEDAHVWEDNWDDDNVEDDFSNQLRATVLLMIKVYETPYGCYILHQKGRMCSAFLCC

SEQ ID NO: 69 - protein, 89 aa, Escherichia coli; identical to SEQ ID NO: 68

SEQ ID NO: 70 - protein, 89 aa, Escherichia coli; identical to SEQ ID NO: 68

SEQ ID NO: 71 - protein, 89 aa, Escherichia coli
MSEKKQPVDLGLLEEDDEFEEFPAEDWAGLDEDEDAHVWEDNWDDDNVEDDFSNQLRATVLLMKKVYETPYGCYILHQKGRMCSAFLCC

SEQ ID NO: 72 - protein, 89 aa, Escherichia coli; identical to SEQ ID NO: 68

SEQ ID NO: 73 - protein, 89 aa, Escherichia coli; identical to SEQ ID NO: 68

SEQ ID NO: 74 - protein, 89 aa, Escherichia coli; identical to SEQ ID NO: 68

SEQ ID NO: 75 - protein, 74 aa, Escherichia coli
SEKKQPVDLGLLEEDDEFEEFPAEDWAGLDEDEDAHVWEDNWDDDNVEDDFSNQLRATVLLMILVAETPYGAYV

SEQ ID NO: 76 - protein, 57 aa, Escherichia coli; identical to SEQ ID NO: 19

SEQ ID NO: 77 - protein, 57 aa, Escherichia coli; identical to SEQ ID NO: 20

SEQ ID NO: 78 - protein, 57 aa, Escherichia coli; identical to SEQ ID NO: 21

SEQ ID NO: 79 - protein, 57 aa, Escherichia coli; identical to SEQ ID NO: 22

SEQ ID NO: 80 - protein, 57 aa, Escherichia coli; identical to SEQ ID NO: 23

SEQ ID NO: 81 - protein, 57 aa, Escherichia coli; identical to SEQ ID NO: 24

SEQ ID NO: 82 - protein, 57 aa, Escherichia coli; identical to SEQ ID NO: 25

SEQ ID NO: 83 - protein, 57 aa, Escherichia coli; identical to SEQ ID NO: 26

SEQ ID NO: 84 - protein, 31 aa, Escherichia coli
GLAALALMVRQPAFALEIYMGVKHTALYVST

SEQ ID NO: 85 - protein, 31 aa, Escherichia coli
SLLAVFPTIEAARYKMGLLYHAVAATQGMLV

SEQ ID NO: 86 - protein, 88 aa, Escherichia coli
SEFEDANKEQGEVPNDDVSDELPKLLDDDEDELRGALFFQWHDWEEAEDNDWDVEDATVLLMILVAETPYGAYVLHQKGRMASAFLAA

SEQ ID NO: 87 - protein, 88 aa, Escherichia coli
SELDFWDNWKDRQADLPAASDDEDFENDLAEVDHEDPLVDGNEWDGDEVEFELEEQKTVLLMILVAETPYGAYVLHQKGRMASAFLAA

SEQ ID NO: 88 - protein, 88 aa, Escherichia coli
SDADEFDDDNSVWGWEDDEVEPNDAFQPLGDVRANEELDEKDHKLDEEADLQWEFELTVLLMILVAETPYGAYVLHQKGRMASAFLAA

SEQ ID NO: 89 - protein, 88 aa, Escherichia coli
SQWAPLDHLESDEWGAFEDEDFNNGDVEELDPEERDDEDLFQKDVLVAKADDEEWDNTVLLMILVAETPYGAYVLHQKGRMASAFLAA

SEQ ID NO: 90 - protein, 88 aa, Escherichia coli
STKKQPVDLGLLETDSEFTEFPATSWAGLSESESAHVWTSNWDSSNVTSDFSNQLRATVLLMILVAETPYGAYVLHQKGRMASAFLAA

SEQ ID NO: 91 - protein, 88 aa, Escherichia coli
STKKQPVDLGLLETDDEFEEFPATSWAGLSEDESAHVWESNWDDSNVTSDFSNQLRATVLLMILVAETPYGAYVLHQKGRMASAFLAA

SEQ ID NO: 92 - protein, 88 aa, Escherichia coli
STKKQPVDLGLLEEDDEFEEFPAESWAGLDEDESAHVWEDNWDDDNVTSDFSNQLRATVLLMILVAETPYGAYVLHQKGRMASAFLAA

SEQ ID NO: 93 - protein, 88 aa, Escherichia coli
SEKKQPVDLGLLEEDDEFEEFPAEDWAGLDEDESAHVWEDNWDDDNVTSDFSNQLRATVLLMILVAETPYGAYVLHQKGRMASAFLAA

SEQ ID NO: 94 - protein, 88 aa, Escherichia coli
SEKKQPVDLGLLEEDDEFEEFPAEDWAGLDEDEDAHVWEDNWDDDNVEDDFSNQLRAGLAALALMVRQPAFALEIYMGVKHTALYVST

SEQ ID NO: 95 - protein, 88 aa, Escherichia coli
SEKKQPVDLGLLEEDDEFEEFPAEDWAGLDEDEDAHVWEDNWDDDNVEDDFSNQLRASLLAVFPTIEAARYKMGLLYHAVAATQGMLV

(19) United States
(12) Patent Application Publication
(10) Pub. No.: US 2024/0360183 A1
(43) Pub. Date: Oct. 31, 2024

(54) NOVEL POLYPEPTIDE

(71) Applicant: Shanghai Puyou Biomedical Co., Ltd., Shanghai (CN)

(72) Inventors: Yinghao Zhang, Shanghai (CN); Guirui Yan, Shanghai (CN); Yao Wang, Shanghai (CN); Wenhan Li, Shanghai (CN)

(21) Appl. No.: 18/605,501

(22) Filed: Mar. 14, 2024

Related U.S. Application Data
(63) Continuation of application No. PCT/CN2022/119040, filed on Sep. 15, 2022.

(30) Foreign Application Priority Data
Sep. 15, 2021 (WO) PCT/CN2021/118499

(51) Int. Cl. C07K 14/245; A61K 9/00; A61K 38/00; A61P 27/04

(52) U.S. Cl. CPC C07K 14/245; A61K 9/0048; A61P 27/04; A61K 38/00

(57) ABSTRACT

The present disclosure provides a novel polypeptide. Also provided is an ophthalmic composition for treating or preventing dry eye (DE) or DE associated disorders. Also provided is a method for treating or preventing DE or DE associated disorders, stimulating tears, stabilizing tear film or any combination thereof in a subject in need thereof, comprising administering to the subject an effective amount of the novel peptides. Also provided is a contact lenses care product comprising the novel peptides and the preparation method thereof.

### CROSS-REFERENCE

This application is a continuation of International Application No. PCT/CN2022/119040, filed on Sep. 15, 2022, which application claims priority to International Application No. PCT/CN2021/118499, filed on Sep. 15, 2021, each of which is herein incorporated by reference in its entirety.

### SEQUENCE LISTING

The instant application contains a Sequence Listing which has been submitted electronically in XML format and is hereby incorporated by reference in its entirety. Said XML copy, created on Mar. 13, 2024, is named 64982-710_301_SL.xml and is 88,557 bytes in size.

### BACKGROUND

The hydrophobicity and/or hydrophilicity property of a peptide is essential for its stability such as shelf-life, and biological activity including secondary structural configuration, peptide-protein interaction, kinetics of adsorption and desorption, cell membrane translocation and penetration, and the like. The hydrophobic or hydrophilic character is also called hydropathic character, hydropathicity, or hydropathy. Improving the hydropathy property of a peptide such as by replacing one or more amino acids with ones with opposite hydropathy character may sometime improve the stability or biological activity of the peptide.

Tears are essential for clear vision, and for maintaining health of eyes. With each blink of eyelid, tears spread across the front surface of the eye, provide lubrication, wash away foreign matter to keep the eye surface smooth and clear, and reduce the risk of infection. Dry eye (DE) is a condition when the eye does not produce adequate tears, or when the tears evaporate too quickly. It is one of the most common eye disorders and often a chronic problem, particularly in older adults, DE associated symptoms include dryness, burning, irritation, redness, discharge, easily fatigued eyes, and blurred vision. In some patients, without appropriate treatment, DE may further result in scarring and even inflammation of the eye.

### SUMMARY

The present disclosure provides novel peptides. Also provided is an ophthalmic formulation comprising the novel peptides for treating DE. Also provided is a method for treating or preventing DE or DE associated disorders, stimulating tears, stabilizing tear film or any combination thereof in a subject in need thereof, comprising administering to the subject an effective amount of the novel peptides. Also provided is a contact lenses care product comprising the novel peptides and the preparation method thereof.

In one aspect, provided is an artificial polypeptide of formula (I):

X—Y (I),
wherein X is a moiety comprising a sequence having at least 80% identity with SEQ ID NO. 1, and
Y is a moiety comprising 10 to 50 amino acids, at least 50% of which are selected from I, V, L, F, C, M, and A;
wherein Y comprises a total number of Cysteine (C) of less than 5.

In some embodiments, X comprises a sequence having at least 90% identity with SEQ ID NO. 1. In some embodiments, X comprises a sequence having at least 95% identity with SEQ ID NO. 1. In some embodiments, X comprises a sequence of SEQ ID NO. 1. In some embodiments, at least 50% amino acids of X are selected from R, K, N, D, Q, E, and H. In some embodiments, X comprises a sequence having at least 90% identity with any one selected from SEQ ID NOs. 81-83.

In some embodiments, Y comprises a total number of Cysteine (C) of less than 4. In some embodiments, Y comprises a total number of Cysteine (C) of less than 3. In some embodiments, Y comprises a total number of Cysteine (C) of less than 2. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs. 3-15, 58-61 and 84-85. In some embodiments, Y comprises a sequence having at least 90% identity with any one selected from SEQ ID NOs. 3-18, 58-61 and 84-85.

In some embodiments, the artificial polypeptide comprises a sequence having at least 90% identity with any one selected from SEQ ID NOs. 29-41, 54-57 and 91-95.

In another aspect, provided is an artificial polypeptide of formula (II):

X—Y (II),
wherein X is a moiety comprising a sequence having at least 80% identity with SEQ ID NO, 1, and
Y is a moiety comprising 10 to 50 amino acids, at least 50% of which are selected from I, V, L, F, C, M, and A;
wherein Y comprises a sequence having at most 90% identity with SEQ ID NO. 2.

In some embodiments, X comprises a sequence having at least 90% identity with SEQ ID NO. 1. In some embodiments, X comprises a sequence having at least 95% identity with SEQ ID NO. 1. In some embodiments, X comprises a sequence of SEQ ID NO. 1. In some embodiments, at least 50% amino acids of X are selected from R, K, N, D, Q, E, and H.

In some embodiments, Y comprises a sequence having at most 80% identity with SEQ ID NO. 2. In some embodiments, Y comprises a sequence having at most 70% identity with SEQ ID NO. 2. In some embodiments, Y comprises a sequence having at most 50% identity with SEQ ID NO. 2. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs. 16-18. In some embodiments, Y comprises a sequence having at least 95% identity with any one selected from SEQ ID NOs. 16-18.

In some embodiments, the artificial polypeptide comprises a sequence having at least 90% identity with SEQ ID NOs. 42-44.

In another aspect, provided is an artificial polypeptide of formula (III):

X—Y (III),
wherein X is a moiety comprising 40 to 65 amino acids, at least 50% of which are selected from H, R, K, D, Q, N and E; and
Y is a moiety comprising a sequence having at least 80% identity with SEQ ID NO. 2,
wherein a total number of H, R, K, D, Q, N and E in X is less than 33.

In some embodiments, Y comprises a sequence having at least 90% identity with SEQ ID NO. 2. In some embodiments, Y comprises a sequence having at least 95% identity with SEQ ID NO. 2. In some embodiments, Y comprises a sequence of SEQ ID NO. 2. In some embodiments, at least 50% amino acids of Y are selected from I, V, L, F, C, M, and A.

In some embodiments, the total number of H, R, K, D, Q, N and E in X is less than 30. In some embodiments, the total number of H, R, K, D, Q, N and E in X is less than 25. In some embodiments, the total number of H, R, K, D, Q, N and E in X is less than 20. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10. In some embodiments, X comprises a sequence having at least 90% identity with any one selected from SEQ ID NOs. 23-27. In some embodiments, X comprises a sequence having at least 95% identity with any one selected from SEQ ID NOs. 23-27.

In some embodiments, the artificial polypeptide comprises a sequence having at least 90% identity with SEQ ID NOs. 49-53.

In another aspect, provided is an artificial polypeptide of formula (IV):

X—Y (IV),
wherein X is a moiety comprising 40 to 65 amino acids, at least 50% of which are selected from H, R, K, D, Q, N and E; and
Y is a moiety comprising a sequence having at least 80% identity with SEQ ID NO. 2,
wherein X comprises a sequence having at most 90% identity with SEQ ID NO. 1.

In some embodiments, X comprises a sequence having at most 80% identity with SEQ ID NO. 1. In some embodiments, X comprises a sequence having at most 70% identity with SEQ ID NO. 1. In some embodiments, X comprises a sequence having at most 50% identity with SEQ ID NO. 1.

In some embodiments, Y comprises a sequence having at least 90% identity with SEQ ID NO. 2. In some embodiments, Y comprises a sequence having at least 95% identity with SEQ ID NO. 2. In some embodiments, Y comprises a sequence of SEQ ID NO. 2. In some embodiments, at least 50% amino acids of Y are selected from I, V, L, F, C, M, and A. In some embodiments, a total number of hydrophilic amino acids in X is more than 10. In some embodiments, a total number of hydrophilic amino acids in X is more than 15.

In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10. In some embodiments, X comprises a sequence having at least 90% identity with any one selected from SEQ ID NOs. 19-22 and 76-79. In some embodiments, X comprises a sequence having at least 95% identity with any one selected from SEQ ID NOs. 19-22 and 76-79.

In some embodiments, the artificial polypeptide comprises a sequence having at least 90% identity with SEQ ID NOs. 45-48 and 86-89.

In another aspect, provided is an artificial polypeptide of formula (V):

X—Y (V),
wherein X is a moiety comprising a sequence having at least 80% identity with SEQ ID NO, 1, and
Y is a moiety comprising 10 to 30 amino acids, wherein Y comprises a sequence having at least 10 continuous AAs of SEQ ID NO: 2.

In some embodiments, Y comprises 10 to 25 amino acids. In some embodiments, Y comprises 10 to 20 amino acids. In some embodiments, Y comprises 10 to 15 amino acids.

In some embodiments, X comprises a sequence having at least 90% identity with SEQ ID NO. 1. In some embodiments, X comprises a sequence having at least 95% identity with SEQ ID NO. 1. In some embodiments, X comprises a sequence of SEQ ID NO. 1.

In some embodiments, at least 50% amino acids of X are selected from R, K, N, D, Q, E, and H. In some embodiments, Y comprises a sequence having at most 80% identity with SEQ ID NO. 2. In some embodiments, Y comprises a sequence having at most 70% identity with SEQ ID NO. 2. In some embodiments, Y comprises a sequence having at most 60% identity with SEQ ID NO. 2. In some embodiments, Y comprises a sequence having at most 50% identity with SEQ ID NO. 2.

In some embodiments, the polypeptide comprises a sequence having at least 90% identity with SEQ ID NOs. 53-56. In some embodiments, the polypeptide comprises a sequence having at least 95% identity with SEQ ID NOs. 53-56. In some embodiments, the polypeptide comprises a sequence of any of SEQ ID NOs, 53-56.

In another aspect, provided is an artificial polypeptide of formula (VI):

X—Y (VI),
wherein X is a moiety comprising a mutant of the sequence SEQ ID NO. 1, characterized in that the mutant has at least 1, 2, 3, 4, 5, 6, 7, 8, or 9 D in SEQ ID NO. 1 mutated to S, and/or at least 1, 2, 3, 4, 5, or 6 E in SEQ ID NO. 1 mutated to T; and
Y is a moiety comprising a mutant of the sequence SEQ ID NO. 2, characterized in that the mutant has at least 1, 2, 3, 4, or 5 C in SEQ ID NO. 2 mutated to A.

In some embodiments, X comprises a sequence having at least 70% identity with SEQ ID NO. 1. In some embodiments, X comprises a sequence having at most 95% identity with SEQ ID NO. 1. In some embodiments, a total number of R, K, T, A, N, Q, D, E, S, and G in X is more than 30. In some embodiments, a total number of W, Y, F, M, L, I, and V in X is no more than 20. In some embodiments, X comprises a sequence having at least 80% identity with any one selected from SEQ ID NOs. 80-83. In some embodiments, X comprises a sequence that is any one selected from SEQ ID NOs. 80-83. In some embodiments, X is a sequence that is any one selected from SEQ ID NOs. 80-83.

In some embodiments, Y comprises a sequence having at least 80% identity with SEQ ID NO. 2. In some embodiments, Y comprises a sequence having at most 95% identity with SEQ ID NO. 2. In some embodiments, at least 50% amino acids of Y are selected from I, V, L, F, C, M, and A. In some embodiments, a total number of R, K, T, A, N, Q, D, E, S, and G in Y is more than 10. In some embodiments, a total number of W, Y, F, M, L, I, and V in Y is no more than 20. In some embodiments, Y comprises a sequence having at least 80% identity with SEQ ID NO. 3. In some embodiments, Y comprises a sequence of SEQ ID NO. 3. In some embodiments, Y is a sequence of SEQ ID NO. 3.

In some embodiments, the artificial polypeptide comprises a sequence having at least 80% identity with any one selected from SEQ ID NOs. 90-93. In some embodiments, the artificial polypeptide comprises a sequence that is any one selected from SEQ ID NOs. 90-93. In some embodiments, the artificial polypeptide is a sequence that is any one selected from SEQ ID NOs. 90-93.

In another aspect, provided is mutant of artificial polypeptide of formula (I),

X—Y (I),
wherein X is a moiety comprising a sequence having at least 80% identity with SEQ ID NO, 1,
Y is a moiety comprising 10 to 50 amino acids, at least 50% of which are selected from I, V, L, F, C, M, and A,
Y comprises a total number of Cysteine (C) of less than 5,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO. 1.

In some embodiments, Y comprises a total number of Cysteine (C) of less than 4. In some embodiments, Y comprises a total number of Cysteine (C) of less than 3. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs. 3-15, 58-61 and 84-85. In some embodiments, Y comprises a sequence having at least 90% identity with anyone selected from SEQ ID NOs. 3-18, 58-61 and 84-85.

In another aspect, provided is mutant of artificial polypeptide of formula (II),

X—Y (II),
wherein X is a moiety comprising a sequence having at least 80% identity with SEQ ID NO, 1,
Y is a moiety comprising 10 to 50 amino acids, at least 50% of which are selected from I, V, L, F, C, M, and A,
Y comprises a sequence having at most 90% identity with SEQ ID NO. 2,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO. 1.

In some embodiments, Y comprises a sequence having at most 80% identity with SEQ ID NO. 2. In some embodiments, Y comprises a sequence having at most 70% identity with SEQ ID NO. 2. In some embodiments, a total number of hydrophobic amino acids in Y is more than 8. In some embodiments, the total number of hydrophobic amino acids in Y is more than 12. In some embodiments, the total number of hydrophobic amino acids in Y is more than 15. In some embodiments, a total number of hydrophilic amino acids in Y is no more than 5. In some embodiments, Y comprises a sequence having at least 90% identity with any one selected from SEQ ID NOs. 16-18. In some embodiments, Y comprises a sequence having at least 95% identity with any one selected from SEQ ID NOs. 16-18.

In another aspect, provided is a mutant of artificial polypeptide of formula (III),

X—Y (III),
wherein X is a moiety comprising 40 to 65 amino acids, at least 50% of which are selected from H, R, K, D, Q, N and E,
Y is a moiety comprising a sequence having at least 80% identity with SEQ ID NO. 2, a total number of H, R, K, D, Q, N and E in X is less than 33,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO. 1. In some embodiments, the total number of H, R, K, D, Q, N and E in X is less than 30. In some embodiments, the total number of H, R, K, D, Q, N and E in X is less than 25. In some embodiments, the total number of H, R, K, D, Q, N and E in X is less than 20. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10.

In some embodiments, Y comprises a sequence having at least 90% identity with SEQ ID NO. 2. In some embodiments, Y comprises a sequence of SEQ ID NO. 2. In some embodiments, at least 50% amino acids of Y are selected from I, V, L, F, C, M, and A.

In another aspect, provided is a mutant of artificial polypeptide of formula (IV),

X—Y (IV),
wherein X is a moiety comprising 40 to 65 amino acids, at least 50% of which are selected from H, R, K, D, Q, N and E,
Y is a moiety comprising a sequence having at least 80% identity with SEQ ID NO. 2,
X comprises a sequence having at most 90% identity with SEQ ID NO. 1,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO. 1. In some embodiments, a total number of hydrophilic amino acids in X is more than 10. In some embodiments, a total number of hydrophilic amino acids in X is more than 15. In some embodiments, a total number of hydrophilic amino acids in X is more than 20. In some embodiments, a total number of hydrophilic amino acids in X is more than 25. In some embodiments, a total number of hydrophobic amino acids in X is no more than 15. In some embodiments, the total number of hydrophobic amino acids in X is no more than 10.

In some embodiments, Y comprises a sequence having at least 90% identity with SEQ ID NO. 2. In some embodiments, Y comprises a sequence of SEQ ID NO. 2. In some embodiments, at least 50% amino acids of Y are selected from I, V, L, F, C, M, and A.

In another aspect, provided is a mutant of artificial polypeptide of formula (V),

X—Y (V),
wherein X is a moiety comprising a sequence having at least 80% identity with SEQ ID NO, 1,
Y is a moiety comprising 10 to 30 amino acids, wherein Y comprises a sequence having at least 10 continuous AAs of SEQ ID NO: 2,
characterized in that the mutant has at least 1, 2, 3, 4, 5, 6, 7, 8, or 9 D in X mutated to S, and/or at least 1, 2, 3, 4, 5, or 6 E in X mutated to T.

In some embodiments, at least 20% amino acids of X are selected from R, K, N, D, Q, E, and H. In some embodiments, at least 30% amino acids of X are selected from R, K, N, D, Q, E, and H. In some embodiments, at most 90% amino acids of X are selected from R, K, N, D, Q, E, and H. In some embodiments, at most 80% amino acids of X are selected from R, K, N, D, Q, E, and H. In some embodiments, X comprises a sequence having at least 70% identity with SEQ ID NO. 1.

In some embodiments, Y comprises 10 to 25 amino acids. In some embodiments, Y comprises 10 to 20 amino acids. In some embodiments, Y comprises 10 to 15 amino acids. In some embodiments, Y comprises a sequence having at most 80% identity with SEQ ID NO. 2. In some embodiments, Y comprises a sequence having at most 70% identity with SEQ ID NO. 2. In some embodiments, Y comprises a sequence having at most 50% identity with SEQ ID NO. 2.

In another aspect, provided is an ophthalmic formulation for treating or preventing dry eye (DE) or DE associated disorders, stimulating tears, stabilizing tear film or any combination thereof, wherein the ophthalmic formulation comprises an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof.

In some embodiments, the DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia. Stevens-Johnson syndrome, ocular pemphigoid blepharitis marginal, eyelid-closure failure, corneal ulcer, blepharitis and eye redness.

In some embodiments, the polypeptide of the present disclosure (e.g., an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof) has a concentration from about 0.1 μM to about 100 μM. In some embodiments, the polypeptide has a concentration from about 0.1 μM to about 50 μM. In some embodiments, the polypeptide has a concentration from about 1 μM to about 5 μM. In some embodiments, the ophthalmic composition further comprises one or more pharmaceutically acceptable excipients. In some embodiments, the pharmaceutically acceptable excipient comprises stabilizer, buffer, preservative, tonicity agent, antioxidant, emulsifier, and viscosity-enhancing agent. In some embodiments, the ophthalmic composition is formulated as a solution, a gel, an ointment, a suspension, a semi-liquid, a semi-solid gel, a foam gel, a cream, a contact lens solution, or an eyewash. In some embodiments, the ophthalmic composition is formulated as an eye drop solution. In some embodiments, the ophthalmic composition is formulated for topical, subconjunctival, retrobulbar, periocular, subretinal, suprachoroidal, or intraocular administration.

In another aspect, provided is method for treating or preventing dry eye (DE) or DE associated disorders, stimulating tears, stabilizing tear film or any combination thereof in a subject in need thereof, comprising administering to the subject an effective amount of an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof.

In some embodiments, the artificial polypeptide is a polypeptide of any of SEQ ID NO. 28, SEQ ID NOs, 57 and 62-74 or a fragment or variant thereof. In some embodiments, the artificial polypeptide is a polypeptide of any of SEQ ID NO. 28, SEQ ID NOs. 57 and 62-74. In some embodiments, the artificial polypeptide is a polypeptide of any of SEQ ID NO. 28 and 62-74. In some embodiments, the artificial polypeptide is a polypeptide of SEQ ID NO. 28.

In some embodiments, the DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia. Stevens-Johnson syndrome, ocular pemphigoid blepharitis marginal, eyelid-closure failure, corneal ulcer, blepharitis and eye redness.

In another aspect, provided is an artificial polypeptide of formula (I), (II), (III), (IV), (V) or (VI), or a mutant of artificial polypeptide of (I), (II), (III), (IV) or (V), for use in therapy.

In another aspect, provided is an artificial polypeptide of formula (I), (II), (III), (IV), (V) or (VI), or a mutant of artificial polypeptide of (I), (II), (III), (IV) or (V), for use in treating or preventing dry eye (DE) or DE associated disorders, stimulating tears, stabilizing tear film or any combination thereof in a subject in need thereof.

In another aspect, provided is a polypeptide having at least 80% identity with any one selected from SEQ ID NO. 28, SEQ ID NOs. 57 and 62-74 or a fragment or variant thereof, for use in treating or preventing dry eye (DE) or DE associated disorders, stimulating tears, stabilizing tear film or any combination thereof in a subject in need thereof.

In some embodiments, the DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia. Stevens-Johnson syndrome, ocular pemphigoid blepharitis marginal, eyelid-closure failure, corneal ulcer, blepharitis and eye redness.

In another aspect, provided is use of an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof, for the preparation of a medicament for treating or preventing dry eye (DE) or DE associated disorders, stimulating tears, stabilizing tear film or any combination thereof in a subject in need thereof.

In some embodiments, the DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia. Stevens-Johnson syndrome, ocular pemphigoid blepharitis marginal, eyelid-closure failure, corneal ulcer, blepharitis and eye redness.

In another aspect, provided is a contact lenses care product, wherein the contact lenses care product comprises an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO, 28, SEQ ID NOs. 57 and 62-74 or a fragment or variant thereof.

In some embodiments, the contact lenses care product further comprises one or more acceptable ingredients for contact lenses care product. In some embodiments, the one or more acceptable ingredients for contact lenses care product are selected from the group consisting of inorganic salts, boric acid, surfactants, moisturizers, preservatives, and solvents. In some embodiments, the inorganic salts comprise sodium chloride, potassium chloride, sodium dihydrogen phosphate, disodium hydrogen phosphate, sodium carbonate, sodium borate, or any combination thereof. In some embodiments, the moisturizers comprise hyaluronic acid or a salt thereof. In some embodiments, the solvents comprise water. In some embodiments, the contact lenses care product is a solution for storing, protecting and/or cleansing contact lenses.

In another aspect, provided is a method for preparing a contact lenses care product, comprising the step of combining an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof with one or more acceptable ingredients for contact lenses care product. In some embodiments, the one or more acceptable ingredients for contact lenses care product are selected from the group consisting of inorganic salts, boric acid, surfactants, moisturizers, preservatives, and solvents. In some embodiments, the inorganic salts sodium chloride, potassium chloride, sodium dihydrogen phosphate, disodium hydrogen phosphate, sodium carbonate, sodium borate, or any combination thereof. In some embodiments, the solvents comprise water. In some embodiments, the contact lenses care product is a solution for storing, protecting and/or cleansing contact lenses.

In another aspect, provided is use of an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof for the preparation of a contact lenses care product.

In some embodiments, the contact lenses care product further comprises one or more acceptable ingredients for contact lenses care product. In some embodiments, the one or more acceptable ingredients for contact lenses care product are selected from the group consisting of inorganic salts, boric acid, surfactants, moisturizers, preservatives, and solvents. In some embodiments, the inorganic salts comprise sodium chloride, potassium chloride, sodium dihydrogen phosphate, disodium hydrogen phosphate, sodium carbonate, sodium borate, or any combination thereof. In some embodiments, the moisturizers comprise hyaluronic acid or a salt thereof. In some embodiments, the solvents comprise water. In some embodiments, the contact lenses care product is a solution for storing, protecting and/or cleansing contact lenses.

Additional aspects and advantages of the present disclosure will become readily apparent to those skilled in this art from the following detailed description, wherein only illustrative embodiments of the present disclosure are shown and described. As will be realized, the present disclosure is capable of other and different embodiments, and its several details are capable of modifications in various obvious respects, all without departing from the disclosure. Accordingly, the drawings and description are to be regarded as illustrative in nature, and not as restrictive.

### INCORPORATION BY REFERENCE

All publications, patents, and patent applications mentioned in this specification are herein incorporated by reference to the same extent as if each individual publication, patent, or patent application was specifically and individually indicated to be incorporated by reference.

### BRIEF DESCRIPTION OF THE DRAWINGS

Various features of this disclosure are set forth with particularity in the appended claims. A better understanding of the features and advantages of the present disclosure will be obtained by reference to the following detailed description that sets forth illustrative embodiments, in which the principles of the invention are utilized, and the accompanying drawings of which:

FIG. 1 illustrates effects of the polypeptides of the present disclosure on corneal fluorescein staining (CFS) scores of DE eyes.

FIG. 2 illustrates effects of the polypeptides of the present disclosure on tear break-up time (TBUT) of DE eyes.

FIG. 3A-3B and FIG. 4A-4B illustrates effects of the polypeptides of the present disclosure on acute severe DE eyes, FIG. 3A-3B illustrates CFS scores of severe DE eyes with the treatment of placebo and the polypeptides of the present disclosure, and FIG. 4A-4B illustrates TBUT of acute severe DE eyes with the treatment of placebo and the polypeptide of the present disclosure.

FIG. 5 illustrates effects of the polypeptides of the present disclosure on inhibiting thermal BSA aggregation.

FIG. 6 illustrates effects of the polypeptides of the present disclosure on oxLDL uptake.

FIG. 7 illustrates cell binding capacity of the polypeptides of the present disclosure.

FIG. 8 illustrates tear secretion and CFS score before and after administration of the polypeptides of the present disclosure.

The patent or application file contains at least one drawing executed in color. Copies of this patent or patent application publication with color drawings will be provided by the Office upon request and payment of the necessary fee.

### DETAILED DESCRIPTION

### Definition

As used herein, the following terms have the meanings ascribed to them unless specified otherwise.

As used in the specification and claims, the singular forms “a,” “an,” and “the” include plural references unless the context clearly dictates otherwise. For example, the term “a cell” includes a plurality of cells, including mixtures thereof.

As used herein, the terms “comprise”, “include”, “contain” and variations thereof are intended to mean open-ended transitional phrases that do not exclude the possibilities of additional substances or methods. When such terms are used to describe a certain pharmaceutical composition, formulation, kit, use or method of the present disclosure, it also encompasses the situation that the pharmaceutical composition, formulation, kit, use or method consists of the recited substances or methods. For instance, the expression “the solvents comprise water” also includes the situation wherein the solvents are consisting of water, i.e., the solvents contain water exclusively. In the context of this disclosure, the term “consisting of” is intended to mean a close-ended transitional phrase, which excludes the possibilities of additional substances or methods.

As used herein, ranges as recited in this disclosure are intended to explicitly disclose each of the endpoints of the range and each integer included in the range, unless otherwise indicated. For example, “X is a moiety comprising 40 to 65 amino acids” means that the number of amino acids in the moiety of X can be 40, 41, 42, 43, 44, 45, 46, 47, 48, 49, 50, 51, 52, 53, 54, 55, 56, 57, 58, 59, 60, 61, 62, 63, 64 or 65. For another example, “Y is a moiety comprising 10 to 50 amino acids” means that that the number of amino acids in moiety of Y can be 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49 or 50. Additionally, any sub-ranges consisting of these integers are intended to be included within the scope of this disclosure. Accordingly, “X is a moiety comprising 40 to 65 amino acids” is regarded as explicitly disclosing the sub-ranges such as “X is a moiety comprising 41 to 64 amino acids”, “X is a moiety comprising 42 to 63 amino acids”, “X is a moiety comprising 43 to 62 amino acids” . . . , etc.

The term “about” or “approximately” herein means within an acceptable error range of the particular value as determined by one of ordinary skill in the art, which will depend in part on how the value is measured or determined, i.e., the limitations of the measurement system. For example, “about” can mean within 1 or more than 1 standard deviation, per the practice in the art. Alternatively, “about” can mean a range of up to 20%, up to 10%, up to 5%, or up to 1% of a given value. Where particular values are described in the application and claims, unless otherwise stated, the term “about” or “approximately” meaning within an acceptable error range for the particular value should be assumed.

The terms “polypeptide,” “peptide,” and “protein” are used interchangeably herein to refer to polymers of amino acids of any length. The polymer may be linear or branched, it may comprise modified amino acids, and it may be interrupted by non-amino acids. The terms also encompass an amino acid polymer that has been modified, for example, by disulfide bond formation, glycosylation, lipidation, acetylation, phosphorylation, or any other manipulation, such as conjugation with a labeling component.

The term “fragment” as used herein, when applied to a protein, refers to a truncated form of a native biologically active protein that may or may not retain at least a portion of the therapeutic and/or biological activity.

The term “variant” as used herein, when applied to a protein, refers to a protein with sequence homology to the native biologically active protein that retains at least a portion of the therapeutic and/or biological activity of the biologically active protein. For example, a variant protein may share at least 70%, 75%, 80%, 85%, 90%, 95%, 96%, 97%, 98% or 99% amino acid sequence identity as compared to the reference biologically active protein.

As used herein the term “amino acid” refers to either natural and/or unnatural or synthetic amino acids, including but not limited to glycine and both the D or L optical isomers, and amino acid analogs and peptidomimetics. Standard single or three letter codes are used to designate amino acids.

The term “natural L-amino acid” means the L optical isomer forms of glycine (G), proline (P), alanine (A), valine (V), leucine (L), isoleucine (I), methionine (M), cysteine (C), phenylalanine (F), tyrosine (Y), tryptophan (W), histidine (H), lysine (K), arginine (R), glutamine (Q), asparagine (N), glutamic acid (E), aspartic acid (D), serine(S), and threonine (T).

The terms “hydrophilic” and “hydrophobic” refer to the degree of affinity that a substance has with water. A hydrophilic substance has a strong affinity for water, tending to dissolve in, mix with, or be wetted by water, while a hydrophobic substance substantially lacks affinity for water, tending to repel and not absorb water and tending not to dissolve in or mix with or be wetted by water. Amino acids can be characterized based on their hydrophobicity. Examples of “hydrophilic amino acids” are arginine, lysine, threonine, alanine, asparagine, and glutamine. Of particular interest are the hydrophilic amino acids aspartate, glutamate, and serine, and glycine. Examples of “hydrophobic amino acids” are tryptophan, tyrosine, phenylalanine, methionine, leucine, isoleucine, and valine.

A “host cell” includes an individual cell or cell culture which can be or has been a recipient for the subject vectors. Host cells include progeny of a single host cell. The progeny may not necessarily be completely identical (in morphology or in genomic of total DNA complement) to the original parent cell due to natural.

A “chimeric” protein contains at least one fusion polypeptide comprising regions in a different position in the sequence than that which occurs in nature. The regions may normally exist in separate proteins and are brought together in the fusion polypeptide; or they may normally exist in the same protein but are placed in a new arrangement in the fusion polypeptide. A chimeric protein may be created, for example, by chemical synthesis, or by creating and translating a polynucleotide in which the peptide regions are encoded in the desired relationship.

“Conjugated”, “linked,” “fused,” and “fusion” are used interchangeably herein. These terms refer to the joining together of two more chemical elements or components, by whatever means including chemical conjugation or recombinant means.

The terms “polynucleotides”, “nucleic acids”, “nucleotides” and “oligonucleotides” are used interchangeably. They refer to a polymeric form of nucleotides of any length, either deoxyribonucleotides or ribonucleotides, or analogs thereof. Polynucleotides may have any three-dimensional structure, and may perform any function, known or unknown. The following are non-limiting examples of polynucleotides; coding or non-coding regions of a gene or gene fragment, loci (locus) defined from linkage analysis, exons, introns, messenger RNA (mRNA), transfer RNA, ribosomal RNA, ribozymes, cDNA, recombinant polynucleotides, branched polynucleotides, plasmids, vectors, isolated DNA of any sequence, isolated RNA of any sequence, nucleic acid probes, and primers. A polynucleotide may comprise modified nucleotides, such as methylated nucleotides and nucleotide analogs. If present, modifications to the nucleotide structure may be imparted before or after assembly of the polymer. The sequence of nucleotides may be interrupted by non-nucleotide components. A polynucleotide may be further modified after polymerization, such as by conjugation with a labeling component.

The term “complement of a polynucleotide” denotes a polynucleotide molecule having a complementary base sequence and reverse orientation as compared to a reference sequence, such that it could hybridize with a reference sequence with complete fidelity.

“Recombinant” as applied to a polynucleotide means that the polynucleotide is the product of various combinations of in vitro cloning, restriction and/or ligation steps, and other procedures that result in a construct that can potentially be expressed in a host cell.

“Homology” or “homologous” refers to sequence similarity or interchangeability between two or more polynucleotide sequences or two or more polypeptide sequences. When using a program such as BestFit to determine sequence identity, similarity or homology between two different amino acid sequences, the default settings may be used, or an appropriate scoring matrix, such as blosum45 or blosum80, may be selected to optimize identity, similarity or homology scores. Preferably, polynucleotides that are homologous are those which hybridize under stringent conditions as defined herein and have at least 70%, preferably at least 80%, more preferably at least 90%, more preferably 95%, more preferably 97%, more preferably 98%, and even more preferably 99% sequence identity to those sequences.

The terms “percent identity” and “% identity,” as applied to polynucleotide sequences, refer to the percentage of residue matches between at least two polynucleotide sequences aligned using a standardized algorithm. Such an algorithm may insert, in a standardized and reproducible way, gaps in the sequences being compared in order to optimize alignment between two sequences, and therefore achieve a more meaningful comparison of the two sequences. Percent identity may be measured over the length of an entire defined polynucleotide sequence, for example, as defined by a particular SEQ ID number, or may be measured over a shorter length, for example, over the length of a fragment taken from a larger, defined polynucleotide sequence, for instance, a fragment of at least 45, at least 60, at least 90, at least 120, at least 150, at least 210 or at least 450 contiguous residues. Such lengths are exemplary only, and it is understood that any fragment length supported by the sequences shown herein, in the tables, figures or Sequence Listing, may be used to describe a length over which percentage identity may be measured.

“Percent (%) amino acid sequence identity,” with respect to the polypeptide sequences identified herein, is defined as the percentage of amino acid residues in a query sequence that are identical with the amino acid residues of a second, reference polypeptide sequence or a portion thereof, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared. Percent identity may be measured over the length of an entire defined polypeptide sequence, for example, as defined by a particular SEQ ID number, or may be measured over a shorter length, for example, over the length of a fragment taken from a larger, defined polypeptide sequence, for instance, a fragment of at least 15, at least 20, at least 30, at least 40, at least 50, at least 70 or at least 150 contiguous residues. Such lengths are exemplary only, and it is understood that any fragment length supported by the sequences shown herein, in the tables, figures or Sequence Listing, may be used to describe a length over which percentage identity may be measured.

A “vector” is a nucleic acid molecule, preferably self-replicating in an appropriate host, which transfers an inserted nucleic acid molecule into and/or between host cells. The term includes vectors that function primarily for insertion of DNA or RNA into a cell, replication of vectors that function primarily for the replication of DNA or RNA, and expression vectors that function for transcription and/or translation of the DNA or RNA. Also included are vectors that provide more than one of the above functions. An “expression vector” is a polynucleotide which, when introduced into an appropriate host cell, can be transcribed and translated into a polypeptide(s). An “expression system” usually connotes a suitable host cell comprised of an expression vector that can function to yield a desired expression product.

The term “t_{1/2}” as used herein means the terminal half-life calculated as ln(2)/Kₑₗ, Kₑₗ is the terminal elimination rate constant calculated by linear regression of the terminal linear portion of the log concentration vs. time curve. Half-life typically refers to the time required for half the quantity of an administered substance deposited in a living organism to be metabolized or eliminated by normal biological processes. The terms “t_{1/2}”, “terminal half-life”, “elimination half-life” and “circulating half-life” are used interchangeably herein.

“Physiological conditions” refer to a set of conditions in a living host as well as in vitro conditions, including temperature, salt concentration, pH, that mimic those conditions of a living subject. A host of physiologically relevant conditions for use in in vitro assays have been established. Generally, a physiological buffer contains a physiological concentration of salt and is adjusted to a neutral pH ranging from about 6.5 to about 7.8, and preferably from about 7.0 to about 7.5. A variety of physiological buffers is listed in Sambrook et al. (1989). Physiologically relevant temperature ranges from about 25° C. to about 38° C., and preferably from about 35° C. to about 37° C.

The term “antagonist,” as used herein, includes any molecule that partially or fully blocks, inhibits, or neutralizes a biological activity of a native polypeptide disclosed herein. Methods for identifying antagonists of a polypeptide may comprise contacting a native polypeptide with a candidate antagonist molecule and measuring a detectable change in one or more biological activities normally associated with the native polypeptide. In the context of the present invention, antagonists may include proteins, nucleic acids, carbohydrates, antibodies or any other molecules that decrease the effect of a biologically active protein.

The term “agonist” is used in the broadest sense and includes any molecule that mimics a biological activity of a native polypeptide disclosed herein. Suitable agonist molecules specifically include agonist antibodies or antibody fragments, fragments or amino acid sequence variants of native polypeptides, peptides, small organic molecules, etc. Methods for identifying agonists of a native polypeptide may comprise contacting a native polypeptide with a candidate agonist molecule and measuring a detectable change in one or more biological activities normally associated with the native polypeptide.

“Activity” for the purposes herein refers to an action or effect of a component of a fusion protein consistent with that of the corresponding native biologically active protein, wherein “biological activity” refers to an in vitro or in vivo biological function or effect, including but not limited to receptor binding, antagonist activity, agonist activity, or a cellular or physiologic response.

As used herein, “treatment” or “treating,” “palliating,” and “ameliorating” are used interchangeably herein. These terms refer to an approach for obtaining beneficial or desired results including but not limited to a therapeutic benefit and/or a prophylactic benefit. By “therapeutic benefit” is meant eradication or amelioration of the underlying disorder being treated. Also, a therapeutic benefit is achieved with the eradication or amelioration of one or more of the physiological symptoms associated with the underlying disease condition such that an improvement is observed in the subject, notwithstanding that the subject may still be afflicted with the underlying disorder. For prophylactic benefit, the compositions may be administered to a subject at risk of developing a particular disease condition, or to a subject reporting one or more of the physiological symptoms of a disease, even though a diagnosis of this disease may not have been made.

A “therapeutic effect,” as used herein, refers to a physiologic effect, including but not limited to the cure, mitigation, amelioration, or prevention of disease condition in humans or other animals, or to otherwise enhance physical or mental wellbeing of humans or animals, caused by a fusion polypeptide of the invention other than the ability to induce the production of an antibody against an antigenic epitope possessed by the biologically active protein. Determination of a therapeutically effective amount is well within the capability of those skilled in the art, especially in light of the detailed disclosure provided herein.

The terms “therapeutically effective amount” and “therapeutically effective dose,” as used herein, refers to an amount of a biologically active protein, either alone or as a part of a fusion protein composition, that is capable of having any detectable, beneficial effect on any symptom, aspect, measured parameter or characteristics of a disease state or condition when administered in one or repeated doses to a subject. Such effect need not be absolute to be beneficial. The disease condition can refer to a disorder or a disease.

The term “therapeutically effective dose regimen,” as used herein, refers to a schedule for consecutively administered doses of a biologically active protein, either alone or as a part of a fusion protein composition, wherein the doses are given in therapeutically effective amounts to result in sustained beneficial effect on any symptom, aspect, measured parameter or characteristics of a disease state or condition.

The term “subject,” “individual” or “patient” as used herein refers to any animals that can be used in the present disclosure, including but not limited to human, primate, rodent, canine, feline, equine, ovine, porcine, and the like.

The term “in vivo” as used herein refers to an event that takes place in a subject's body.

The term “in vitro” as used herein refers to an event that takes places outside of a subject's body. In some embodiments, an in vitro assay encompasses any assay run outside of a subject assay. In vitro assays encompass cell-based assays in which cells alive or dead are employed. In vitro assays also encompass a cell-free assay in which no intact cells are employed,

### Polypeptide

In one aspect, provided is an artificial polypeptide of formula X—Y (I).

In some embodiments, X of formula (I) can be a hydrophilic moiety, at least 50% of which are selected from R, K, N, D, Q, E, and H. In some embodiments, X of formula (I) can be a hydrophilic moiety, at least 55% of which are selected from R, K, N, D, Q, E, and H. In some embodiments, X of formula (I) can be a hydrophilic moiety, at least 60% of which are selected from R, K, N, D, Q, E, and H. In some embodiments, X of formula (I) can be a hydrophilic moiety, at least 65% of which are selected from R, K, N, D, Q, E, and H. In some embodiments, X of formula (I) can be a hydrophilic moiety, at least 70% of which are selected from R, K, N, D, Q, E, and H. In some embodiments, X of formula (I) can be a hydrophilic moiety, at least 75% of which are selected from R, K, N, D, Q, E, and H. In some embodiments, X of formula (I) can be a hydrophilic moiety, at least 80% of which are selected from R, K, N, D, Q, E, and H. In some embodiments, X of formula (I) can be a hydrophilic moiety, at least 85% of which are selected from R, K, N, D, Q, E, and H. In some embodiments, X of formula (I) can be a hydrophilic moiety, at least 90% of which are selected from R, K, N, D, Q, E, and H.

In some embodiments, X of formula (I) can be a moiety comprising a sequence having at least 80% identity with SEQ ID NO. 1. In some embodiments, X is a moiety comprising a sequence having at least 85% identity with SEQ ID NO. 1. In some embodiments, X is a moiety comprising a sequence having at least 90% identity with SEQ ID NO. 1. In some embodiments, X is a moiety comprising a sequence having at least 95% identity with SEQ ID NO. 1. In some embodiments, X is a moiety comprising a sequence having at least 96%, at least 97%, at least 98%, or at least 99% identity with SEQ ID NO. 1. In some embodiments, X is a moiety comprising the sequence of SEQ ID NO. 1. In some embodiments, X is a moiety that is the sequence of SEQ ID NO. 1.

In some embodiments, Y of formula (I) can be a hydrophobic moiety, at least 50% of which are selected from I, V, L, F, C, M, and A. In some embodiments, Y of formula (I) can be a hydrophobic moiety, at least 55% of which are selected from I, V, L, F, C, M, and A. In some embodiments, Y of formula (I) can be a hydrophobic moiety, at least 60% of which are selected from I, V, L, F, C, M, and A. In some embodiments, Y of formula (I) can be a hydrophobic moiety, at least 65% of which are selected from I, V, L, F, C, M, and A. In some embodiments, Y of formula (I) can be a hydrophobic moiety, at least 70% of which are selected from I, V, L, F, C, M, and A. In some embodiments, Y of formula (I) can be a hydrophobic moiety, at least 75% of which are selected from I, V, L, F, C, M, and A. In some embodiments, Y of formula (I) can be a hydrophobic moiety, at least 80% of which are selected from I, V, L, F, C, M, and A. In some embodiments, Y of formula (I) can be a hydrophobic moiety, at least 85% of which are selected from I, V, L, F, C, M, and A. In some embodiments, Y of formula (I) can be a hydrophobic moiety, at least 90% of which are selected from I, V, L, F, C, M, and A.

In some embodiments, Y of formula (I) can be a moiety comprising 10 to 50 amino acids, and a total number of hydrophobic amino acids of Y is 5. In some embodiments, Y of formula (I) can be a moiety comprising 10 to 50 amino acids, and a total number of hydrophobic amino acids of Y is more than 5. In some embodiments, Y of formula (I) can be a moiety comprising 10 to 50 amino acids, and a total number of hydrophobic amino acids of Y is 6. In some embodiments, Y of formula (I) can be a moiety comprising 10 to 50 amino acids, and a total number of hydrophobic amino acids of Y is more than 6. In some embodiments, Y of formula (I) can be a moiety comprising 10 to 50 amino acids, and a total number of hydrophobic amino acids of Y is 7. In some embodiments, Y of formula (I) can be a moiety comprising 10 to 50 amino acids, and a total number of hydrophobic amino acids of Y is more than 7. In some embodiments, Y of formula (I) can be a moiety comprising 10 to 50 amino acids, and a total number of hydrophobic amino acids of Y is 8. In some embodiments, Y of formula (I) can be a moiety comprising 10 to 50 amino acids, and a total number of hydrophobic amino acids of Y is more than 8. In some embodiments, Y of formula (I) can be a moiety comprising 10 to 50 amino acids, and a total number of hydrophobic amino acids of Y is 9. In some embodiments, Y of formula (I) can be a moiety comprising 10 to 50 amino acids, and a total number of hydrophobic amino acids of Y is more than 9. In some embodiments, Y of formula (I) can be a moiety comprising 10 to 50 amino acids, and a total number of hydrophobic amino acids of Y is 10. In some embodiments, Y of formula (I) can be a moiety comprising 10 to 50 amino acids, and a total number of hydrophobic amino acids of Y is more than 10. In some embodiments, Y of formula (I) can be a moiety comprising 10 to 50 amino acids, and a total number of hydrophobic amino acids of Y is 11. In some embodiments, Y of formula (I) can be a moiety comprising 10 to 50 amino acids, and a total number of hydrophobic amino acids of Y is more than 11. In some embodiments, Y of formula (I) can be a moiety comprising 10 to 50 amino acids, and a total number of hydrophobic amino acids of Y is 12. In some embodiments, Y of formula (I) can be a moiety comprising 10 to 50 amino acids, and a total number of hydrophobic amino acids of Y is more than 12. In some embodiments, Y of formula (I) can be a moiety comprising 10 to 50 amino acids, and a total number of hydrophobic amino acids of Y is 13. In some embodiments, Y of formula (I) can be a moiety comprising 10 to 50 amino acids, and a total number of hydrophobic amino acids of Y is more than 13. In some embodiments, Y of formula (I) can be a moiety comprising 10 to 50 amino acids, and a total number of hydrophobic amino acids of Y is 14. In some embodiments, Y of formula (I) can be a moiety comprising 10 to 50 amino acids, and a total number of hydrophobic amino acids of Y is more than 14. In some embodiments, Y of formula (I) can be a moiety comprising 10 to 50 amino acids, and a total number of hydrophobic amino acids of Y is 15. In some embodiments, Y of formula (I) can be a moiety comprising 10 to 50 amino acids, and a total number of hydrophobic amino acids of Y is more than 15.

In some embodiments, Y of formula (I) can be a moiety comprising 10 to 50 amino acids, and a total number of I, V, L, F, C, M, and A of Y is 5. In some embodiments, Y of formula (I) can be a moiety comprising 10 to 50 amino acids, and a total number of I, V, L, F, C, M, and A of Y is more than 5. In some embodiments, Y of formula (I) can be a moiety comprising 10 to 50 amino acids, and a total number of I, V, L, F, C, M, and A of Y is 6. In some embodiments, Y of formula (I) can be a moiety comprising 10 to 50 amino acids, and a total number of I, V, L, F, C, M, and A of Y is more than 6. In some embodiments, Y of formula (I) can be a moiety comprising 10 to 50 amino acids, and a total number of I, V, L, F, C, M, and A of Y is 7. In some embodiments, Y of formula (I) can be a moiety comprising 10 to 50 amino acids, and a total number of I, V, L, F, C, M, and A of Y is more than 7. In some embodiments, Y of formula (I) can be a moiety comprising 10 to 50 amino acids, and a total number of I, V, L, F, C, M, and A of Y is 8. In some embodiments, Y of formula (I) can be a moiety comprising 10 to 50 amino acids, and a total number of I, V, L, F, C, M, and A of Y is more than 8. In some embodiments, Y of formula (I) can be a moiety comprising 10 to 50 amino acids, and a total number of I, V, L, F, C, M, and A of Y is 9. In some embodiments, Y of formula (I) can be a moiety comprising 10 to 50 amino acids, and a total number of I, V, L, F, C, M, and A of Y is more than 9. In some embodiments, Y of formula (I) can be a moiety comprising 10 to 50 amino acids, and a total number of I, V, L, F, C, M, and A of Y is 10. In some embodiments, Y of formula (I) can be a moiety comprising 10 to 50 amino acids, and a total number of I, V, L, F, C, M, and A of Y is more than 10. In some embodiments, Y of formula (I) can be a moiety comprising 10 to 50 amino acids, and a total number of I, V, L, F, C, M, and A of Y is 11. In some embodiments, Y of formula (I) can be a moiety comprising 10 to 50 amino acids, and a total number of I, V, L, F, C, M, and A of Y is more than 11. In some embodiments, Y of formula (I) can be a moiety comprising 10 to 50 amino acids, and a total number of I, V, L, F, C, M, and A of Y is 12. In some embodiments, Y of formula (I) can be a moiety comprising 10 to 50 amino acids, and a total number of I, V, L, F, C, M, and A of Y is more than 12. In some embodiments, Y of formula (I) can be a moiety comprising 10 to 50 amino acids, and a total number of I, V, L, F, C, M, and A of Y is 13. In some embodiments, Y of formula (I) can be a moiety comprising 10 to 50 amino acids, and a total number of I, V, L, F, C, M, and A of Y is more than 13. In some embodiments, Y of formula (I) can be a moiety comprising 10 to 50 amino acids, and a total number of I, V, L, F, C, M, and A of Y is 14. In some embodiments, Y of formula (I) can be a moiety comprising 10 to 50 amino acids, and a total number of I, V, L, F, C, M, and A of Y is more than 14. In some embodiments, Y of formula (I) can be a moiety comprising 10 to 50 amino acids, and a total number of I, V, L, F, C, M, and A of Y is 15. In some embodiments, Y of formula (I) can be a moiety comprising 10 to 50 amino acids, and a total number of I, V, L, F, C, M, and A of Y is more than 15.

In some embodiments, Y of formula (I) can be a moiety comprising 10 to 50 amino acids, and a total number of Cysteine (C) of Y is 5. In some embodiments, Y of formula (I) can be a moiety comprising 10 to 50 amino acids, and a total number of Cysteine (C) of Y is less than 5. In some embodiments, Y of formula (I) can be a moiety comprising 10 to 50 amino acids, and a total number of Cysteine (C) of Y is 4. In some embodiments, Y of formula (I) can be a moiety comprising 10 to 50 amino acids, and a total number of Cysteine (C) of Y is less than 4. In some embodiments, Y of formula (I) can be a moiety comprising 10 to 50 amino acids, and a total number of Cysteine (C) of Y is 3. In some embodiments, Y of formula (I) can be a moiety comprising 10 to 50 amino acids, and a total number of Cysteine (C) of Y is less than 3. In some embodiments, Y of formula (I) can be a moiety comprising 10 to 50 amino acids, and a total number of Cysteine (C) of Y is 2. In some embodiments, Y of formula (I) can be a moiety comprising 10 to 50 amino acids, and a total number of Cysteine (C) of Y is less than 2. In some embodiments, Y of formula (I) can be a moiety comprising 10 to 50 amino acids, and a total number of Cysteine (C) of Y is 1. In some embodiments, Y of formula (I) can be a moiety comprising 10 to 50 amino acids, and a total number of Cysteine (C) of Y is less than 1. In some embodiments, Y of formula (I) can be a moiety comprising 10 to 50 amino acids, and a total number of Cysteine (C) of Y is 0.

In some embodiments, a total number of hydrophilic amino acids in Y of formula (I) is 5. In some embodiments, a total number of hydrophilic amino acids in Y of formula (I) is no more than 5. In some embodiments, a total number of hydrophilic amino acids in Y of formula (I) is 4. In some embodiments, a total number of hydrophilic amino acids in Y of formula (I) is no more than 4. In some embodiments, a total number of hydrophilic amino acids in Y of formula (I) is 3. In some embodiments, a total number of hydrophilic amino acids in Y of formula (I) is no more than 3. In some embodiments, a total number of hydrophilic amino acids in Y of formula (I) is 2. In some embodiments, a total number of hydrophilic amino acids in Y of formula (I) is no more than 2. In some embodiments, a total number of hydrophilic amino acids in Y of formula (I) is 1. In some embodiments, a total number of hydrophilic amino acids in Y of formula (I) is no more than 1.

In some embodiments, a total number of R, K, N, D, Q, E, and H in Y of formula (I) is 5. In some embodiments, a total number of R, K, N, D, Q, E, and H in Y of formula (I) is no more than 5. In some embodiments, a total number of hydrophilic amino acids in Y of formula (I) is 4. In some embodiments, a total number of R, K, N, D, Q, E, and H in Y of formula (I) is no more than 4. In some embodiments, a total number of R, K, N, D, Q, E, and H in Y of formula (I) is 3. In some embodiments, a total number of R, K, N, D, Q, E, and H in Y of formula (I) is no more than 3. In some embodiments, a total number of R, K, N, D, Q, E, and H in Y of formula (I) is 2. In some embodiments, a total number of R, K, N, D, Q, E, and H in Y of formula (I) is no more than 2. In some embodiments, a total number of R, K, N, D, Q, E, and H in Y of formula (I) is 1. In some embodiments, a total number of R, K, N, D, Q, E, and H in Y of formula (I) is no more than 1.

In some embodiments of the artificial polypeptide, X of formula (I) comprises a sequence having at least 70% identity with any one selected from SEQ ID NOs. 81-83. In some embodiments of the artificial polypeptide, X of formula (I) comprises a sequence having at least 75% identity with any one selected from SEQ ID NOs. 81-83. In some embodiments of the artificial polypeptide, X of formula (I) comprises a sequence having at least 80% identity with any one selected from SEQ ID NOs. 81-83. In some embodiments of the artificial polypeptide, X of formula (I) comprises a sequence having at least 85% identity with any one selected from SEQ ID NOs. 81-83. In some embodiments of the artificial polypeptide, X of formula (I) comprises a sequence having at least 90% identity with any one selected from SEQ ID NOs. 81-83. In some embodiments of the artificial polypeptide, X of formula (I) comprises a sequence having at least 95% identity with any one selected from SEQ ID NOs. 81-83. In some embodiments of the artificial polypeptide, X of formula (I) comprises a sequence having at least 96%, at least 97%, at least 98%, or at least 99% identity with any one selected from SEQ ID NOs. 81-83. In some embodiments of the artificial polypeptide, X of formula (I) comprises a sequence of any one selected from SEQ ID NOs. 81-83. In some embodiments of the artificial polypeptide, X of formula (I) is a sequence of any one selected from SEQ ID NOs. 81-83.

In some embodiments of the artificial polypeptide, Y of formula (I) comprises a sequence having at least 70% identity with any one selected from SEQ ID NOs. 3-15, 58-61 and 84-85. In some embodiments of the artificial polypeptide, Y of formula (I) comprises a sequence having at least 75% identity with any one selected from SEQ ID NOs. 3-15, 58-61 and 84-85. In some embodiments of the artificial polypeptide, Y of formula (I) comprises a sequence having at least 80% identity with any one selected from SEQ ID NOs. 3-15, 58-61 and 84-85. In some embodiments of the artificial polypeptide, Y of formula (I) comprises a sequence having at least 85% identity with any one selected from SEQ ID NOs. 3-15, 58-61 and 84-85. In some embodiments of the artificial polypeptide, Y of formula (I) comprises a sequence having at least 90% identity with any one selected from SEQ ID NOs. 3-15, 58-61 and 84-85. In some embodiments of the artificial polypeptide, Y of formula (I) comprises a sequence having at least 95% identity with any one selected from SEQ ID NOs. 3-15, 58-61 and 84-85. In some embodiments of the artificial polypeptide, Y of formula (I) comprises a sequence having at least 96%, at least 97%, at least 98%, or at least 99% identity with any one selected from SEQ ID NOs. 3-15, 58-61 and 84-85. In some embodiments of the artificial polypeptide, Y of formula (I) comprises a sequence of any one selected from SEQ ID NOs. 3-15, 58-61 and 84-85. In some embodiments of the artificial polypeptide, Y of formula (I) is a sequence of any one selected from SEQ ID NOs, 3-15, 58-61 and 84-85.

In some embodiments of the artificial polypeptide of formula (I), the polypeptide comprises a sequence having at least 70% identity with any one selected from SEQ ID NOs. 29-41, 54-57 and 91-95. In some embodiments of the artificial polypeptide of formula (I), the polypeptide comprises a sequence having at least 75% identity with any one selected from SEQ ID NOs. 29-41, 54-57 and 91-95. In some embodiments of the artificial polypeptide of formula (I), the polypeptide comprises a sequence having at least 80% identity with any one selected from SEQ ID NOs. 29-41, 54-57 and 91-95. In some embodiments of the artificial polypeptide of formula (I), the polypeptide comprises a sequence having at least 85% identity with any one selected from SEQ ID NOs. 29-41, 54-57 and 91-95. In some embodiments of the artificial polypeptide of formula (I), the polypeptide comprises a sequence having at least 90% identity with any one selected from SEQ ID NOs. 29-41, 54-57 and 91-95. In some embodiments of the artificial polypeptide of formula (I), the polypeptide comprises a sequence having at least 95% identity with any one selected from SEQ ID NOs. 29-41, 54-57 and 91-95. In some embodiments of the artificial polypeptide of formula (I), the polypeptide comprises a sequence having at least 96%, at least 97%, at least 98%, or at least 99% identity with any one selected from SEQ ID NOs. 29-41, 54-57 and 91-95. In some embodiments of the artificial polypeptide of formula (I), the polypeptide comprises a sequence of any one of SEQ ID NOs. 29-41, 54-57 and 91-95. In some embodiments of the artificial polypeptide of formula (I), the polypeptide is a sequence of any one of SEQ ID NOs. 29-41, 54-57 and 91-95.

In another aspect, provided is an artificial polypeptide of formula X—Y (II).

In some embodiments, X of formula (II) can be a hydrophilic moiety, at least 50% of which are selected from R, K, N, D, Q, E, and H. In some embodiments, X of formula (II) can be a hydrophilic moiety, at least 55% of which are selected from R, K, N, D, Q, E, and H. In some embodiments, X of formula (I) can be a hydrophilic moiety, at least 60% of which are selected from R, K, N, D, Q, E, and H. In some embodiments, X of formula (II) can be a hydrophilic moiety, at least 65% of which are selected from R, K, N, D, Q, E, and H. In some embodiments, X of formula (II) can be a hydrophilic moiety, at least 70% of which are selected from R, K, N, D, Q, E, and H. In some embodiments, X of (II) can be a hydrophilic moiety, at least 75% of which are selected from R, K, N, D, Q, E, and H. In some embodiments, X of formula (II) can be a hydrophilic moiety, at least 80% of which are selected from R, K, N, D, Q, E, and H. In some embodiments, X of formula (II) can be a hydrophilic moiety, at least 85% of which are selected from R, K, N, D, Q, E, and H. In some embodiments, X of formula (II) can be a hydrophilic moiety, at least 90% of which are selected from R, K, N, D, Q, E, and H.

In some embodiments, X of formula (II) can be a hydrophilic moiety comprising one or more Arginine (R). For example, X of formula (II) can be a hydrophilic moiety comprising at least one, at least two, at least three, at least four, at least five, at least six, at least seven, at least eight, at least nine, or at least ten Arginine (R). In some embodiments, X of formula (II) can be a hydrophilic moiety comprising one or more Lysine (K). For example, X of formula (II) can be a hydrophilic moiety comprising at least one, at least two, at least three, at least four, at least five, at least six, at least seven, at least eight, at least nine, or at least ten Lysine (K). In some embodiments, X of formula (II) can be a hydrophilic moiety comprising one or more Asparagine (N). For example, X of formula (II) can be a hydrophilic moiety comprising at least one, at least two, at least three, at least four, at least five, at least six, at least seven, at least eight, at least nine, or at least ten Asparagine (N). In some embodiments, X of formula (II) can be a hydrophilic moiety comprising one or more Aspartic Acid (D). For example, X of formula (II) can be a hydrophilic moiety comprising at least one, at least two, at least three, at least four, at least five, at least six, at least seven, at least eight, at least nine, or at least ten Aspartic Acid (D). In some embodiments, X of formula (II) can be a hydrophilic moiety comprising one or more Glutamine (Q). For example, X of formula (II) can be a hydrophilic moiety comprising at least one, at least two, at least three, at least four, at least five, at least six, at least seven, at least eight, at least nine, or at least ten Glutamine (Q). In some embodiments, X of formula (II) can be a hydrophilic moiety comprising one or more Glutamic Acid (E). For example, X of formula (II) can be a hydrophilic moiety comprising at least one, at least two, at least three, at least four, at least five, at least six, at least seven, at least eight, at least nine, or at least ten Glutamic Acid (E). In some embodiments, X of formula (II) can be a hydrophilic moiety comprising one or more Histidine (H). For example, X of formula (II) can be a hydrophilic moiety comprising at least one, at least two, at least three, at least four, at least five, at least six, at least seven, at least eight, at least nine, or at least ten Histidine (H).

In some embodiments, X of formula (II) can be a moiety comprising a sequence having at least 80% identity with SEQ ID NO. 1. In some embodiments, X is a moiety comprising a sequence having at least 85% identity with SEQ ID NO. 1. In some embodiments, X is a moiety comprising a sequence having at least 90% identity with SEQ ID NO. 1. In some embodiments, X is a moiety comprising a sequence having at least 95% identity with SEQ ID NO. 1. In some embodiments, X is a moiety comprising a sequence having at least 96%, at least 97%, at least 98%, or at least 99% identity with SEQ ID NO. 1. In some embodiments, X is a moiety comprising the sequence of SEQ ID NO. 1. In some embodiments, X is a moiety that is the sequence of SEQ ID NO. 1.

In some embodiments of the artificial polypeptide of formula (II), Y comprises a sequence having at most 50% identity with SEQ ID NO. 2. In some embodiments, Y comprises a sequence having at most 55% identity with SEQ ID NO. 2. In some embodiments, Y comprises a sequence having at most 60% identity with SEQ ID NO. 2. In some embodiments, Y comprises a sequence having at most 65% identity with SEQ ID NO, 2. In some embodiments, Y comprises a sequence having at most 70% identity with SEQ ID NO. 2. In some embodiments, Y comprises a sequence having at most 75% identity with SEQ ID NO. 2. In some embodiments, Y comprises a sequence having at most 80% identity with SEQ ID NO. 2. In some embodiments, Y comprises a sequence having at most 85% identity with SEQ ID NO. 2. In some embodiments, Y comprises a sequence having at most 90% identity with SEQ ID NO. 2.

In some embodiments of the artificial polypeptide of formula (II), Y comprises a sequence having at least 70% identity with any one selected from SEQ ID NOs. 16-18. In some embodiments of the artificial polypeptide, Y comprises a sequence having at least 75% identity with any one selected from SEQ ID NOs. 16-18. In some embodiments of the artificial polypeptide, Y comprises a sequence having at least 80% identity with any one selected from SEQ ID NOs. 16-18. In some embodiments of the artificial polypeptide, Y comprises a sequence having at least 85% identity with any one selected from SEQ ID NOs. 16-18. In some embodiments of the artificial polypeptide, Y comprises a sequence having at least 90% identity with any one selected from SEQ ID NOs. 16-18. In some embodiments of the artificial polypeptide, Y comprises a sequence having at least 95% identity with any one selected from SEQ ID NOs. 16-18. In some embodiments of the artificial polypeptide, Y comprises a sequence having at least 96%, at least 97%, at least 98%, or at least 99% identity with any one selected from SEQ ID NOs. 16-18. In some embodiments of the artificial polypeptide, Y comprises a sequence of any one selected from SEQ ID NOs. 16-18. In some embodiments of the artificial polypeptide, Y is a sequence of any one selected from SEQ ID NOs. 16-18.

In some embodiments of the artificial polypeptide of formula (II), the artificial polypeptide comprises a sequence having at least 70% identity with any one selected from SEQ ID NOs. 42-44. In some embodiments of the artificial polypeptide, the artificial polypeptide comprises a sequence having at least 75% identity with any one selected from SEQ ID NOs. 42-44. In some embodiments of the artificial polypeptide, the artificial polypeptide comprises a sequence having at least 80% identity with any one selected from SEQ ID NOs. 42-44. In some embodiments of the artificial polypeptide, the artificial polypeptide comprises a sequence having at least 85% identity with any one selected from SEQ ID NOs. 42-44. In some embodiments of the artificial polypeptide, the artificial polypeptide comprises a sequence having at least 90% identity with any one selected from SEQ ID NOs. 42-44. In some embodiments of the artificial polypeptide, the artificial polypeptide comprises a sequence having at least 95% identity with any one selected from SEQ ID NOs. 42-44. In some embodiments of the artificial polypeptide, the artificial polypeptide comprises a sequence having at least 96%, at least 97%, at least 98%, or at least 99% identity with any one selected from SEQ ID NOs. 42-44. In some embodiments of the artificial polypeptide, the artificial polypeptide comprises a sequence of any one selected from SEQ ID NOs. 42-44. In some embodiments of the artificial polypeptide, the artificial polypeptide is a sequence of any one selected from SEQ ID NOs. 42-44.

In another aspect, provided is an artificial polypeptide of formula X—Y (III).

In some embodiments, X of formula (III) is hydrophilic moiety, at least 50% of which are selected from H, R, K, D, Q, N and E. In some embodiments, X of formula (III) can be a hydrophilic moiety, at least 55% of which are selected from R, K, N, D, Q, E, and H. In some embodiments, X of formula (III) can be a hydrophilic moiety, at least 60% of which are selected from R, K, N, D, Q, E, and H. In some embodiments, X of formula (III) can be a hydrophilic moiety, at least 65% of which are selected from R, K, N, D, Q, E, and H. In some embodiments, X of formula (III) can be a hydrophilic moiety, at least 70% of which are selected from R, K, N, D, Q, E, and H. In some embodiments, X of formula (III) can be a hydrophilic moiety, at least 75% of which are selected from R, K, N, D, Q, E, and H. In some embodiments, X of formula (III) can be a hydrophilic moiety, at least 80% of which are selected from R, K, N, D, Q, E, and H. In some embodiments, X of formula (III) can be a hydrophilic moiety, at least 85% of which are selected from R, K, N, D, Q, E, and H. In some embodiments, X of formula (III) can be a hydrophilic moiety, at least 90% of which are selected from R, K, N, D, Q, E, and H.

In some embodiments, X of formula (III) is a moiety comprising 40 to 65 amino acids, and a total number of H, R, K, D, Q, N and E in X is less than 33. In some embodiments, X of formula (III) is a moiety comprising 40 to 65 amino acids, and a total number of H, R, K, D, Q, N and E in X is less than 32. In some embodiments, X of formula (III) is a moiety comprising 40 to 65 amino acids, and a total number of H, R, K, D, Q, N and E in X is less than 31. In some embodiments, X of formula (III) is a moiety comprising 40 to 65 amino acids, and a total number of H, R, K, D, Q, N and E in X is less than 30. In some embodiments, X of formula (III) is a moiety comprising 40 to 65 amino acids, and a total number of H, R, K, D, Q, N and E in X is less than 29. In some embodiments, X of formula (III) is a moiety comprising 40 to 65 amino acids, and a total number of H, R, K, D, Q, N and E in X is less than 28. In some embodiments, X of formula (III) is a moiety comprising 40 to 65 amino acids, and a total number of H, R, K, D, Q, N and E in X is less than 27. In some embodiments, X of formula (III) is a moiety comprising 40 to 65 amino acids, and a total number of H, R, K, D, Q, N and E in X is less than 26. In some embodiments, X of formula (III) is a moiety comprising 40 to 65 amino acids, and a total number of H, R, K, D, Q, N and E in X is less than 25. In some embodiments, X of formula (III) is a moiety comprising 40 to 65 amino acids, and a total number of H, R, K, D, Q, N and E in X is less than 24. In some embodiments, X of formula (III) is a moiety comprising 40 to 65 amino acids, and a total number of H, R, K, D, Q, N and E in X is less than 23. In some embodiments, X of formula (III) is a moiety comprising 40 to 65 amino acids, and a total number of H, R, K, D, Q, N and E in X is less than 22. In some embodiments, X of formula (III) is a moiety comprising 40 to 65 amino acids, and a total number of H, R, K, D, Q, N and E in X is less than 21. In some embodiments, X of formula (III) is a moiety comprising 40 to 65 amino acids, and a total number of H, R, K, D, Q, N and E in X is less than 20.

In some embodiments, X of formula (III) is a moiety comprising 40 to 65 amino acids, and a total number of hydrophilic amino acids in X is more than 10. In some embodiments, X of formula (III) is a moiety comprising 40 to 65 amino acids, and a total number of hydrophilic amino acids in X is more than 11. In some embodiments, X of formula (III) is a moiety comprising 40 to 65 amino acids, and a total number of hydrophilic amino acids in X is more than 12. In some embodiments, X of formula (III) is a moiety comprising 40 to 65 amino acids, and a total number of hydrophilic amino acids in X is more than 13. In some embodiments, X of formula (III) is a moiety comprising 40 to 65 amino acids, and a total number of hydrophilic amino acids in X is more than 14. In some embodiments, X of formula (III) is a moiety comprising 40 to 65 amino acids, and a total number of hydrophilic amino acids in X is more than 15. In some embodiments, X of formula (III) is a moiety comprising 40 to 65 amino acids, and a total number of hydrophilic amino acids in X is more than 16. In some embodiments, X of formula (III) is a moiety comprising 40 to 65 amino acids, and a total number of hydrophilic amino acids in X is more than 17. In some embodiments, X of formula (III) is a moiety comprising 40 to 65 amino acids, and a total number of hydrophilic amino acids in X is more than 18. In some embodiments, X of formula (III) is a moiety comprising 40 to 65 amino acids, and a total number of hydrophilic amino acids in X is more than 19. In some embodiments, X of formula (III) is a moiety comprising 40 to 65 amino acids, and a total number of hydrophilic amino acids in X is more than 20. In some embodiments, X of formula (III) is a moiety comprising 40 to 65 amino acids, and a total number of hydrophilic amino acids in X is more than 21. In some embodiments, X of formula (III) is a moiety comprising 40 to 65 amino acids, and a total number of hydrophilic amino acids in X is more than 22. In some embodiments, X of formula (III) is a moiety comprising 40 to 65 amino acids, and a total number of hydrophilic amino acids in X is more than 23. In some embodiments, X of formula (III) is a moiety comprising 40 to 65 amino acids, and a total number of hydrophilic amino acids in X is more than 24. In some embodiments, X of formula (III) is a moiety comprising 40 to 65 amino acids, and a total number of hydrophilic amino acids in X is more than 25.

In some embodiments, X of formula (III) is a moiety comprising 40 to 65 amino acids, and a total number of hydrophobic amino acids in X is no more than 15. In some embodiments, X of formula (III) is a moiety comprising 40 to 65 amino acids, and a total number of hydrophobic amino acids in X is no more than 14. In some embodiments, X of formula (III) is a moiety comprising 40 to 65 amino acids, and a total number of hydrophobic amino acids in X is no more than 13. In some embodiments, X of formula (III) is a moiety comprising 40 to 65 amino acids, and a total number of hydrophobic amino acids in X is no more than 12. In some embodiments, X of formula (III) is a moiety comprising 40 to 65 amino acids, and a total number of hydrophobic amino acids in X is no more than 11. In some embodiments, X of formula (III) is a moiety comprising 40 to 65 amino acids, and a total number of hydrophobic amino acids in X is no more than 10. In some embodiments, X of formula (III) is a moiety comprising 40 to 65 amino acids, and a total number of hydrophobic amino acids in X is no more than 5.

In some embodiments, X of formula (III) comprises a sequence having at least 70% identity with any one selected from SEQ ID NOs. 23-27. In some embodiments, X of formula (III) comprises a sequence having at least 75% identity with any one selected from SEQ ID NOs. 23-27. In some embodiments, X of formula (III) comprises a sequence having at least 80% identity with any one selected from SEQ ID NOs. 23-27. In some embodiments, X of formula (III) comprises a sequence having at least 85% identity with any one selected from SEQ ID NOs. 23-27. In some embodiments, X of formula (III) comprises a sequence having at least 90% identity with any one selected from SEQ ID NOs. 23-27. In some embodiments, X of formula (III) comprises a sequence having at least 95% identity with any one selected from SEQ ID NOs. 23-27. In some embodiments, X of formula (III) comprises a sequence having at least 96%, at least 97%, at least 98%, or at least 99% identity with any one selected from SEQ ID NOs. 23-27. In some embodiments, X of formula (III) comprises a sequence of any one selected from SEQ ID NOs. 23-27. In some embodiments, X of formula (III) is a sequence of any one selected from SEQ ID NOs. 23-27.

In some embodiments, Y of formula (III) is a moiety comprising a sequence having at least 70% identity with SEQ ID NO. 2. In some embodiments, Y of formula (III) is a moiety comprising a sequence having at least 75% identity with SEQ ID NO. 2. In some embodiments, Y of formula (III) is a moiety comprising a sequence having at least 80% identity with SEQ ID NO. 2. In some embodiments, Y of formula (III) is a moiety comprising a sequence having at least 85% identity with SEQ ID NO. 2. In some embodiments, Y of formula (III) is a moiety comprising a sequence having at least 90% identity with SEQ ID NO. 2. In some embodiments, Y of formula (III) is a moiety comprising a sequence having at least 95% identity with SEQ ID NO, 2. In some embodiments, Y of formula (III) is a moiety comprising a sequence having at least 96%, at least 97%, at least 98%, or at least 99% identity with SEQ ID NO. 2. In some embodiments, Y of formula (III) is a moiety comprising a sequence of SEQ ID NO. 2. In some embodiments, Y of formula (III) is a moiety that is a sequence of SEQ ID NO. 2.

In some embodiments, Y of formula (III) is hydrophobic moiety, at least 50% amino acids of Y are selected from I, V, L, F, C, M, and A. In some embodiments, Y of formula (III) is hydrophobic moiety, at least 55% amino acids of Y are selected from I, V, L, F, C, M, and A. In some embodiments, Y of formula (III) is hydrophobic moiety, at least 60% amino acids of Y are selected from I, V, L, F, C, M, and A. In some embodiments, Y of formula (III) is hydrophobic moiety, at least 65% amino acids of Y are selected from I, V, L, F, C, M, and A. In some embodiments, Y of formula (III) is hydrophobic moiety, at least 70% amino acids of Y are selected from I, V, L, F, C, M, and A. In some embodiments, Y of formula (III) is hydrophobic moiety, at least 75% amino acids of Y are selected from I, V, L, F, C, M, and A. In some embodiments, Y of formula (III) is hydrophobic moiety, at least 80% amino acids of Y are selected from I, V, L, F, C, M, and A. In some embodiments, Y of formula (III) is hydrophobic moiety, at least 85% amino acids of Y are selected from I, V, L, F, C, M, and A. In some embodiments, Y of formula (III) is hydrophobic moiety, at least 90% amino acids of Y are selected from I, V, L, F, C, M, and A.

In some embodiments, the artificial polypeptide of formula (III) comprises a sequence having at least 70% identity with any one selected from SEQ ID NOs. 49-53. In some embodiments, the artificial polypeptide of formula (III) comprises a sequence having at least 75% identity with any one selected from SEQ ID NOs. 49-53. In some embodiments, the artificial polypeptide of formula (III) comprises a sequence having at least 80% identity with any one selected from SEQ ID NOs. 49-53. In some embodiments, the artificial polypeptide of formula (III) comprises a sequence having at least 85% identity with any one selected from SEQ ID NOs. 49-53. In some embodiments, the artificial polypeptide of formula (III) comprises a sequence having at least 90% identity with any one selected from SEQ ID NOs. 49-53. In some embodiments, the artificial polypeptide of formula (III) comprises a sequence having at least 95% identity with any one selected from SEQ ID NOs. 49-53. In some embodiments, the artificial polypeptide of formula (III) comprises a sequence having at least 96%, at least 97%, at least 98%, or at least 99% identity with any one selected from SEQ ID NOs. 49-53. In some embodiments, the artificial polypeptide of formula (III) comprises a sequence of any one selected from SEQ ID NOs. 49-53. In some embodiments, the artificial polypeptide of formula (III) is a sequence of any one selected from SEQ ID NOs. 49-53.

In another aspect, provided is an artificial polypeptide of formula X—Y (IV).

In some embodiments, X of formula (IV) is a hydrophilic moiety, at least 50% of which are selected from H, R, K, D, Q, N and E. In some embodiments, X of formula (IV) can be a hydrophilic moiety, at least 55% of which are selected from R, K, N, D, Q, E, and H. In some embodiments, X of formula (IV) can be a hydrophilic moiety, at least 60% of which are selected from R, K, N, D, Q, E, and H. In some embodiments, X of formula (IV) can be a hydrophilic moiety, at least 65% of which are selected from R, K, N, D, Q, E, and H. In some embodiments, X of formula (IV) can be a hydrophilic moiety, at least 70% of which are selected from R, K, N, D, Q, E, and H. In some embodiments, X of formula (IV) can be a hydrophilic moiety, at least 75% of which are selected from R, K, N, D, Q, E, and H. In some embodiments, X of formula (IV) can be a hydrophilic moiety, at least 80% of which are selected from R, K, N, D, Q, E, and H. In some embodiments, X of formula (IV) can be a hydrophilic moiety, at least 85% of which are selected from R, K, N, D, Q, E, and H. In some embodiments, X of formula (IV) can be a hydrophilic moiety, at least 90% of which are selected from R, K, N, D, Q, E, and H.

In some embodiments, X of formula (IV) comprises 40 to 65 amino acids, and a total number of hydrophilic amino acids in X is more than 10. In some embodiments, X of formula (IV) comprises 40 to 65 amino acids, and a total number of hydrophilic amino acids in X is more than 15. In some embodiments, X of formula (IV) comprises 40 to 65 amino acids, and a total number of hydrophilic amino acids in X is more than 20. In some embodiments, X of formula (IV) comprises 40 to 65 amino acids, and a total number of hydrophilic amino acids in X is more than 25. In some embodiments, X of formula (IV) comprises 40 to 65 amino acids, and a total number of hydrophilic amino acids in X is more than 30. In some embodiments, X of formula (IV) comprises 40 to 65 amino acids, and a total number of hydrophilic amino acids in X is more than 35.

In some embodiments, X of formula (IV) comprises a sequence having at most 50% identity with SEQ ID NO. 1. In some embodiments, X of formula (IV) comprises a sequence having at most 55% identity with SEQ ID NO. 1. In some embodiments, X of formula (IV) comprises a sequence having at most 60% identity with SEQ ID NO. 1. In some embodiments, X of formula (IV) comprises a sequence having at most 65% identity with SEQ ID NO. 1. In some embodiments, X of formula (IV) comprises a sequence having at most 70% identity with SEQ ID NO. 1. In some embodiments, X of formula (IV) comprises a sequence having at most 75% identity with SEQ ID NO. 1. In some embodiments, X of formula (IV) comprises a sequence having at most 80% identity with SEQ ID NO. 1. In some embodiments, X of formula (IV) comprises a sequence having at most 85% identity with SEQ ID NO. 1. In some embodiments, X of formula (IV) comprises a sequence having at most 90% identity with SEQ ID NO. 1.

In some embodiments, Y of formula (IV) is a moiety comprising a sequence having at least 70% identity with SEQ ID NO. 2. In some embodiments, Y of formula (IV) is a moiety comprising a sequence having at least 75% identity with SEQ ID NO. 2. In some embodiments, Y of formula (IV) is a moiety comprising a sequence having at least 80% identity with SEQ ID NO. 2. In some embodiments, Y of formula (IV) is a moiety comprising a sequence having at least 85% identity with SEQ ID NO. 2. In some embodiments, Y of formula (IV) is a moiety comprising a sequence having at least 90% identity with SEQ ID NO. 2. In some embodiments, Y of formula (IV) is a moiety comprising a sequence having at least 95% identity with SEQ ID NO, 2. In some embodiments, Y of formula (IV) is a moiety comprising a sequence having at least 96%, at least 97%, at least 98%, or at least 99% identity with SEQ ID NO. 2. In some embodiments, Y of formula (IV) is a moiety comprising a sequence of SEQ ID NO. 2. In some embodiments, Y of formula (IV) is a moiety that is a sequence of SEQ ID NO. 2.

In some embodiments, Y of formula (IV) is hydrophobic moiety, at least 50% amino acids of Y are selected from I, V, L, F, C, M, and A. In some embodiments, Y of formula (IV) is hydrophobic moiety, at least 55% amino acids of Y are selected from I, V, L, F, C, M, and A. In some embodiments, Y of formula (IV) is hydrophobic moiety, at least 60% amino acids of Y are selected from I, V, L, F, C, M, and A. In some embodiments, Y of formula (IV) is hydrophobic moiety, at least 65% amino acids of Y are selected from I, V, L, F, C, M, and A. In some embodiments, Y of formula (IV) is hydrophobic moiety, at least 70% amino acids of Y are selected from I, V, L, F, C, M, and A. In some embodiments, Y of formula (IV) is hydrophobic moiety, at least 75% amino acids of Y are selected from I, V, L, F, C, M, and A. In some embodiments, Y of formula (IV) is hydrophobic moiety, at least 80% amino acids of Y are selected from I, V, L, F, C, M, and A. In some embodiments, Y of formula (IV) is hydrophobic moiety, at least 85% amino acids of Y are selected from I, V, L, F, C, M, and A. In some embodiments, Y of formula (IV) is hydrophobic moiety, at least 90% amino acids of Y are selected from I, V, L, F, C, M, and A.

In some embodiments, X of formula (IV) comprises a sequence having at least 70% identity with any one selected from SEQ IDs NO. 19-22 and 76-79. In some embodiments, X of formula (IV) comprises a sequence having at least 75% identity with any one selected from SEQ IDs NO. 19-22 and 76-79. In some embodiments, X of formula (IV) comprises a sequence having at least 80% identity with any one selected from SEQ IDs NO. 19-22 and 76-79. In some embodiments, X of formula (IV) comprises a sequence having at least 85% identity with any one selected from SEQ IDs NO. 19-22 and 76-79. In some embodiments, X of formula (IV) comprises a sequence having at least 90% identity with any one selected from SEQ IDs NO. 19-22 and 76-79. In some embodiments, X of formula (IV) comprises a sequence having at least 95% identity with any one selected from SEQ IDs NO. 19-22 and 76-79. In some embodiments, X of formula (IV) comprises a sequence having at least 96%, at least 97%, at least 98%, or at least 99% identity with any one selected from SEQ IDs NO. 19-22 and 76-79. In some embodiments, X of formula (IV) comprises a sequence of any one selected from SEQ IDs NO. 19-22 and 76-79. In some embodiments, X of formula (IV) is a sequence of any one selected from SEQ IDs NO. 19-22 and 76-79.

In some embodiments, the artificial polypeptide of formula (IV) comprises a sequence having at least 70% identity with any one selected from SEQ ID NOs. 45-48 and 86-89. In some embodiments, the artificial polypeptide of formula (IV) comprises a sequence having at least 75% identity with any one selected from SEQ ID NOs. 45-48 and 86-89. In some embodiments, the artificial polypeptide of formula (IV) comprises a sequence having at least 80% identity with any one selected from SEQ ID NOs. 45-48 and 86-89. In some embodiments, the artificial polypeptide of formula (IV) comprises a sequence having at least 85% identity with any one selected from SEQ ID NOs. 45-48 and 86-89. In some embodiments, the artificial polypeptide of formula (IV) comprises a sequence having at least 90% identity with any one selected from SEQ ID NOs. 45-48 and 86-89. In some embodiments, the artificial polypeptide of formula (IV) comprises a sequence having at least 95% identity with any one selected from SEQ ID NOs. 45-48 and 86-89. In some embodiments, the artificial polypeptide of formula (IV) comprises a sequence having at least 96%, at least 97%, at least 98%, or at least 99% identity with any one selected from SEQ ID NOs. 45-48 and 86-89. In some embodiments, the artificial polypeptide of formula (IV) comprises a sequence of any one selected from SEQ ID NOs. 45-48 and 86-89. In some embodiments, the artificial polypeptide of formula (IV) is a sequence of any one selected from SEQ ID NOs. 45-48 and 86-89.

In another aspect, provided is an artificial polypeptide of formula (V): X—Y (V), wherein X is a moiety comprising a sequence having at least 80% identity with SEQ ID NO. 1, and Y is a moiety comprising 10 to 30 amino acids, wherein Y comprises a sequence having at least 10 continuous AAs of SEQ ID NO: 2.

In some embodiments, Y of formula (V) comprises 10 to 25 amino acids. In some embodiments, Y comprises 10 to 20 amino acids. In some embodiments, Y comprises 10 to 15 amino acids. In some embodiments, Y comprises 10, 11, 12, 13, 14, 15, 16, 17, 18, 19, 20, 21, 22, 23, 24, 25, 26, 27, 28, 29 or 30 amino acids.

In some embodiments, X of formula (V) can be a moiety comprising a sequence having at least 80% identity with SEQ ID NO. 1. In some embodiments, X is a moiety comprising a sequence having at least 85% identity with SEQ ID NO. 1. In some embodiments, X is a moiety comprising a sequence having at least 90% identity with SEQ ID NO. 1. In some embodiments, X is a moiety comprising a sequence having at least 95% identity with SEQ ID NO. 1. In some embodiments, X is a moiety comprising a sequence having at least 96%, at least 97%, at least 98%, or at least 99% identity with SEQ ID NO. 1. In some embodiments, X is a moiety comprising the sequence of SEQ ID NO. 1. In some embodiments, X is a moiety that is the sequence of SEQ ID NO. 1.

In some embodiments of the artificial polypeptide of formula (V), at least 50% amino acids of X are selected from R, K, N, D, Q, E, and H. In some embodiments, at least 55% amino acids of X are selected from R, K, N, D, Q, E, and H. In some embodiments, at least 60% amino acids of X are selected from R, K, N, D, Q, E, and H. In some embodiments, at least 65% amino acids of X are selected from R, K, N, D, Q, E, and H. In some embodiments, at least 70% amino acids of X are selected from R, K, N, D, Q, E, and H. In some embodiments, at least 75% amino acids of X are selected from R, K, N, D, Q, E, and H. In some embodiments, at least 80% amino acids of X are selected from R, K, N, D, Q, E, and H. In some embodiments, at least 85% amino acids of X are selected from R, K, N, D, Q, E, and H. In some embodiments, at least 90% amino acids of X are selected from R, K, N, D, Q, E, and H.

In some embodiments of the artificial polypeptide of formula (V), Y comprises a sequence having at most 50% identity with SEQ ID NO. 2. In some embodiments, Y comprises a sequence having at most 55% identity with SEQ ID NO. 2. In some embodiments, Y comprises a sequence having at most 60% identity with SEQ ID NO. 2. In some embodiments, Y comprises a sequence having at most 65% identity with SEQ ID NO, 2. In some embodiments, Y comprises a sequence having at most 70% identity with SEQ ID NO. 2. In some embodiments, Y comprises a sequence having at most 75% identity with SEQ ID NO. 2. In some embodiments, Y comprises a sequence having at most 80% identity with SEQ ID NO. 2. In some embodiments, Y comprises a sequence having at most 85% identity with SEQ ID NO. 2. In some embodiments, Y comprises a sequence having at most 90% identity with SEQ ID NO. 2.

In some embodiments, the artificial polypeptide of formula (V) comprises a sequence having at least 70% identity with any one selected from SEQ ID NOs. 53-56. In some embodiments, the artificial polypeptide of formula (V) comprises a sequence having at least 75% identity with any one selected from SEQ ID NOs. 53-56. In some embodiments, the artificial polypeptide of formula (V) comprises a sequence having at least 80% identity with any one selected from SEQ ID NOs. 53-56. In some embodiments, the artificial polypeptide of formula (V) comprises a sequence having at least 85% identity with any one selected from SEQ ID NOs. 53-56. In some embodiments, the artificial polypeptide of formula (V) comprises a sequence having at least 90% identity with any one selected from SEQ ID NOs. 53-56. In some embodiments, the artificial polypeptide of formula (V) comprises a sequence having at least 95% identity with any one selected from SEQ ID NOs. 53-56. In some embodiments, the artificial polypeptide of formula (V) comprises a sequence having at least 96%, at least 97%, at least 98%, or at least 99% identity with any one selected from SEQ ID NOs. 53-56. In some embodiments, the artificial polypeptide of formula (V) comprises a sequence of any one selected from SEQ ID NOs. 53-56. In some embodiments, the artificial polypeptide of formula (V) is a sequence of any one selected from SEQ ID NOs. 53-56.

In another aspect, provided is an artificial polypeptide of formula X—Y (VI).

In some embodiments, X of formula (VI) comprises a sequence having at least 70% identity with SEQ ID NO. 1. In some embodiments, X of formula (VI) comprises a sequence having at least 75% identity with SEQ ID NO. 1. In some embodiments, X of formula (VI) comprises a sequence having at least 80% identity with SEQ ID NO. 1. In some embodiments, X of formula (VI) comprises a sequence having at least 85% identity with SEQ ID NO. 1. In some embodiments, X of formula (VI) comprises a sequence having at least 90% identity with SEQ ID NO. 1. In some embodiments, X of formula (VI) comprises a sequence having at least 95% identity with SEQ ID NO. 1. In some embodiments, X of formula (VI) comprises a sequence having at most 95% identity with SEQ ID NO. 1. In some embodiments, X of formula (VI) comprises a sequence having at most 90% identity with SEQ ID NO. 1. In some embodiments, X of formula (VI) comprises a sequence having at most 85% identity with SEQ ID NO. 1. In some embodiments, X of formula (VI) comprises a sequence having at most 80% identity with SEQ ID NO. 1. In some embodiments, X of formula (VI) comprises a sequence having at most 75% identity with SEQ ID NO. 1.

In some embodiments, Y of formula (VI) is a moiety comprising a sequence having at least 70% identity with SEQ ID NO. 2. In some embodiments, Y of formula (VI) is a moiety comprising a sequence having at least 75% identity with SEQ ID NO. 2. In some embodiments, Y of formula (VI) is a moiety comprising a sequence having at least 80% identity with SEQ ID NO. 2. In some embodiments, Y of formula (VI) is a moiety comprising a sequence having at least 85% identity with SEQ ID NO. 2. In some embodiments, Y of formula (VI) is a moiety comprising a sequence having at least 90% identity with SEQ ID NO. 2. In some embodiments, Y of formula (VI) is a moiety comprising a sequence having at least 95% identity with SEQ ID NO, 2. In some embodiments, Y of formula (VI) comprises a sequence having at most 95% identity with SEQ ID NO. 2. In some embodiments, Y of formula (VI) comprises a sequence having at most 90% identity with SEQ ID NO. 2. In some embodiments, Y of formula (VI) comprises a sequence having at most 85% identity with SEQ ID NO. 2. In some embodiments, Y of formula (VI) comprises a sequence having at most 80%-identity with SEQ ID NO. 2. In some embodiments, Y of formula (VI) comprises a sequence having at most 75% identity with SEQ ID NO. 2.

In some embodiments, Y of formula (VI) is hydrophobic moiety, at least 50% amino acids of Y are selected from I, V, L, F, C, M, and A. In some embodiments, Y of formula (VI) is hydrophobic moiety, at least 55% amino acids of Y are selected from I, V, L, F, C, M, and A. In some embodiments, Y of formula (VI) is hydrophobic moiety, at least 60% amino acids of Y are selected from I, V, L, F, C, M, and A. In some embodiments, Y of formula (VI) is hydrophobic moiety, at least 65% amino acids of Y are selected from I, V, L, F, C, M, and A. In some embodiments, Y of formula (VI) is hydrophobic moiety, at least 70% amino acids of Y are selected from I, V, L, F, C, M, and A. In some embodiments, Y of formula (VI) is hydrophobic moiety, at least 75% amino acids of Y are selected from I, V, L, F, C, M, and A. In some embodiments, Y of formula (VI) is hydrophobic moiety, at least 80% amino acids of Y are selected from I, V, L, F, C, M, and A. In some embodiments, Y of formula (VI) is hydrophobic moiety, at least 85% amino acids of Y are selected from I, V, L, F, C, M, and A. In some embodiments, Y of formula (VI) is hydrophobic moiety, at least 90% amino acids of Y are selected from I, V, L, F, C, M, and A.

In some embodiments, X of formula (VI) is a moiety comprising 40 to 65 amino acids, and a total number of R, K, T, A, N, Q, D, E, S, and G in X is more than 30. In some embodiments, X of formula (VI) is a moiety comprising 40 to 65 amino acids, and a total number of R, K, T, A, N, Q, D, E, S, and G in X is 31, 32, 33, 34, 35, 36, 37, 38, 39 or 40. In some embodiments, X of formula (VI) is a moiety comprising 40 to 65 amino acids, and a total number of W, Y, F, M, L, I, and V in X is no more than 20. In some embodiments, X of formula (VI) is a moiety comprising 40 to 65 amino acids, and a total number of W, Y, F, M, L, I, and V in X is 19, 18, 17, 16, 15 or 14.

In some embodiments, Y of formula (VI) is a moiety comprising 10 to 50 amino acids, and a total number of R, K, T, A, N, Q, D, E, S, and G in Y is more than 10. In some embodiments, Y of formula (VI) is a moiety comprising 10 to 50 amino acids, and a total number of R, K, T, A, N, Q, D, E, S, and G in Y is 11, 12, 13, 14 or 15. In some embodiments, Y of formula (VI) is a moiety comprising 10 to 50 amino acids, and a total number of W, Y, F, M, L, I, and V in X is no more than 20. In some embodiments, Y of formula (VI) is a moiety comprising 10 to 50 amino acids, and a total number of W, Y, F, M, L, I, and V in Y is 19, 18, 17, 16, 15 or 14.

In some embodiments, X of formula (VI) comprises a sequence having at least 70% identity with any one selected from SEQ ID NOs. 80-83. In some embodiments, X of formula (VI) comprises a sequence having at least 75% identity with any one selected from SEQ ID NOs. 80-83. In some embodiments, X of formula (VI) comprises a sequence having at least 80% identity with any one selected from SEQ ID NOs. 80-83. In some embodiments, X of formula (VI) comprises a sequence having at least 85% identity with any one selected from SEQ ID NOs. 80-83. In some embodiments, X of formula (VI) comprises a sequence having at least 90% identity with any one selected from SEQ ID NOs. 80-83. In some embodiments, X of formula (VI) comprises a sequence having at least 95% identity with any one selected from SEQ ID NOs. 80-83. In some embodiments, X of formula (VI) comprises a sequence having at least 96%, at least 97%, at least 98%, or at least 99% identity with any one selected from SEQ ID NOs. 80-83. In some embodiments, X of formula (VI) comprises a sequence of any one selected from SEQ ID NOs. 80-83. In some embodiments, X of formula (VI) is a sequence of any one selected from SEQ ID NOs. 80-83.

In some embodiments, Y of formula (VI) comprises a sequence having at least 70% identity with SEQ ID NO. 3. In some embodiments, Y of formula (VI) comprises a sequence having at least 75% identity with SEQ ID NO. 3. In some embodiments, Y of formula (VI) comprises a sequence having at least 80% identity with SEQ ID NO. 3. In some embodiments, Y of formula (VI) comprises a sequence having at least 85% identity with SEQ ID NO. 3. In some embodiments, Y of formula (VI) comprises a sequence having at least 90% identity with SEQ ID NO. 3. In some embodiments, Y of formula (VI) comprises a sequence having at least 95% identity with SEQ ID NO. 3. In some embodiments, Y of formula (VI) comprises a sequence having at least 96%, at least 97%, at least 98%, or at least 99% identity with SEQ ID NO. 3. In some embodiments, Y of formula (VI) comprises a sequence of SEQ ID NO. 3. In some embodiments, Y of formula (VI) is a sequence of SEQ ID NO. 3.

In some embodiments, the artificial polypeptide of formula (VI) comprises a sequence having at least 70% identity with any one selected from SEQ ID NOs. 90-93. In some embodiments, the artificial polypeptide of formula (VI) comprises a sequence having at least 75% identity with any one selected from SEQ ID NOs. 90-93. In some embodiments, the artificial polypeptide of formula (VI) comprises a sequence having at least 80% identity with any one selected from SEQ ID NOs. 90-93. In some embodiments, the artificial polypeptide of formula (VI) comprises a sequence having at least 85% identity with any one selected from SEQ ID NOs. 90-93. In some embodiments, the artificial polypeptide of formula (VI) comprises a sequence having at least 90% identity with any one selected from SEQ ID NOs. 90-93. In some embodiments, the artificial polypeptide of formula (VI) comprises a sequence having at least 95% identity with any one selected from SEQ ID NOs. 90-93. In some embodiments, the artificial polypeptide of formula (VI) comprises a sequence having at least 96%, at least 97%, at least 98%, or at least 99% identity with any one selected from SEQ ID NOs. 90-93. In some embodiments, the artificial polypeptide of formula (VI) comprises a sequence that is any one selected from SEQ ID NOs. 90-93. In some embodiments, the artificial polypeptide of formula (VI) is a sequence that is any one selected from SEQ ID NOs. 90-93.

In another aspect, provided is a mutant of artificial polypeptide of formula (I).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q. E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant comprises a sequence having at least 70% identity with SEQ ID NO. 1. In some embodiments, X in the mutant comprises a sequence having at least 75% identity with SEQ ID NO. 1. In some embodiments, X in the mutant comprises a sequence having at least 80% identity with SEQ ID NO. 1. In some embodiments, X in the mutant comprises a sequence having at least 85% identity with SEQ ID NO. 1. In some embodiments, X in the mutant comprises a sequence having at least 90% identity with SEQ ID NO. 1. In some embodiments, X in the mutant comprises a sequence having at least 95% identity with SEQ ID NO. 1.

In some embodiments, Y in the mutant can be a moiety comprising 10 to 50 amino acids, and a total number of Cysteine (C) of Y is 5. In some embodiments, Y in the mutant can be a moiety comprising 10 to 50 amino acids, and a total number of Cysteine (C) of Y is less than 5. In some embodiments, Y in the mutant can be a moiety comprising 10 to 50 amino acids, and a total number of Cysteine (C) of Y is 4. In some embodiments, Y in the mutant can be a moiety comprising 10 to 50 amino acids, and a total number of Cysteine (C) of Y is less than 4. In some embodiments, Y in the mutant can be a moiety comprising 10 to 50 amino acids, and a total number of Cysteine (C) of Y is 3. In some embodiments, Y in the mutant can be a moiety comprising 10 to 50 amino acids, and a total number of Cysteine (C) of Y is less than 3. In some embodiments, Y in the mutant can be a moiety comprising 10 to 50 amino acids, and a total number of Cysteine (C) of Y is 2. In some embodiments, Y in the mutant can be a moiety comprising 10 to 50 amino acids, and a total number of Cysteine (C) of Y is less than 2. In some embodiments, Y in the mutant can be a moiety comprising 10 to 50 amino acids, and a total number of Cysteine (C) of Y is 1. In some embodiments, Y in the mutant can be a moiety comprising 10 to 50 amino acids, and a total number of Cysteine (C) of Y is less than 1. In some embodiments, Y in the mutant can be a moiety comprising 10 to 50 amino acids, and a total number of Cysteine (C) of Y is 0.

In some embodiments, a total number of hydrophobic amino acids in Y in the mutant is more than 8. In some embodiments, a total number of hydrophobic amino acids in Y in the mutant is more than 9. In some embodiments, a total number of hydrophobic amino acids in Y in the mutant is more than 10. In some embodiments, a total number of hydrophobic amino acids in Y in the mutant is more than 11. In some embodiments, the total number of hydrophobic amino acids in Y in the mutant is more than 12. In some embodiments, the total number of hydrophobic amino acids in Y in the mutant is more than 13. In some embodiments, the total number of hydrophobic amino acids in Y in the mutant is more than 14. In some embodiments, the total number of hydrophobic amino acids in Y in the mutant is more than 15.

In some embodiments, a total number of hydrophilic amino acids in Y in the mutant is 5. In some embodiments, a total number of hydrophilic amino acids in Y in the mutant is no more than 5. In some embodiments, a total number of hydrophilic amino acids in Y in the mutant is 4. In some embodiments, a total number of hydrophilic amino acids in Y in the mutant is no more than 4. In some embodiments, a total number of hydrophilic amino acids in Y in the mutant is 3. In some embodiments, a total number of hydrophilic amino acids in Y in the mutant is no more than 3. In some embodiments, a total number of hydrophilic amino acids in Y in the mutant is 2. In some embodiments, a total number of hydrophilic amino acids in Y in the mutant is no more than 2. In some embodiments, a total number of hydrophilic amino acids in Y in the mutant is 1. In some embodiments, a total number of hydrophilic amino acids in Y in the mutant is no more than 1.

In some embodiments, Y in the mutant comprises a sequence having at least 70% identity with any one selected from SEQ ID NOs. 3-15, 58-61 and 84-85. In some embodiments, Y in the mutant comprises a sequence having at least 75% identity with any one selected from SEQ ID NOs. 3-15, 58-61 and 84-85. In some embodiments, Y in the mutant comprises a sequence having at least 80% identity with any one selected from SEQ ID NOs. 3-15, 58-61 and 84-85. In some embodiments, Y in the mutant comprises a sequence having at least 85% identity with any one selected from SEQ ID NOs. 3-15, 58-61 and 84-85. In some embodiments, Y in the mutant comprises a sequence having at least 90% identity with any one selected from SEQ ID NOs. 3-15, 58-61 and 84-85. In some embodiments, Y in the mutant comprises a sequence having at least 95% identity with any one selected from SEQ ID NOs. 3-15, 58-61 and 84-85. In some embodiments, Y in the mutant comprises a sequence having at least 96%, at least 97%, at least 98%, or at least 99% identity with any one selected from SEQ ID NOs. 3-15, 58-61 and 84-85. In some embodiments, Y in the mutant comprises a sequence of any one selected from SEQ ID NOs. 3-15, 58-61 and 84-85. In some embodiments, Y in the mutant is a sequence of any one selected from SEQ ID NOs. 3-15, 58-61 and 84-85.

In some embodiments, Y in the mutant comprises a sequence having at least 70% identity with any one selected from SEQ ID NOs. 3-18, 58-61 and 84-85. In some embodiments, Y in the mutant comprises a sequence having at least 75% identity with any one selected from SEQ ID NOs. 3-18, 58-61 and 84-85. In some embodiments, Y in the mutant comprises a sequence having at least 80% identity with any one selected from SEQ ID NOs. 3-18, 58-61 and 84-85. In some embodiments, Y in the mutant comprises a sequence having at least 85% identity with any one selected from SEQ ID NOs. 3-18, 58-61 and 84-85. In some embodiments, Y in the mutant comprises a sequence having at least 90% identity with any one selected from SEQ ID NOs. 3-18, 58-61 and 84-85. In some embodiments, Y in the mutant comprises a sequence having at least 95% identity with any one selected from SEQ ID NOs. 3-18, 58-61 and 84-85. In some embodiments, Y in the mutant comprises a sequence having at least 96%, at least 97%, at least 98%, or at least 99% identity with any one selected from SEQ ID NOs. 3-18, 58-61 and 84-85. In some embodiments, Y in the mutant comprises a sequence of any one selected from SEQ ID NOs. 3-18, 58-61 and 84-85. In some embodiments, Y in the mutant is a sequence of any one selected from SEQ ID NOs. 3-18, 58-61 and 84-85.

In another aspect, provided is a mutant of artificial polypeptide of formula (II).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant comprises a sequence having at least 70% identity with SEQ ID NO. 1. In some embodiments, X in the mutant comprises a sequence having at least 75% identity with SEQ ID NO. 1. In some embodiments, X in the mutant comprises a sequence having at least 80% identity with SEQ ID NO. 1. In some embodiments, X in the mutant comprises a sequence having at least 85% identity with SEQ ID NO. 1. In some embodiments, X in the mutant comprises a sequence having at least 90% identity with SEQ ID NO. 1. In some embodiments, X in the mutant comprises a sequence having at least 95% identity with SEQ ID NO. 1.

In some embodiments, Y in the mutant comprises a sequence having at most 50% identity with SEQ ID NO. 2. In some embodiments, Y in the mutant comprises a sequence having at most 55% identity with SEQ ID NO. 2. In some embodiments, Y in the mutant comprises a sequence having at most 60% identity with SEQ ID NO. 2. In some embodiments, Y in the mutant comprises a sequence having at most 65% identity with SEQ ID NO. 2. In some embodiments, Y in the mutant comprises a sequence having at most 70% identity with SEQ ID NO. 2. In some embodiments, Y in the mutant comprises a sequence having at most 75% identity with SEQ ID NO. 2. In some embodiments, Y in the mutant comprises a sequence having at most 80% identity with SEQ ID NO. 2. In some embodiments, Y in the mutant comprises a sequence having at most 85% identity with SEQ ID NO. 2. In some embodiments, Y in the mutant comprises a sequence having at most 90% identity with SEQ ID NO. 2.

In some embodiments, a total number of hydrophobic amino acids in Y in the mutant is more than 8. In some embodiments, a total number of hydrophobic amino acids in Y in the mutant is more than 9. In some embodiments, a total number of hydrophobic amino acids in Y in the mutant is more than 10. In some embodiments, a total number of hydrophobic amino acids in Y in the mutant is more than 11. In some embodiments, the total number of hydrophobic amino acids in Y in the mutant is more than 12. In some embodiments, the total number of hydrophobic amino acids in Y in the mutant is more than 13. In some embodiments, the total number of hydrophobic amino acids in Y in the mutant is more than 14. In some embodiments, the total number of hydrophobic amino acids in Y in the mutant is more than 15.

In some embodiments, a total number of hydrophilic amino acids in Y in the mutant is 5. In some embodiments, a total number of hydrophilic amino acids in Y in the mutant is no more than 5. In some embodiments, a total number of hydrophilic amino acids in Y in the mutant is 4. In some embodiments, a total number of hydrophilic amino acids in Y in the mutant is no more than 4. In some embodiments, a total number of hydrophilic amino acids in Y in the mutant is 3. In some embodiments, a total number of hydrophilic amino acids in Y in the mutant is no more than 3. In some embodiments, a total number of hydrophilic amino acids in Y in the mutant is 2. In some embodiments, a total number of hydrophilic amino acids in Y in the mutant is no more than 2. In some embodiments, a total number of hydrophilic amino acids in Y in the mutant is 1. In some embodiments, a total number of hydrophilic amino acids in Y in the mutant is no more than 1.

In some embodiments, Y in the mutant comprises a sequence having at least 70% identity with any one selected from SEQ ID NOs. 16-18. In some embodiments, Y in the mutant comprises a sequence having at least 75% identity with any one selected from SEQ ID NOs. 16-18. In some embodiments, Y in the mutant comprises a sequence having at least 80% identity with any one selected from SEQ ID NOs. 16-18. In some embodiments, Y in the mutant comprises a sequence having at least 85% identity with any one selected from SEQ ID NOs. 16-18. In some embodiments, Y in the mutant comprises a sequence having at least 90% identity with any one selected from SEQ ID NOs. 16-18. In some embodiments, Y in the mutant comprises a sequence having at least 95% identity with any one selected from SEQ ID NOs. 16-18. In some embodiments, Y in the mutant comprises a sequence having at least 96%, at least 97%, at least 98%, or at least 99% identity with any one selected from SEQ ID NOs. 16-18. In some embodiments, Y in the mutant comprises a sequence of any one selected from SEQ ID NOs. 16-18. In some embodiments, Y in the mutant is a sequence of any one selected from SEQ ID NOs. 16-18.

In another aspect, provided is a mutant of artificial polypeptide of formula (III).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant is a moiety comprising 40 to 65 amino acids, and a total number of H, R, K, D, Q, N and E in X is less than 33. In some embodiments, X in the mutant is a moiety comprising 40 to 65 amino acids, and a total number of H, R, K, D, Q, N and E in X is less than 32. In some embodiments, X in the mutant is a moiety comprising 40 to 65 amino acids, and a total number of H, R, K, D, Q, N and E in X is less than 31. In some embodiments, X in the mutant is a moiety comprising 40 to 65 amino acids, and a total number of H, R, K, D, Q, N and E in X is less than 30. In some embodiments, X in the mutant is a moiety comprising 40 to 65 amino acids, and a total number of H, R, K, D, Q, N and E in X is less than 29. In some embodiments, X in the mutant is a moiety comprising 40 to 65 amino acids, and a total number of H, R, K, D, Q, N and E in X is less than 28. In some embodiments, X in the mutant is a moiety comprising 40 to 65 amino acids, and a total number of H, R, K, D, Q, N and E in X is less than 27. In some embodiments, X in the mutant is a moiety comprising 40 to 65 amino acids, and a total number of H, R, K, D, Q, N and E in X is less than 26. In some embodiments, X in the mutant is a moiety comprising 40 to 65 amino acids, and a total number of H, R, K, D, Q, N and E in X is less than 25. In some embodiments, X in the mutant is a moiety comprising 40 to 65 amino acids, and a total number of H, R, K, D, Q, N and E in X is less than 24. In some embodiments, X in the mutant is a moiety comprising 40 to 65 amino acids, and a total number of H, R, K, D, Q, N and E in X is less than 23. In some embodiments, X in the mutant is a moiety comprising 40 to 65 amino acids, and a total number of H, R, K, D, Q, N and E in X is less than 22. In some embodiments, X in the mutant is a moiety comprising 40 to 65 amino acids, and a total number of H, R, K, D, Q, N and E in X is less than 21. In some embodiments, X in the mutant is a moiety comprising 40 to 65 amino acids, and a total number of H, R, K, D, Q, N and E in X is less than 20.

In some embodiments, X in the mutant is a moiety comprising 40 to 65 amino acids, and a total number of hydrophilic amino acids in X is more than 10. In some embodiments, X in the mutant is a moiety comprising 40 to 65 amino acids, and a total number of hydrophilic amino acids in X is more than 11. In some embodiments, X in the mutant is a moiety comprising 40 to 65 amino acids, and a total number of hydrophilic amino acids in X is more than 12. In some embodiments, X in the mutant is a moiety comprising 40 to 65 amino acids, and a total number of hydrophilic amino acids in X is more than 13. In some embodiments, X in the mutant is a moiety comprising 40 to 65 amino acids, and a total number of hydrophilic amino acids in X is more than 14. In some embodiments, X in the mutant is a moiety comprising 40 to 65 amino acids, and a total number of hydrophilic amino acids in X is more than 15. In some embodiments, X in the mutant is a moiety comprising 40 to 65 amino acids, and a total number of hydrophilic amino acids in X is more than 16. In some embodiments, X in the mutant is a moiety comprising 40 to 65 amino acids, and a total number of hydrophilic amino acids in X is more than 17. In some embodiments, X in the mutant is a moiety comprising 40 to 65 amino acids, and a total number of hydrophilic amino acids in X is more than 18. In some embodiments, X in the mutant is a moiety comprising 40 to 65 amino acids, and a total number of hydrophilic amino acids in X is more than 19. In some embodiments, X in the mutant is a moiety comprising 40 to 65 amino acids, and a total number of hydrophilic amino acids in X is more than 20. In some embodiments, X in the mutant is a moiety comprising 40 to 65 amino acids, and a total number of hydrophilic amino acids in X is more than 21. In some embodiments, X in the mutant is a moiety comprising 40 to 65 amino acids, and a total number of hydrophilic amino acids in X is more than 22. In some embodiments, X in the mutant is a moiety comprising 40 to 65 amino acids, and a total number of hydrophilic amino acids in X is more than 23. In some embodiments, X in the mutant is a moiety comprising 40 to 65 amino acids, and a total number of hydrophilic amino acids in X is more than 24. In some embodiments, X in the mutant is a moiety comprising 40 to 65 amino acids, and a total number of hydrophilic amino acids in X is more than 25.

In some embodiments, X in the mutant is a moiety comprising 40 to 65 amino acids, and a total number of hydrophobic amino acids in X is no more than 15. In some embodiments, X in the mutant is a moiety comprising 40 to 65 amino acids, and a total number of hydrophobic amino acids in X is no more than 14. In some embodiments, X in the mutant is a moiety comprising 40 to 65 amino acids, and a total number of hydrophobic amino acids in X is no more than 13. In some embodiments, X in the mutant is a moiety comprising 40 to 65 amino acids, and a total number of hydrophobic amino acids in X is no more than 12. In some embodiments, X in the mutant is a moiety comprising 40 to 65 amino acids, and a total number of hydrophobic amino acids in X is no more than 11. In some embodiments, X in the mutant is a moiety comprising 40 to 65 amino acids, and a total number of hydrophobic amino acids in X is no more than 10. In some embodiments, X in the mutant is a moiety comprising 40 to 65 amino acids, and a total number of hydrophobic amino acids in X is no more than 5.

In some embodiments, X in the mutant comprises a sequence having at least 70% identity with SEQ ID NO. 1. In some embodiments, X in the mutant comprises a sequence having at least 75% identity with SEQ ID NO. 1. In some embodiments, X in the mutant comprises a sequence having at least 80% identity with SEQ ID NO. 1. In some embodiments, X in the mutant comprises a sequence having at least 85% identity with SEQ ID NO. 1. In some embodiments, X in the mutant comprises a sequence having at least 90% identity with SEQ ID NO. 1. In some embodiments, X in the mutant comprises a sequence having at least 95% identity with SEQ ID NO. 1.

In some embodiments, Y in the mutant is a moiety comprising a sequence having at least 70% identity with SEQ ID NO. 2. In some embodiments, Y in the mutant is a moiety comprising a sequence having at least 75% identity with SEQ ID NO. 2. In some embodiments, Y in the mutant is a moiety comprising a sequence having at least 80% identity with SEQ ID NO. 2. In some embodiments, Y in the mutant is a moiety comprising a sequence having at least 85% identity with SEQ ID NO. 2. In some embodiments, Y in the mutant is a moiety comprising a sequence having at least 90% identity with SEQ ID NO. 2. In some embodiments, Y in the mutant is a moiety comprising a sequence having at least 95% identity with SEQ ID NO. 2. In some embodiments, Y in the mutant is a moiety comprising a sequence having at least 96%, at least 97%, at least 98%, or at least 99% identity with SEQ ID NO. 2. In some embodiments, Y in the mutant is a moiety comprising a sequence of SEQ ID NO. 2. In some embodiments, Y in the mutant is a moiety that is a sequence of SEQ ID NO. 2.

In some embodiments, Y in the mutant is hydrophobic moiety, at least 50% amino acids of Y are selected from I, V, L, F, C, M, and A. In some embodiments, Y in the mutant is hydrophobic moiety, at least 55% amino acids of Y are selected from I, V, L, F, C, M, and A. In some embodiments, Y in the mutant is hydrophobic moiety, at least 60% amino acids of Y are selected from I, V, L, F, C, M, and A. In some embodiments, Y in the mutant is hydrophobic moiety, at least 65% amino acids of Y are selected from I, V, L, F, C, M, and A. In some embodiments, Y in the mutant is hydrophobic moiety, at least 70% amino acids of Y are selected from I, V, L, F, C, M, and A. In some embodiments, Y in the mutant is hydrophobic moiety, at least 75% amino acids of Y are selected from I, V, L, F, C, M, and A. In some embodiments, Y in the mutant is hydrophobic moiety, at least 80% amino acids of Y are selected from I, V, L, F, C, M, and A. In some embodiments, Y in the mutant is hydrophobic moiety, at least 85% amino acids of Y are selected from I, V, L, F, C, M, and A. In some embodiments, Y in the mutant is hydrophobic moiety, at least 90% amino acids of Y are selected from I, V, L, F, C, M, and A.

In another aspect, provided is a mutant of artificial polypeptide of formula (IV).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, X in the mutant comprises 40 to 65 amino acids, and a total number of hydrophilic amino acids in X is more than 10. In some embodiments, X in the mutant comprises 40 to 65 amino acids, and a total number of hydrophilic amino acids in X is more than 15. In some embodiments, X in the mutant comprises 40 to 65 amino acids, and a total number of hydrophilic amino acids in X is more than 20. In some embodiments, X in the mutant comprises 40 to 65 amino acids, and a total number of hydrophilic amino acids in X is more than 25. In some embodiments, X in the mutant comprises 40 to 65 amino acids, and a total number of hydrophilic amino acids in X is more than 30. In some embodiments, X in the mutant comprises 40 to 65 amino acids, and a total number of hydrophilic amino acids in X is more than 35.

In some embodiments, X in the mutant is a moiety comprising 40 to 65 amino acids, and a total number of hydrophobic amino acids in X is no more than 15. In some embodiments, X in the mutant is a moiety comprising 40 to 65 amino acids, and a total number of hydrophobic amino acids in X is no more than 14. In some embodiments, X in the mutant is a moiety comprising 40 to 65 amino acids, and a total number of hydrophobic amino acids in X is no more than 13. In some embodiments, X in the mutant is a moiety comprising 40 to 65 amino acids, and a total number of hydrophobic amino acids in X is no more than 12. In some embodiments, X in the mutant is a moiety comprising 40 to 65 amino acids, and a total number of hydrophobic amino acids in X is no more than 11. In some embodiments, X in the mutant is a moiety comprising 40 to 65 amino acids, and a total number of hydrophobic amino acids in X is no more than 10. In some embodiments, X in the mutant is a moiety comprising 40 to 65 amino acids, and a total number of hydrophobic amino acids in X is no more than 5.

In some embodiments, X in the mutant comprises a sequence having at least 70% identity with SEQ ID NO. 1. In some embodiments, X in the mutant comprises a sequence having at least 75% identity with SEQ ID NO. 1. In some embodiments, X in the mutant comprises a sequence having at least 80% identity with SEQ ID NO. 1. In some embodiments, X in the mutant comprises a sequence having at least 85% identity with SEQ ID NO. 1. In some embodiments, X in the mutant comprises a sequence having at least 90% identity with SEQ ID NO. 1. In some embodiments, X in the mutant comprises a sequence having at least 95% identity with SEQ ID NO. 1.

In some embodiments, Y in the mutant is a moiety comprising a sequence having at least 70% identity with SEQ ID NO. 2. In some embodiments, Y in the mutant is a moiety comprising a sequence having at least 75% identity with SEQ ID NO. 2. In some embodiments, Y in the mutant is a moiety comprising a sequence having at least 80% identity with SEQ ID NO. 2. In some embodiments, Y in the mutant is a moiety comprising a sequence having at least 85% identity with SEQ ID NO. 2. In some embodiments, Y in the mutant is a moiety comprising a sequence having at least 90% identity with SEQ ID NO. 2. In some embodiments, Y in the mutant is a moiety comprising a sequence having at least 95% identity with SEQ ID NO. 2. In some embodiments, Y in the mutant is a moiety comprising a sequence having at least 96%, at least 97%, at least 98%, or at least 99% identity with SEQ ID NO. 2. In some embodiments, Y in the mutant is a moiety comprising a sequence of SEQ ID NO. 2. In some embodiments, Y in the mutant is a moiety that is a sequence of SEQ ID NO. 2.

In some embodiments, Y in the mutant is hydrophobic moiety, at least 50% amino acids of Y are selected from I, V, L, F, C, M, and A. In some embodiments, Y in the mutant is hydrophobic moiety, at least 55% amino acids of Y are selected from I, V, L, F, C, M, and A. In some embodiments, Y in the mutant is hydrophobic moiety, at least 60% amino acids of Y are selected from I, V, L, F, C, M, and A. In some embodiments, Y in the mutant is hydrophobic moiety, at least 65% amino acids of Y are selected from I, V, L, F, C, M, and A. In some embodiments, Y in the mutant is hydrophobic moiety, at least 70% amino acids of Y are selected from I, V, L, F, C, M, and A. In some embodiments, Y in the mutant is hydrophobic moiety, at least 75% amino acids of Y are selected from I, V, L, F, C, M, and A. In some embodiments, Y in the mutant is hydrophobic moiety, at least 80% amino acids of Y are selected from I, V, L, F, C, M, and A. In some embodiments, Y in the mutant is hydrophobic moiety, at least 85% amino acids of Y are selected from I, V, L, F, C, M, and A. In some embodiments, Y in the mutant is hydrophobic moiety, at least 90% amino acids of Y are selected from I, V, L, F, C, M, and A.

In another aspect, provided is a mutant of artificial polypeptide of formula (V).

In some embodiments, at least 20% amino acids of X in the mutant are selected from R, K, N, D, Q, E, and H. In some embodiments, at least 25% amino acids of X in the mutant are selected from R, K, N, D, Q, E, and H. In some embodiments, at least 30% amino acids of X in the mutant are selected from R, K, N, D, Q, E, and H. In some embodiments, at least 35% amino acids of X in the mutant are selected from R, K, N, D, Q, E, and H. In some embodiments, at least 40% amino acids of X in the mutant are selected from R, K, N, D, Q, E, and H. In some embodiments, at least 45% amino acids of X in the mutant are selected from R, K, N, D, Q, E, and H. In some embodiments, at least 50% amino acids of X in the mutant are selected from R, K, N, D, Q, E, and H. In some embodiments, at most 55% amino acids of X in the mutant are selected from R, K, N, D, Q, E, and H. In some embodiments, at most 60% amino acids of X in the mutant are selected from R, K, N, D, Q, E, and H. In some embodiments, at most 65% amino acids of X in the mutant are selected from R, K, N, D, Q, E, and H. In some embodiments, at most 70% amino acids of X in the mutant are selected from R, K, N, D, Q, E, and H. In some embodiments, at most 75% amino acids of X in the mutant are selected from R, K, N, D, Q, E, and H. In some embodiments, at most 80% amino acids of X in the mutant are selected from R, K, N, D, Q, E, and H. In some embodiments, at most 85% amino acids of X in the mutant are selected from R, K, N, D, Q, E, and H. In some embodiments, at most 90% amino acids of X in the mutant are selected from R, K, N, D, Q, E, and H.

In some embodiments, Y in the mutant comprises 10 to 25 amino acids. In some embodiments, Y in the mutant comprises 10 to 20 amino acids. In some embodiments, Y in the mutant comprises 10 to 15 amino acids. In some embodiments, Y in the mutant comprises 10, 11, 12, 13, 14, 15, 16, 17, 18, 19, 20, 21, 22, 23, 24, 25, 26, 27, 28, 29 or 30 amino acids.

In some embodiments, X in the mutant comprises a sequence having at least 70% identity with SEQ ID NO. 1. In some embodiments, X in the mutant comprises a sequence having at least 75% identity with SEQ ID NO. 1. In some embodiments, X in the mutant comprises a sequence having at least 80% identity with SEQ ID NO. 1. In some embodiments, X in the mutant comprises a sequence having at least 85% identity with SEQ ID NO. 1. In some embodiments, X in the mutant comprises a sequence having at least 90% identity with SEQ ID NO. 1. In some embodiments, X in the mutant comprises a sequence having at least 95% identity with SEQ ID NO. 1.

In some embodiments, Y in the mutant comprises a sequence having at most 50% identity with SEQ ID NO. 2. In some embodiments, Y in the mutant comprises a sequence having at most 55% identity with SEQ ID NO. 2. In some embodiments, Y in the mutant comprises a sequence having at most 60% identity with SEQ ID NO. 2. In some embodiments, Y in the mutant comprises a sequence having at most 65% identity with SEQ ID NO. 2. In some embodiments, Y in the mutant comprises a sequence having at most 70% identity with SEQ ID NO. 2. In some embodiments, Y in the mutant comprises a sequence having at most 75% identity with SEQ ID NO. 2. In some embodiments, Y in the mutant comprises a sequence having at most 80% identity with SEQ ID NO. 2. In some embodiments, Y in the mutant comprises a sequence having at most 85% identity with SEQ ID NO. 2. In some embodiments, Y in the mutant comprises a sequence having at most 90% identity with SEQ ID NO. 2.

In some embodiments, the artificial polypeptide or mutant as described in the present disclosure has enhanced stability as compared to a reference polypeptide. In some embodiments, the artificial polypeptide or mutant as described in the present disclosure has enhanced chemical stability as compared to a reference polypeptide. In some embodiments, the reference polypeptide can be any polypeptide selected from SEQ ID NOs, 28 and 62-71.

In some embodiments, the artificial polypeptide or mutant as described in the present disclosure has enhanced stability as compared to any polypeptide selected from SEQ ID NOs. 28 and 62-71. In some embodiments, the stability of the artificial polypeptide or mutant as described in the present disclosure is enhanced by at least 5%, at least 10%, at least 20%, at least 30%, at least 40%, at least 50%, at least 100%, at least 200%, at least 300%, at least 400%, or at least 500% as compared to any polypeptide selected from SEQ ID NOs. 28 and 62-71. In some embodiments, the artificial polypeptide or mutant as described in the present disclosure has enhanced chemical stability as compared to any polypeptide selected from SEQ ID NOs. 28 and 62-71. In some embodiments, the chemical stability of the artificial polypeptide or mutant as described in the present disclosure is enhanced by at least 5%, at least 10%, at least 20%, at least 30%, at least 40%, at least 50%, at least 100%, at least 200%, at least 300%, at least 400%, or at least 500% as compared to any polypeptide selected from SEQ ID NOs. 28 and 62-71.

In some embodiments, the artificial polypeptide or mutant as described in the present disclosure has a longer shelf-life as compared to a reference polypeptide. In some embodiments, the reference polypeptide can be any polypeptide selected from SEQ ID NOs. 28 and 62-71. In some embodiments, the shelf-life of the artificial polypeptide or mutant is at least 1 day, at least 3 days, at least 5 days, at least 1 week, at least 2 weeks, at least 3 weeks, at least 1 month, at least 2 months, at least 3 months, at least 4 months, at least 5 months, at least 6 months, at least 1 year, at least 2 years, at least 3 years longer than a reference polypeptide. In some embodiments, the shelf-life of the artificial polypeptide or mutant is at least 1 day, at least 3 days, at least 5 days, at least 1 week, at least 2 weeks, at least 3 weeks, at least 1 month, at least 2 months, at least 3 months, at least 4 months, at least 5 months, at least 6 months, at least 1 year, at least 2 years, at least 3 years longer than any polypeptide selected from SEQ ID NOs. 28 and 62-71.

In some embodiments, the artificial polypeptide or mutant as described in the present disclosure has comparative or superior activity as compared to a reference polypeptide. In some embodiments, the artificial polypeptide or mutant as described in the present disclosure has comparative or superior activity as compared to any polypeptide selected from SEQ ID NOs. 28 and 62-71.

In some embodiments, the artificial polypeptide or mutant as described in the present disclosure has comparative or superior cellular activity as compared to a reference polypeptide. In some embodiments, the artificial polypeptide or mutant as described in the present disclosure has comparative or superior cellular activity as compared to any polypeptide selected from SEQ ID NOs. 28 and 62-71.

In some embodiments, the artificial polypeptide or mutant as described in the present disclosure has comparative or superior in vivo activity as compared to a reference polypeptide. In some embodiments, the artificial polypeptide or mutant as described in the present disclosure has comparative or superior in vivo activity as compared to any polypeptide selected from SEQ ID NOs. 28 and 62-71.

In some embodiments, the artificial polypeptide or mutant as described in the present disclosure has comparative or superior in vitro activity as compared to a reference polypeptide. In some embodiments, the artificial polypeptide or mutant as described in the present disclosure has comparative or superior in vitro activity as compared to any polypeptide selected from SEQ ID NOs. 28 and 62-71.

In some embodiments, the artificial polypeptide or mutant as described in the present disclosure has a comparative or superior activity as compared to a reference polypeptide in inhibiting/blocking DAMPs (Damage-associated molecular patterns). In some embodiments, the artificial polypeptide or mutant as described in the present disclosure has a comparative or superior activity as compared to any polypeptide selected from SEQ ID NOs. 28 and 62-71 in inhibiting/blocking DAMPs (Damage-associated molecular patterns).

In some embodiments, the artificial polypeptide or mutant as described in the present disclosure has a comparative or superior activity as compared to a reference polypeptide in inhibiting/blocking ROS. In some embodiments, the artificial polypeptide or mutant as described in the present disclosure has a comparative or superior activity as compared to any polypeptide selected from SEQ ID NOs. 28 and 62-71 in inhibiting/blocking ROS.

In some embodiments, the artificial polypeptide or mutant as described in the present disclosure has a comparative or superior activity as compared to a reference polypeptide in inhibiting thermal BSA aggregation, In some embodiments, the artificial polypeptide or mutant as described in the present disclosure has a comparative or superior activity as compared to any polypeptide selected from SEQ ID NOs. 28 and 62-71 in inhibiting thermal BSA aggregation.

In some embodiments, the artificial polypeptide or mutant as described in the present disclosure has a comparative or superior activity as compared to a reference polypeptide in inhibiting cellular ROS level. In some embodiments, the artificial polypeptide or mutant as described in the present disclosure has a comparative or superior activity as compared to any polypeptide selected from SEQ ID NOs. 28 and 62-71 in inhibiting cellular ROS level.

In some embodiments, the artificial polypeptide or mutant as described in the present disclosure has a comparative or superior activity as compared to a reference polypeptide in inhibiting oxLDL uptake. In some embodiments, the artificial polypeptide or mutant as described in the present disclosure has a comparative or superior activity as compared to any polypeptide selected from SEQ ID NOs. 28 and 62-71 in inhibiting oxLDL uptake.

In some embodiments, the artificial polypeptide or mutant as described in the present disclosure has a comparative or superior activity as compared to a reference polypeptide in binding with an oxidized protein. In some embodiments, the artificial polypeptide or mutant as described in the present disclosure has a comparative or superior activity as compared to any polypeptide selected from SEQ ID NOs. 28 and 62-71 in binding with an oxidized protein.

In some embodiments, the artificial polypeptide or mutant as described in the present disclosure has a comparative or superior activity as compared to a reference polypeptide in binding with a cell. In some embodiments, the artificial polypeptide or mutant as described in the present disclosure has a comparative or superior activity as compared to any polypeptide selected from SEQ ID NOs. 28 and 62-71 in binding with a cell.

In some embodiments, the artificial polypeptide or mutant as described in the present disclosure has comparative or superior activity as compared to a reference polypeptide in facilitating cell membrane penetration. In some embodiments, the artificial polypeptide or mutant as described in the present disclosure has comparative or superior activity as compared to any polypeptide selected from SEQ ID NOs. 28 and 62-71 in facilitating cell membrane penetration.

### Preparation of the Polypeptide

The polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof can be prepared by any suitable method, including but not limited to molecular cloning techniques and synthetic procedures. Standard molecular cloning techniques are well known in the art and are described by Sambrook, J., Fritsch, E. F. and Maniatis, T. Molecular Cloning: A Laboratory Manual; Cold Spring Harbor Laboratory Press: Cold Spring Harbor, (1989) (Maniatis) and by T. J. Silhavy, M. L. Bennan, and L. W. Enquist, Experiments with Gene Fusions, Cold Spring Harbor Laboratory, Cold Spring Harbor, N.Y. (1984) and by Ausubel, F. M. et al., Current Protocols in Molecular Biology, pub. by Greene Publishing Assoc, and Wiley-Interscience (1987).

In some embodiments, the polypeptide of the present disclosure is prepared by reference to the fermentation-based manufacturing method as disclosed in Chinese patent application No. 201711320516.4 (Publication No.: CN109913483A), which is herein incorporated by reference in its entirety. In some embodiments, the method for preparing the polypeptide of the present disclosure comprises the steps of: integrating a target gene fragment into an expression plasmid by means of genetic engineering, with the integrated target gene fragment comprising at least one purification tag; transforming the expression plasmid into a corresponding expression host to construct a recombinant engineered cell which highly expresses the target polypeptide; subjecting the recombinant engineered cell to fermentation, induced expression, and then crude purification to obtain a crude polypeptide; subjecting the crude polypeptide to refined purification to obtain highly purified polypeptide.

In some embodiments, the target gene fragment is any one selected from the group consisting of gene fragments which are capable of encoding the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof. In some embodiments, the target gene fragment is a gene fragment which is capable of encoding an artificial polypeptide of formula (I), (II), (III), (IV), (V) or (VI). In some embodiments, the target gene fragment is a gene fragment which is capable of encoding a mutant of an artificial polypeptide of formula (I), (II), (III), (IV) or (V). In some embodiments, the target gene fragment is a gene fragment which is capable of encoding a polypeptide having at least 70% identity (e.g., at least 80% identity) with any one selected from SEQ ID NO. 28, SEQ ID NOs. 57 and 62-74 or a fragment or variant thereof. The target gene fragment can be prepared by any suitable method, including but not limited to enzymatic synthesis, i.e., using RNA as a template to synthesize cDNA by reverse transcription, and chemical synthesis method, i.e., using a chemical method or chemical method combined with enzymatic method to synthesize target gene. The preparation of target gene fragment may also be commercially conducted by a contract research organization (CRO) in case that the sequence of target gene fragment is provided.

In some embodiments, the purification tag is a ST sequence tag (an amino acid sequence that helps the polypeptide of the present disclosure to form inclusion bodies) or a His tag.

In some embodiments, the expression host is a host cell. The host cell includes but is not limited to an individual cell, cell culture, or cell line. In some embodiments, the host cells include progeny of a single host cell. In some embodiments, a host cell can be transfected with a heterologous sequence including vectors encoding the polypeptide of the present disclosure. In some embodiments, said host cells may be prokaryotic cells, such as bacterial cells. In some embodiments, said host cells may be eukaryotic cells, such as yeast cells, animal cells, insect cells, plant cells and the like.

Examples of bacterial host cells that can be used to produce the polypeptide of the present disclosure include microorganisms belonging to the genus Escherichia, Serratia, Bacillus, Brevibacterium, Corynebacterium, Microbacterium, Pseudomonas and the like. For example, bacterial host cells may include, but not be limited to, Escherichia coli XL1-Blue, XL2-Blue, DH1, MC1000, KY3276, W1485, JM109, HB101. No. 49, i W3110, NY49, G1698, BL21, or TB1. Other bacterial host cells may include, but not be limited to, Serratia ficaria, Serratia fonticola, Serratia liquefaciens, Serratia marcescens, Bacillus subtilis, Bacillus amyloliquefaciens, Brevibacterium ammoniagenes, Brevibacterium immariophilum ATCC 14068. Brevibacterium saccharolyticum ATCC 14066, Brevibacterium flavum ATCC 14067, Brevibacterium lactofermentum ATCC 13869, Corynebacterium glutamicum ATCC 13032, Corynebacterium glutamicum ATCC 13869, Corynebacterium acetoacidophilum ATCC 13870, Microbacterium ammoniaphilum ATCC 15354, Pseudomonas putida, Pseudomonas sp, D-0110 and the like.

Examples of yeast cells that can be used to produce polypeptide of the present disclosure include microorganisms belonging to the genus Kluyveromyces, Trichosporon, Saccharomyces, Schizosaccharomyces, Schwanniomyces, Pichia, Candida and the like, such as Saccharomyces cerevisiae, Schizosaccharomyces pombe, Kluyveromyces lactis, Trichosporon pullulans, Schwanniomyces alluvius, Candida utilis and the like.

Examples of animal cells that can be used to produce the polypeptide of the present disclosure include mammalian cells, for example, Chinese hamster ovary cells (CHO) or monkey cells, such as COS cells, HepG2 cells, A549 cells, and any other cells available through ATCC or other depositories.

In some embodiments, the expression host is an Escherichia Coli host cell. In some embodiments, the formula of the fermentation medium used in the fermentation process is: yeast extract powder 10-50 g/L, peptone 10-30 g/L, ammonium sulfate 2-10 g/L, sodium chloride 2-10 g/L, potassium dihydrogen phosphate 0-10 g/L, dipotassium hydrogen phosphate 2-15 g/L, defoamer 0.01-0.1% (v/v), FeSO₄·7H₂O 0-0.1 g/L, ZnSO₄·7H₂O 0-0.02 g/L, CuSO₄·5H₂O 0-0.1 g/L, MnSO₄·5H₂O 0-0.05 g/L, CaCl₂·7H₂O 0-0.01 g/L, CoCl₂·6H₂O 0-0.01 g/L, Na₂MoO₄·2H₂O 0-0.01 g/L, H₃BO₃ 0-0.0005 g/L, and Biotin 0-0.005 g/L. In some embodiments, the fermentation process is performed at 37° C.

In some embodiments, the induced expression is realized by addition of isopropyl-beta-D-thiogalactopyranoside (IPTG) during the fermentation process. In some embodiments, the IPTG is added 0.5, 1, 2, 3, 4 or 5 hours after the initiation of the fermentation process. In some embodiments, the IPTG is added at a final concentration of 0.25, 0.5, 1, 2 or 4 mM. In some embodiments, the induced expression is performed at 37° C.

The crude purification of the present disclosure is a process during which the culture produced by the fermentation process is preliminary treated. In some embodiments, the crude purification comprises the steps of: collecting the cells and isolating the inclusion body proteins and/or cytoplasmic proteins after lysing the cells; subjecting the inclusion body proteins and/or cytoplasmic proteins to denaturation, renaturation and enzymatic digestion to obtain a crude product containing the crude polypeptide. In some embodiments, the crude purification comprises the steps of: collecting the culture medium, removing the cells and impurities, and obtaining the supernatant, i.e., a crude product containing the crude polypeptide.

The refined purification of the present disclosure is a process during which the crude product containing the crude polypeptide is purified with a chromatographic method. Examples of chromatographic methods that can be used to purify the polypeptide of the present disclosure include: ion exchange chromatography with a strong anion exchange resin, a weak anion exchange resin or a multimodal anion exchange resin; affinity chromatography; reversed phase chromatography with reversed phase packing materials; molecular sieve chromatography with size exclusion packing materials; and hydrophobic chromatography with hydrophobic packing materials.

More detailed information for preparing the polypeptide of the present disclosure may be found throughout of the disclosure of CN109913483A, e.g., Examples 1-4.

### Formulation

In another aspect, provided herein is a formulation comprises the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof and a pharmaceutically acceptable excipient. In some embodiments of the formulation, the polypeptide has at least 80% identity with any one selected from SEQ ID NO. 28, SEQ ID NOs. 57 and 62-74 or a fragment or variant thereof. In some embodiments of the formulation, the polypeptide has at least 85% identity with any one selected from SEQ ID NO. 28, SEQ ID NOs. 57 and 62-74 or a fragment or variant thereof. In some embodiments of the formulation, the polypeptide has at least 90% identity with any one selected from SEQ ID NO, 28, SEQ ID NOs. 57 and 62-74 or a fragment or variant thereof. In some embodiments of the formulation, the polypeptide has at least 95% identity with any one selected from SEQ ID NO. 28, SEQ ID NOs. 57 and 62-74 or a fragment or variant thereof. In some embodiments of the formulation, the polypeptide has at least 96%, at least 97%, at least 98%, at least 99% identity with any one selected from SEQ ID NO. 28, SEQ ID NOs, 57 and 62-74 or a fragment or variant thereof. In some embodiments of the formulation, the polypeptide comprises or is a sequence of any one selected from SEQ ID NO. 28, SEQ ID NOs. 57 and 62-74 or a fragment or variant thereof.

In some embodiments of the formulation, the pharmaceutically acceptable excipient includes but are not limited to inert solid diluents and fillers, diluents, sterile aqueous solution and various organic solvents, permeation enhancers, solubilizers and adjuvants.

In some embodiment of the formulation, the formulation may, for example, be in a form suitable for oral administration as a tablet, capsule, pill, powder, sustained release formulations, solution, suspension, for parenteral injection as a sterile solution, suspension or emulsion, for topical administration as a spray, ointment or cream. The formulation may be in unit dosage forms suitable for single administration. In some embodiment, the pharmaceutical composition may include other medicinal or pharmaceutical agents, carriers, adjuvants, etc.

In some embodiments, the formulation can be formulated as aqueous or oil suspensions, or emulsions, with sesame oil, corn oil, cottonseed oil, or peanut oil, as well as elixirs, mannitol, dextrose, or a sterile aqueous solution, and similar pharmaceutical vehicles.

In some embodiments, the formulation can be prepared as discrete dosage forms, such as capsules, cachets, or tablets, or liquids or aerosol sprays each containing a predetermined amount of an active ingredient as a powder or in granules, a solution, or a suspension in an aqueous or non-aqueous liquid, an oil-in-water emulsion, or a water-in-oil liquid emulsion.

In some embodiments, binders suitable for use in the formulation include, but are not limited to, corn starch, potato starch, or other starches, gelatin, natural and synthetic gums such as acacia, sodium alginate, alginic acid, other alginates, powdered tragacanth, guar gum, cellulose and its derivatives (e.g., ethyl cellulose, cellulose acetate, carboxymethyl cellulose calcium, sodium carboxymethyl cellulose), polyvinyl pyrrolidone, methyl cellulose, pre-gelatinized starch, hydroxypropyl methyl cellulose, microcrystalline cellulose, and mixtures thereof.

In some embodiments, fillers suitable for use in the formulation include, but are not limited to, talc, calcium carbonate (e.g., granules or powder), microcrystalline cellulose, powdered cellulose, dextrates, kaolin, mannitol, silicic acid, sorbitol, starch, pre-gelatinized starch, and mixtures thereof.

In some embodiments, disintegrants that can be used in the formulation include, but are not limited to, agar-agar, alginic acid, calcium carbonate, microcrystalline cellulose, croscarmellose sodium, crospovidone, polacrilin potassium, sodium starch glycolate, potato or tapioca starch, other starches, pre-gelatinized starch, other starches, clays, other algins, other celluloses, gums or mixtures thereof.

In some embodiments, lubricants which can be used in the formulation include, but are not limited to, calcium stearate, magnesium stearate, mineral oil, light mineral oil, glycerin, sorbitol, mannitol, polyethylene glycol, other glycols, stearic acid, sodium lauryl sulfate, talc, hydrogenated vegetable oil (e.g., peanut oil, cottonseed oil, sunflower oil, sesame oil, olive oil, corn oil, and soybean oil), zinc stearate, ethyl oleate, ethyl laureate, agar, or mixtures thereof. Additional lubricants include, for example, a syloid silica gel, a coagulated aerosol of synthetic silica, or mixtures thereof. A lubricant can optionally be added, in an amount of less than about 1 weight percent of the formulation.

In some embodiments, surfactants which can be used in the formulation include, but are not limited to, hydrophilic surfactants, lipophilic surfactants, and mixtures thereof. That is, a mixture of hydrophilic surfactants may be employed, a mixture of lipophilic surfactants may be employed, or a mixture of at least one hydrophilic surfactant and at least one lipophilic surfactant may be employed. Surfactants with lower HLB values are more lipophilic or hydrophobic, and have greater solubility in oils, while surfactants with higher HLB values are more hydrophilic, and have greater solubility in aqueous solutions. Hydrophilic surfactants are generally considered to be those compounds having an HLB value greater than about 10, as well as anionic, cationic, or zwitterionic compounds for which the HLB scale is not generally applicable. Similarly, lipophilic (i.e., hydrophobic) surfactants are compounds having an HLB value equal to or less than about 10. However, HLB value of a surfactant is merely a rough guide generally used to enable formulation of industrial, pharmaceutical and cosmetic emulsions.

Hydrophilic surfactants may be either ionic or non-ionic. Suitable ionic surfactants include, but are not limited to, alkylammonium salts; fusidic acid salts; fatty acid derivatives of amino acids, oligopeptides, and polypeptides; glyceride derivatives of amino acids, oligopeptides, and polypeptides; lecithins and hydrogenated lecithins; lysolecithins and hydrogenated lysolecithins; phospholipids and derivatives thereof; lysophospholipids and derivatives thereof; carnitine fatty acid ester salts; salts of alkylsulfates; fatty acid salts; sodium docusate; acyl lactylates; mono- and di-acetylated tartaric acid esters of mono- and di-glycerides; succinylated mono- and di-glycerides; citric acid esters of mono- and di-glycerides; and mixtures thereof.

Within the aforementioned group, ionic surfactants include, by way of example: lecithins, lysolecithin, phospholipids, lysophospholipids and derivatives thereof; carnitine fatty acid ester salts; salts of alkylsulfates; fatty acid salts; sodium docusate; acylactylates; mono- and di-acetylated tartaric acid esters of mono- and di-glycerides; succinylated mono- and di-glycerides; citric acid esters of mono- and di-glycerides; and mixtures thereof.

Ionic surfactants may be the ionized forms of lecithin, lysolecithin, phosphatidylcholine, phosphatidylethanolamine, phosphatidylglycerol, phosphatidic acid, phosphatidylserine, lysophosphatidylcholine, lysophosphatidylethanolamine, lysophosphatidylglycerol, lysophosphatidic acid, lysophosphatidylserine, PEG-phosphatidylethanolamine, PVP-phosphatidylethanolamine, lactylic esters of fatty acids, stearoyl-2-lactylate, stearoyl lactylate, succinylated monoglycerides, mono/diacetylated tartaric acid esters of mono/diglycerides, citric acid esters of mono/diglycerides, cholylsarcosine, caproate, caprylate, caprate, laurate, myristate, palmitate, oleate, ricinoleate, linoleate, linolenate, stearate, lauryl sulfate, teracecyl sulfate, docusate, lauroyl carnitines, palmitoyl carnitines, myristoyl carnitines, and salts and mixtures thereof.

Hydrophilic non-ionic surfactants may include, but are not limited to, alkylglucosides; alkylmaltosides; alkylthioglucosides; lauryl macrogolglycerides; polyoxyalkylene alkyl ethers such as polyethylene glycol alkyl ethers; polyoxyalkylene alkylphenols such as polyethylene glycol alkyl phenols; polyoxyalkylene alkyl phenol fatty acid esters such as polyethylene glycol fatty acids monoesters and polyethylene glycol fatty acids diesters; polyethylene glycol glycerol fatty acid esters; polyglycerol fatty acid esters; polyoxyalkylene sorbitan fatty acid esters such as polyethylene glycol sorbitan fatty acid esters; hydrophilic transesterification products of a polyol with at least one member of the group consisting of glycerides, vegetable oils, hydrogenated vegetable oils, fatty acids, and sterols; polyoxyethylene sterols, derivatives, and analogues thereof: polyoxyethylated vitamins and derivatives thereof; polyoxyethylene-polyoxypropylene block copolymers; and mixtures thereof; polyethylene glycol sorbitan fatty acid esters and hydrophilic transesterification products of a polyol with at least one member of the group consisting of triglycerides, vegetable oils, and hydrogenated vegetable oils. The polyol may be glycerol, ethylene glycol, polyethylene glycol, sorbitol, propylene glycol, pentaerythritol, or a saccharide.

Other hydrophilic-non-ionic surfactants include, without limitation, PEG-10 laurate, PEG-12 laurate, PEG-20 laurate, PEG-32 laurate, PEG-32 dilaurate, PEG-12 oleate, PEG-15 oleate, PEG-20 oleate, PEG-20 dioleate, PEG-32 oleate, PEG-200 oleate, PEG-400 oleate, PEG-15 stearate, PEG-32 distearate, PEG-40 stearate, PEG-100 stearate, PEG-20 dilaurate, PEG-25 glyceryl trioleate, PEG-32 dioleate, PEG-20 glyceryl laurate, PEG-30 glyceryl laurate, PEG-20 glyceryl stearate, PEG-20 glyceryl oleate, PEG-30 glyceryl oleate, PEG-30 glyceryl laurate, PEG-40 glyceryl laurate, PEG-40 palm kernel oil, PEG-50 hydrogenated castor oil, PEG-40 castor oil, PEG-35 castor oil, PEG-60 castor oil, PEG-40 hydrogenated castor oil, PEG-60 hydrogenated castor oil, PEG-60 corn oil, PEG-6 caprate/caprylate glycerides, PEG-8 caprate/caprylate glycerides, polyglyceryl-10 laurate, PEG-30 cholesterol, PEG-25 phyto sterol, PEG-30 soya sterol, PEG-20 trioleate, PEG-40 sorbitan oleate, PEG-80 sorbitan laurate, polysorbate 20, polysorbate 80, POE-9 lauryl ether, POE-23 lauryl ether, POE-10 oleyl ether, POE-20 oleyl ether, POE-20 stearyl ether, tocopheryl PEG-100 succinate, PEG-24 cholesterol, polyglyceryl-10oleate, Tween 40, Tween 60, sucrose monostearate, sucrose monolaurate, sucrose monopalmitate, PEG 10-100 nonyl phenol series, PEG 15-100 octyl phenol series, and poloxamers.

Suitable lipophilic surfactants include, by way of example only: fatty alcohols; glycerol fatty acid esters; acetylated glycerol fatty acid esters; lower alcohol fatty acids esters; propylene glycol fatty acid esters; sorbitan fatty acid esters; polyethylene glycol sorbitan fatty acid esters; sterols and sterol derivatives; polyoxyethylated sterols and sterol derivatives; polyethylene glycol alkyl ethers; sugar esters; sugar ethers; lactic acid derivatives of mono- and di-glycerides; hydrophobic transesterification products of a polyol with at least one member of the group consisting of glycerides, vegetable oils, hydrogenated vegetable oils, fatty acids and sterols; oil-soluble vitamins/vitamin derivatives; and mixtures thereof. Within this group, preferred lipophilic surfactants include glycerol fatty acid esters, propylene glycol fatty acid esters, and mixtures thereof, or are hydrophobic transesterification products of a polyol with at least one member of the group consisting of vegetable oils, hydrogenated vegetable oils, and triglycerides.

In one embodiment, the formulation may include a solubilizer to ensure good solubilization and/or dissolution of the artificial polypeptide or mutant of the present invention and to minimize precipitation of the artificial polypeptide or mutant of the present invention. This can be especially important for compositions for non-oral use, e.g., compositions for injection. Examples of suitable solubilizers include, but are not limited to, the following: alcohols and polyols, such as ethanol, isopropanol, butanol, benzyl alcohol, ethylene glycol, propylene glycol, butanediols and isomers thereof, glycerol, pentaerythritol, sorbitol, mannitol, transcutol, dimethyl isosorbide, polyethylene glycol, polypropylene glycol, polyvinylalcohol, hydroxypropyl methylcellulose and other cellulose derivatives, cyclodextrins and cyclodextrin derivatives; ethers of polyethylene glycols having an average molecular weight of about 200 to about 6000, such as tetrahydrofurfuryl alcohol PEG ether (glycofurol) or methoxy PEG; amides and other nitrogen-containing compounds such as 2-pyrrolidone, 2-piperidone, ε-caprolactam, N-alkylpyrrolidone, N- and hydroxyalkylpyrrolidone, N-alkylpiperidone, N-alkylcaprolactam, dimethylacetamide polyvinylpyrrolidone; esters such as ethyl propionate, tributylcitrate, acetyl triethylcitrate, acetyl tributyl citrate, triethylcitrate, ethyl oleate, ethyl caprylate, ethyl butyrate, triacetin, propylene glycol monoacetate, propylene glycol diacetate, ε-caprolactone and isomers thereof, δ-valerolactone and isomers thereof, β-butyrolactone and isomers thereof; and other solubilizers known in the art, such as dimethyl acetamide, dimethyl isosorbide, N-methyl pyrrolidones, monooctanoin, diethylene glycol monoethyl ether, and water.

Mixtures of solubilizers may also be used. Examples include, but not limited to, triacetin, triethylcitrate, ethyl oleate, ethyl caprylate, dimethylacetamide, N-methylpyrrolidone, N-hydroxyethylpyrrolidone, polyvinylpyrrolidone, hydroxypropyl methylcellulose, hydroxypropyl cyclodextrins, ethanol, polyethylene glycol 200-100, glycofurol, transcutol, propylene glycol, and dimethyl isosorbide. Particularly preferred solubilizers include sorbitol, glycerol, triacetin, ethyl alcohol, PEG-400, glycofurol and propylene glycol.

In addition, an acid or a base may be incorporated into the formulation to facilitate processing, to enhance stability, or for other reasons. Examples of pharmaceutically acceptable bases include amino acids, amino acid esters, ammonium hydroxide, potassium hydroxide, sodium hydroxide, sodium hydrogen carbonate, aluminum hydroxide, calcium carbonate, magnesium hydroxide, magnesium aluminum silicate, synthetic aluminum silicate, synthetic hydrocalcite, magnesium aluminum hydroxide, diisopropylethylamine, ethanolamine, ethylenediamine, triethanolamine, triethylamine, triisopropanolamine, trimethylamine, tris(hydroxymethyl)aminomethane (TRIS) and the like. Also suitable are bases that are salts of a pharmaceutically acceptable acid, such as acetic acid, acrylic acid, adipic acid, alginic acid, alkanesulfonic acid, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid, and the like. Salts of polyprotic acids, such as sodium phosphate, disodium hydrogen phosphate, and sodium dihydrogen phosphate can also be used. When the base is a salt, the cation can be any convenient and pharmaceutically acceptable cation, such as ammonium, alkali metals, alkaline earth metals, and the like. Example may include, but not limited to, sodium, potassium, lithium, magnesium, calcium and ammonium.

Suitable acids are pharmaceutically acceptable organic or inorganic acids. Examples of suitable inorganic acids include hydrochloric acid, hydrobromic acid, hydriodic acid, sulfuric acid, nitric acid, boric acid, phosphoric acid, and the like. Examples of suitable organic acids include acetic acid, acrylic acid, adipic acid, alginic acid, alkanesulfonic acids, amino acids, ascorbic acid, benzoic acid, boric acid, butyric acid, carbonic acid, citric acid, fatty acids, formic acid, fumaric acid, gluconic acid, hydroquinosulfonic acid, isoascorbic acid, lactic acid, maleic acid, methanesulfonic acid, oxalic acid, para-bromophenylsulfonic acid, propionic acid, p-toluenesulfonic acid, salicylic acid, stearic acid, succinic acid, tannic acid, tartaric acid, thioglycolic acid, toluenesulfonic acid, uric acid and the like,

### Ophthalmic Formulation

Dry eye (DE) is a multifactorial disorder of tears and ocular surface, which results in symptoms such as eye discomfort, visual disturbance, tear film instability, and ocular surface damage.

Ocular surface system comprises the cornea, conjunctiva, lacrimal glands, meibomian glands, nasolacrimal duct, and their associated connective tissue matrices, as well as the eyelids and eyelashes. In DE, the ocular surface epithelium may undergo squamous metaplasia, manifested by loss of goblet cells, mucin deficiency and keratinization, further resulting in tear film instability, “Cornea fluorescein staining” which detects ocular surface damage has been used to assess DE. The fluorescein does not stain intact corneal epithelium but corneal stroma, thus can be used to identify the area of the epithelial loss. In the test, fluorescein dye is instilled into an eye as a liquid drop or via a paper strip. Then the damage is evaluated by fluorescein staining score ranging from 0 to 15, where a score 25 is considered as DE.

Tear film is a three-layered structure comprising a mucoidal basal layer, an aqueous component and a superficial lipid layer. Tear film is formed and maintained by blinking, and the composition of tear film can be affected by systemic or ocular conditions. Tear film stability can be evaluated by various assessments, such as “tear breakup time” or “TBUT,” TBUT is a clinical test which measures the interval between the individual's last complete blink and the breakup of the tear film. The test can be used to assess DE or DE associated disorder. To measure TBUT, fluorescein is instilled into a subject's tear film and then the subject is required not to blink while the tear film is observed under a broad beam of cobalt blue illumination, TBUT is recorded as the number of seconds that elapse between the last blink and the appearance of the first dry spot in the tear film, where TBUT of less than 10 seconds is considered as abnormal tear film and less than 5 seconds is indicative of DE.

The “DE or DE associated disorder” as used herein refers to any form of DE and related symptoms, including but not limited to dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia, Stevens-Johnson syndrome, ocular pemphigoid blepharitis marginal, eyelid-closure failure, corneal ulcer, blepharitis and eye redness, and the like.

In another aspect, the present disclosure provides an ophthalmic formulation for treating or preventing dry eye (DE) or DE associated disorders, and the ophthalmic formulation comprises the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof.

In another aspect, the present disclosure provides an ophthalmic formulation for treating or preventing dry eye (DE) or DE associated disorders, and the ophthalmic formulation comprises a polypeptide having at least 70% identity with any one selected from SEQ ID NO. 28, SEQ ID NOs. 57 and 62-74 or a fragment or variant thereof. In some embodiments of the ophthalmic formulation, the polypeptide comprises a sequence having at least 80% identity with any one selected from SEQ ID NO. 28, SEQ ID NOs. 57 and 62-74 or a fragment or variant thereof. In some embodiments of the ophthalmic formulation, the polypeptide comprises a sequence having at least 85% identity with any one selected from SEQ ID NO. 28, SEQ ID NOs. 57 and 62-74 or a fragment or variant thereof. In some embodiments of the ophthalmic formulation, the polypeptide comprises a sequence having at least 90% identity with any one selected from SEQ ID NO. 28, SEQ ID NOs. 57 and 62-74 or a fragment or variant thereof. In some embodiments of the ophthalmic formulation, the polypeptide comprises a sequence having at least 95% identity with any one selected from SEQ ID NO. 28, SEQ ID NOs. 57 and 62-74 or a fragment or variant thereof. In some embodiments of the ophthalmic formulation, the polypeptide comprises a sequence having at least 96%, at least 97%, at least 98%, at least 99% identity with any one selected from SEQ ID NO. 28, SEQ ID NOs. 57 and 62-74 or a fragment or variant thereof. In some embodiments of the ophthalmic formulation, the polypeptide comprises a sequence of any one selected from SEQ ID NO. 28, SEQ ID NOs. 57 and 62-74 or a fragment or variant thereof.

In some embodiments, the ophthalmic formulation of the present disclosure is formulated as a solution. In some embodiments, the ophthalmic formulation of the present disclosure is formulated as an eye drop solution. In some embodiments, the ophthalmic formulation of the present disclosure is formulated as a gel. In some embodiments, the ophthalmic formulation of the present disclosure is formulated as an ointment. In some embodiments, the ophthalmic formulation of the present disclosure is formulated as a suspension, a semi-liquid, a semi-solid gel, a cream, a foam gel, a contact lens solution, an eyewash, and the like.

In some embodiments, the ophthalmic formulation is prepared by dissolving the polypeptide of the present disclosure in an aqueous solution. Aqueous solutions and diluents that can be used in preparing the ophthalmic formulation include but are not limited to distilled water, physiological saline, and the like.

In some embodiments, the ophthalmic formulation is prepared by dissolving the polypeptide of the present disclosure in a non-aqueous solution or diluents. Non-aqueous solutions and diluents include but are not limited to edible (e.g. vegetable) oil, liquid paraffin, mineral oil, propylene glycol, p-octyldodecanol, polysorbate, macrogols, aluminum monostearate and the like.

In some embodiments, the ophthalmic formulation can be formulated by admixing, diluting or dissolving the polypeptide of the present disclosure, with appropriate pharmaceutical excipients, such as disintegrators, binders, lubricants, diluents, buffers, antiseptics, moistening agents, emulsifiers, dispersing agents, stabilizing agents and dissolving aids in accordance with conventional methods, and in a conventional manner depending upon the dosage form.

In some embodiments, buffering agents are added to keep the pH constant and can include pharmaceutically acceptable buffering agents such as borate buffer, citrate buffer, tartrate buffer, phosphate buffer, acetate buffer and a Tris-HCl buffer (comprising tris(hydroxymethyl) aminomethane and HCl). For example, a Tris-HCl buffer having pH of 7.4 comprises 3 g/l of tris-(hydroxymethyl)-aminomethane and 0.76 g/l of HCl. In some embodiments, the buffer is 10× phosphate buffer saline (“PBS”) or 5×PBS solution. Buffering agents are added to the ophthalmic formulation in an amount that provides sufficient buffer capacity for the expected physiological conditions.

Other buffers can be used in the ophthalmic formulation of the present disclosure include, but are not limited to, buffers based on HEPES (N-{2-hydroxyethyl}piperazine-N′-{2-ethanesulfonic acid}) having pKa of 7.5 at 25° C. and pH in the range of about 6.8-8.2; BES (N,N-bis {2-hydroxyethyl}2-aminoethanesulfonic acid) having pKa of 7.1 at 25° C. and pH in the range of about 6.4-7.8; MOPS (3-{N-morpholino}propanesulfonic acid) having pKa of of 7.2 at 25° C. and pH in the range of about 6.5-7.9; TES (N-tris{hydroxymethyl}-methyl-2-aminoethanesulfonic acid) having pKa of 7.4 at 25° C. and pH in the range of about 6.8-8.2; MOBS (4-{N-morpholino}butanesulfonic acid) having pKa of 7.6 at 25° C. and pH in the range of about 6.9-8.3; DIPSO (3-(N,N-bis{2-hydroxyethyl}amino)-2-hydroxypropane)) having pKa of 7.52 at 25° C. and pH in the range of about 7-8.2; TAPS ({(2-hydroxy-3 {tris(hydroxymethyl)methylamino}-1-propanesulfonic acid)) having pKa of 7.61 at 25° C. and pH in the range of about 7-8.2; TAPS ({(2-hydroxy-1,1-bis(hydroxymethyl)ethyl)aminol-l-propanesulfonic acid)) having pKa of 8.4 at 25° C. and pH in the range of about 7.7-9.1; TABS (N-tris(hydroxymethyl)methyl-4-aminobutanesulfonic acid) having pKa of 8.9 at 25° C. and pH in the range of about 8.2-9.6; AMPSO (N-(1,1-dimethyl-2-hydroxyethyl)-3-amino-2-hydroxypropanesulfonic acid)) having pKa of 9.0 at 25° C. and pH in the range of about 8.3-9.7; CHES (2-cyclohexylamino) ethanesulfonic acid) having pKa of 9.5 at 25° C. and pH in the range of about 8.6-10.0; CAPSO (3-(cyclohexylamino)-2-hydroxy-1-propanesulfonic acid) having pKa of 9.6 at 25° C. and pH in the range of about 8.9-10.3; and CAPS (3-(cyclohexylamino)-1-propane sulfonic acid) having pKa of 10.4 at 25° C. and pH in the range of about 9.7-11.1.

In some embodiments, isotonizers can be added to make the formulation isotonic with the tear. Isotonizers include, but are not limited to, sugars such as dextrose, glucose, sucrose and fructose; sugar alcohols such as mannitol and sorbitol; polyhydric alcohols such as glycerol, polyethylene glycol and propylene glycol; and salts such as sodium chloride, sodium citrate, benzalkonium chloride, phedrine chloride, potassium chloride, procaine chloride, chloram phenicol, and sodium succinate. Isotonizers are added in an amount that makes the osmotic pressure of the ophthalmic formulation equal to that of the tear.

In some embodiments, the ophthalmic formulation comprises a tonicity agent. Tonicity agents suitable for the ophthalmic formulation of the present disclosure include but are not limited to sodium chloride, sodium nitrate, sodium sulfate, sodium bisulfate, potassium chloride, calcium chloride, magnesium chloride, zinc chloride, potassium acetate, sodium acetate, sodium bicarbonate, sodium carbonate, sodium thiosulfate, magnesium sulfate, disodium hydrogen phosphate, sodium dihydrogen phosphate, potassium dihydrogen phosphate, dextrose, mannitol, sorbitol, dextrose, sucrose, urea, propylene glycol, glycerin, and a combination thereof.

In some embodiments, the ophthalmic formulation comprises a viscosity-enhancing agent. Viscosity-enhancing agents suitable for the ophthalmic formulation of the present disclosure include but are not limited to monomeric polyols such as tyloxapol, glycerol, propylene glycol, ethylene glycol; polymeric polyols such as polyethylene glycol (e.g., PEG 300, PEG 400); cellulose-based polymer such as cellulose gum, alkylcellulose, hydroxyl-alkyl cellulose, hydroxyl-alkyl alkylcellulose, carboxy-alkyl cellulose, hydroxyethylcellulose hypromellose, hydroxypropylmethyl cellulose, methylcellulose, carboxymethylcellulose sodium, hydroxylpropylcellulose; dextrans such as dextran 70; water-soluble proteins such as gelatin; vinyl polymers such as polyvinyl alcohol, polyvinyl pyrollidine; other polyols such as polysorbate 80, povidone; polysaccharides and glycosaminoglycans such as hyaluronan, chondroitin sulfate; and combinations thereof.

In some embodiments, preservatives can be added to maintain the integrity of the ophthalmic formulation. Preservatives include, but are not limited to, sorbic acid, benzalkonium chloride, benzododecinium bromide, parabens, chlorobutanol, benzylic alcohol, phenylethyl alcohol, edentate disodium, sorbic acid, polyquaternium-1, or other agents known to those skilled in the art.

In addition to the above, in some embodiments, it is desirable to use additional agents which include, but are not limited to, stabilizers suitable for the ophthalmic formulation of the present disclosure such as sodium sulfite, sodium carbonate, and propylene glycol; antioxidants such as ascorbic acid, sodium ascorbate, butylated hydroxy toluene (BHT), butylated hydroxyanisole (BHA), tocopherol, sodium thiosulfate; and/or chelating agents such as ethylene-diamine-tetra-acetic acid (EDTA), ethylene glycol-bis-(2-aminoethyl)-N,N,N,N-tetraacetic acid (EGTA) and sodium citrate.

Eye drops, ophthalmic gels and/or ophthalmic ointments can be prepared by aseptic manipulation. Alternatively, sterilization of the composition can be performed at a suitable stage of preparation. In some embodiments, the sterile composition can be prepared by mixing sterile ingredients aseptically. In some embodiments, the sterile composition can be prepared by first mixing the ingredients then sterilizing the final preparation. Sterilization methods can include, but are not limited to, heat sterilization, irradiation and filtration.

In some embodiments, ophthalmic ointments (eye ointments) can be aseptically prepared by mixing the polypeptide of the present disclosure into a base that is used for preparation of eye ointments followed by formulation into pharmaceutical preparations with any method known in the art. Typical bases for eye ointments are exemplified by vaseline, jelene 50, plastibase and macrogol. In addition, surfactants may be added to increase hydrophilia.

In some embodiments, additives may be added to the ophthalmic formulation such as eye drops, ophthalmic gels and/or ophthalmic ointments as needed. In some embodiments, the additives include but are not limited to additional ingredients, additives, carrier suitable for use in contact on or around the eye without undue toxicity, incompatibility, instability, irritation, allergic response, and the like.

In some embodiments, the ophthalmic formulation of the present disclosure is formulated for topical administration. In some embodiments, the ophthalmic formulation of the present disclosure can be locally administered to the eye, such as subconjunctivally, retrobulbarly, periocularly, subretinally, suprachoroidally, or intraocularly administered to the eye.

In some embodiments, the ophthalmic formulation can be delivered to the ocular surface, interconnecting innervation, conjuncitva, lacrimal glands, or meibomian glands. It is envisioned that effective treatment can encompass administering therapeutic agents of the present invention via oral administration, topical administration, via injection, intranasally, rectally, transdermally, via an impregnated or coated device such as an ocular insert or implant, or iontophoretically, amongst other routes of administration.

In some embodiments, the ophthalmic formulation of the present disclosure is formulated for injection. For administration via injection, the ophthalmic formulation can be injected intramuscularly, intra-arterially, subcutaneously, or intravenously. A pump mechanism may be employed to administer the pharmaceutical composition over a preselected period. For some embodiments of the invention it is desirable to deliver drug locally, thus injections may be made periocularly, intraocularly, subconjunctively, retrobulbarly, or intercamerally.

In some embodiments, the ophthalmic formulation of the present invention may be administered to the ocular surface via a pump-catheter system, or released from within a continuous or selective release device such as, e.g., membranes such as, but not limited to, those employed in the Ocusert™ System (Alza Corp, Palo Alto, CA). The pharmaceutical compositions can be incorporated within, carried by or attached to contact lenses which are then worn by the subject. The pharmaceutical compositions can be sprayed onto ocular surface.

In some embodiments, the ophthalmic formulation of the present disclosure is formulated for systemic delivery. For systemic administration, the ophthalmic formulation can be formulated for and administered orally. For administration that may result in either regional or systemic distribution of the therapeutic agents, the formulation of the invention may be administered intranasally, transdermally, or via some forms of oral administration, e.g. with use of a mouthwash or lozenge incorporating a polypeptide of the present disclosure that is poorly absorbed from the G.I. For administration that may result in regional or local delivery of the formulation of the invention, iontophoretic or topical administration may be used.

In some embodiments, the ophthalmic formulation comprises from about 0.001 μM to about 100 μM of polypeptide of the present disclosure. In some embodiments, the ophthalmic formulation comprises from about 0.01 μM to about 20 μM of polypeptide of the present disclosure. In some embodiments, the ophthalmic formulation comprises from about 0.1 μM to about 5 μM of polypeptide of the present disclosure. In some embodiments, the ophthalmic formulation comprises from about 0.2 μM to about 3 μM of polypeptide of the present disclosure. In some embodiments, the ophthalmic formulation comprises from about 0.1 μM to about 10 μM of polypeptide of the present disclosure. In some embodiments, the ophthalmic formulation comprises from about 1 μM to about 5 μM of polypeptide of the present disclosure. In some embodiments, the ophthalmic formulation comprises from about 1 μM to about 10 μM of polypeptide of the present disclosure. In some embodiments, the ophthalmic formulation comprises from about 5 μM to about 10 μM of polypeptide of the present disclosure. In some embodiments, the ophthalmic formulation comprises from about 10 μM to about 50 μM of polypeptide of the present disclosure. In some embodiments, the ophthalmic formulation comprises from about 20 μM to about 50 μM of polypeptide of the present disclosure. In some embodiments, the ophthalmic formulation comprises from about 5 μM to about 50 μM of polypeptide of the present disclosure. In some embodiments, the ophthalmic formulation comprises from about 1 μM to about 50 μM of polypeptide of the present disclosure. In some embodiments, the ophthalmic formulation comprises from about 1 μM to about 20 M of polypeptide of the present disclosure. In some embodiments, the ophthalmic formulation comprises from about 5 μM to about 20 μM of polypeptide of the present disclosure. In some embodiments, the ophthalmic formulation comprises from about 10 μM to about 20 μM of polypeptide of the present disclosure. In some embodiments, the ophthalmic formulation comprises higher than about 0.001, 0.05, 0.1, 0.2, 0.3, 0.4, 0.5, 0.6, 0.7, 0.8, 0.9, 1, 1.1, 1.2, 1.3, 1.4, 1.5, 1.6, 1.7, 1.8, 1.9, 2.0, 2.1, 2.2, 2.3, 2.4, 2.5, 2.6, 2.7, 2.8, 2.9, or 3.0 μM of polypeptide of the present disclosure. In some embodiments, the ophthalmic formulation comprises lower than 100, 90, 80, 70, 60, 50, 40, 30, 20, 25, 20, 19, 18, 17, 16, 15, 14, 13, 12, 11, 10, 9, 8, 7, 6, 5, or 4 μM of polypeptide of the present disclosure.

In some embodiments, the ophthalmic formulation can be formulated into a dosage form from about 0.01 to about 10 ml for use once or multiple times. In some embodiments, the ophthalmic formulation is formulated into a unit dosage form to provide a total daily dosage of from about 0.01 to about 2 ml. In some embodiments, the ophthalmic formulation can be formulated into a unit dosage form to provide a total weekly dosage of from about 1 ml to about 5 ml. In some embodiments, the ophthalmic formulation can be formulated into a unit dosage form to provide a total monthly dosage of from about 1 ml to about 20 ml.

### Treatment of DE

Also provided herein is a method for treating or preventing DE or DE associated disorder, stimulating tears, stabilizing tear film or any combination thereof in a subject in need thereof, comprising administrating a therapeutically effective amount of the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof to a subject in need thereof. In some embodiments, the polypeptide has at least 70% identity with anyone selected from SEQ ID NO. 28, SEQ ID NOs. 57 and 62-74, or a fragment or variant thereof. In some embodiments, the polypeptide has at least 75% identity with anyone selected from SEQ ID NO. 28, SEQ ID NOs. 57 and 62-74, or a fragment or variant thereof. In some embodiments, the polypeptide has at least 80% identity with anyone selected from SEQ ID NO, 28, SEQ ID NOs. 57 and 62-74, or a fragment or variant thereof. In some embodiments, the polypeptide has at least 85% identity with anyone selected from SEQ ID NO. 28, SEQ ID NOs. 57 and 62-74, or a fragment or variant thereof. In some embodiments, the polypeptide has at least 90% identity with anyone selected from SEQ ID NO. 28, SEQ ID NOs. 57 and 62-74, or a fragment or variant thereof. In some embodiments, the polypeptide has at least 95% identity with anyone selected from SEQ ID NO. 28, SEQ ID NOs. 57 and 62-74, or a fragment or variant thereof. In some embodiments, the polypeptide has at least 96%, at least 97%, at least 98%, or at least 99% identity with anyone selected from SEQ ID NO. 28, SEQ ID NOs. 57 and 62-74, or a fragment or variant thereof. In some embodiments, the polypeptide comprises or is an amino acid sequence of anyone selected from SEQ ID NO. 28, SEQ ID NOs. 57 and 62-74, or a fragment or variant thereof.

In some embodiments, provided herein is a method for treating or preventing DE or DE associated disorder, stimulating tears, stabilizing tear film or any combination thereof in a subject in need thereof, comprising administrating a therapeutically effective amount of the ophthalmic formulation of the present disclosure to a subject in need thereof.

In some embodiments, said DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia, Stevens-Johnson syndrome, ocular pemphigoid blepharitis marginal, eyelid-closure failure, corneal ulcer, blepharitis and eye redness, and the like.

Age is another factor which can affect tear composition and/or tear film stability. In some embodiments, the subject of the present disclosure has an age of older than 40. In some embodiments, the subject of the present disclosure has an age of older than 50. In some embodiments, the subject of the present disclosure has an age of older than 60. In some embodiments, the subject of the present disclosure has an age of older than 70, older than 75, older than 80, older than 85, or older than 90.

In some embodiments, the treatment of the present disclosure can be used to treat or prevent tear-deficient DE, in which lacrimal gland fails to produce enough of the watery component of tears to maintain a healthy eye surface. In some embodiments, the treatment of the present disclosure can be used to stimulate tear secretion, increase tear volume, regulate tear composition, improve tear clearance and/or osmolarity, or any combination thereof. In some embodiments, the method of the present disclosure can be used to improve tear film stability or tear film composition. In some embodiments, the treatment of the present disclosure can be used to improve ocular surface damage.

In some embodiments, the treatment of the present disclosure results in partial or total elimination of DE or DE-associated disorders, stimulation of tears, stabilization of tear film or any combination thereof of the subject. In some embodiment, one or more signs or symptoms of DE or DE-associated disorders are reduced in severity or duration by about 2%, about 5%, about 10%, about 15%, about 20%, about 25%, about 30%, about 35%, about 40%, about 45%, about 50%, about 55%, about 60%, about 65%, about 70%, about 75%, about 80%, about 85%, about 90%, about 95%, or about 100% following administration of the therapeutically effective amount of the polypeptide of the present disclosure to the subject. In some embodiments, one or more signs or symptoms of DE or DE-associated disorders are reduced in severity or duration by about 2-fold, about 5-fold, about 10-fold, about 15-fold, about 20-fold, about 25-fold, about 30-fold, about 35-fold, about 40-fold, about 45-fold, about 50-fold, about 55-fold, about 60-fold, about 65-fold, about 70-fold, about 75-fold, about 80-fold, about 85-fold, about 90-fold, about 95-fold, about 100-fold, or more, following administration of the therapeutically effective amount of the polypeptide of the present disclosure to the subject.

In some embodiments, one eye or both eyes of the subject of the present disclosure have TBUT equal or less than 10s prior to the treatment. In some embodiments, one eye or both eyes of the subject of the present disclosure have TBUT equal or less than 9s prior to the treatment. In some embodiments, one eye or both eyes of the subject of the present disclosure have TBUT equal or less than 8s prior to the treatment. In some embodiments, one eye or both eyes of the subject of the present disclosure have TBUT equal or less than 7s prior to the treatment. In some embodiments, one eye or both eyes of the subject of the present disclosure have TBUT equal or less than 6s prior to the treatment. In some embodiments, one eye or both eyes of the subject of the present disclosure have TBUT equal or less than 5s prior to the treatment. In some embodiments, one eye or both eyes of the subject of the present disclosure have TBUT equal or less than 4s prior to the treatment. In some embodiments, one eye or both eyes of the subject of the present disclosure have TBUT equal or less than 3s prior to the treatment. In some embodiments, one eye or both eyes of the subject of the present disclosure have TBUT equal or less than 2s prior to the treatment. In some embodiments, one eye or both eyes of the subject of the present disclosure have TBUT equal or less than Is prior to the treatment.

In some embodiments, TBUT of one eye of the subject is increased following administration of the therapeutically effective amount of the polypeptide of the present disclosure to the subject. In some embodiments, TBUT of both eyes of the subject is increased following administration of the therapeutically effective amount of the polypeptide of the present disclosure to the subject. In some embodiments, TBUT of one eye or both eyes of the subject are increased by at least 0.5s following administration of the therapeutically effective amount of the polypeptide of the present disclosure to the subject. In some embodiments, TBUT of one eye or both eyes of the subject are increased by at least Is following administration of the therapeutically effective amount of the polypeptide of the present disclosure to the subject. In some embodiments, TBUT of one eye or both eyes of the subject are increased by at least 2s following administration of the therapeutically effective amount of the polypeptide of the present disclosure to the subject. In some embodiments, TBUT of one eye or both eyes of the subject are increased by at least 3s following administration of the therapeutically effective amount of the polypeptide of the present disclosure to the subject. In some embodiments, TBUT of one eye or both eyes of the subject are increased by at least 4s following administration of the therapeutically effective amount of the polypeptide of the present disclosure to the subject. In some embodiments, TBUT of one eye or both eyes of the subject are increased by at least 5s following administration of the therapeutically effective amount of the polypeptide of the present disclosure to the subject. In some embodiments, TBUT of one eye or both eyes of the subject are increased by at least 6s following administration of the therapeutically effective amount of the polypeptide of the present disclosure to the subject. In some embodiments, TBUT of one eye or both eyes of the subject are increased by at least 7s following administration of the therapeutically effective amount of the polypeptide of the present disclosure to the subject. In some embodiments, TBUT of one eye or both eyes of the subject are increased by at least 8s following administration of the therapeutically effective amount of the polypeptide of the present disclosure to the subject. In some embodiments, TBUT of one eye or both eyes of the subject are increased by at least 9s following administration of the therapeutically effective amount of the polypeptide of the present disclosure to the subject. In some embodiments, TBUT of one eye or both eyes of the subject are increased by at least 10s following administration of the therapeutically effective amount of the polypeptide of the present disclosure to the subject. In some embodiments, TBUT of one eye or both eyes of the subject are increased by at least 15s following administration of the therapeutically effective amount of the polypeptide of the present disclosure to the subject. In some embodiments, TBUT of one eye or both eyes of the subject are increased by at least 20s following administration of the therapeutically effective amount of the polypeptide of the present disclosure to the subject. In some embodiments, TBUT of one eye or both eyes of the subject are increased by at least 25s following administration of the therapeutically effective amount of the polypeptide of the present disclosure to the subject. In some embodiments, TBUT of one eye or both eyes of the subject are increased by at least 30s following administration of the therapeutically effective amount of the polypeptide of the present disclosure to the subject.

In some embodiments, one eye or both eyes of the subject of the present disclosure have a cornea fluorescein staining (CFS) score equal or higher than 5 prior to the treatment. In some embodiments, one eye or both eyes of the subject of the present disclosure have a CFS score equal or higher than 6 prior to the treatment. In some embodiments, one eye or both eyes of the subject of the present disclosure have a CFS score equal or higher than 7 prior to the treatment. In some embodiments, one eye or both eyes of the subject of the present disclosure have a CFS score equal or higher than 8 prior to the treatment. In some embodiments, one eye or both eyes of the subject of the present disclosure have a CFS score equal or higher than 9 prior to the treatment. In some embodiments, one eye or both eyes of the subject of the present disclosure have a CFS score equal or higher than 10 prior to the treatment.

In some embodiments, the CFS score of one eye of the subject is decreased following administration of the therapeutically effective amount of the polypeptide of the present disclosure to the subject. In some embodiments, the CFS scores of both eyes of the subject are decreased following administration of the therapeutically effective amount of the polypeptide of the present disclosure to the subject. In some embodiments, CFS scores of one or both eyes of the subject are decreased by at least 0.5 following administration of the therapeutically effective amount of the polypeptide of the present disclosure to the subject. In some embodiments, CFS scores of one or both eyes of the subject are decreased by at least 1 following administration of the therapeutically effective amount of the polypeptide of the present disclosure to the subject. In some embodiments, CFS scores of one or both eyes of the subject are decreased by at least 2 following administration of the therapeutically effective amount of the polypeptide of the present disclosure to the subject. In some embodiments, CFS scores of one or both eyes of the subject are decreased by at least 3 following administration of the therapeutically effective amount of the polypeptide of the present disclosure to the subject, In some embodiments, CFS scores of one or both eyes of the subject are decreased by at least 4 following administration of the therapeutically effective amount of the polypeptide of the present disclosure to the subject. In some embodiments, CFS scores of one or both eyes of the subject are decreased by at least 5 following administration of the therapeutically effective amount of the polypeptide of the present disclosure to the subject. In some embodiments, CFS scores of one or both eyes of the subject are decreased by at least 6 following administration of the therapeutically effective amount of the polypeptide of the present disclosure to the subject. In some embodiments, CFS scores of one or both eyes of the subject are decreased by at least 7 following administration of the therapeutically effective amount of the polypeptide of the present disclosure to the subject. In some embodiments, CFS scores of one or both eyes of the subject are decreased by at least 8 following administration of the therapeutically effective amount of the polypeptide of the present disclosure to the subject. In some embodiments, CFS scores of one or both eyes of the subject are decreased by at least 9 following administration of the therapeutically effective amount of the polypeptide of the present disclosure to the subject. In some embodiments, CFS scores of one or both eyes of the subject are decreased by at least 10 following administration of the therapeutically effective amount of the polypeptide of the present disclosure to the subject.

In some embodiments, the treatment comprising administrating a therapeutically effective amount of the polypeptide of the present disclosure to one affected eye or eye tissue of the subject. In some embodiments, the methods comprising administrating a therapeutically effective amount of the polypeptide of the present disclosure to both eyes or eye tissues of the subject. In some embodiments, the therapeutically effective amount of the polypeptide of the present disclosure is from about 0.1 μg to about 100 μg per eye, or from about 0.1 μg to about 50 μg per eye, or from about 0.1 μg to about 20 μg per eye, or from about 0.1 μg to about 10 μg per eye, or from about 0.5 μg to about 50 μg per eye, or from about 0.5 μg to about 20 μg per eye, or from about 0.5 μg to about 10 μg per eye, or from about 1 μg to about 10 μg per eye.

In some embodiments, the dosage for one eye of the subject can be about 1 to about 5 drops of the ophthalmic formulation of the present disclosure. In some embodiments, the dosage for one eye of the subject can be 1 drop of the ophthalmic formulation of the present disclosure. In some embodiments, the dosage for one eye of the subject can be 2 drops of the ophthalmic formulation of the present disclosure. In some embodiments, the dosage for one eye of the subject can be 3 drops of the ophthalmic formulation of the present disclosure. In some embodiments, the dosage for one eye of the subject can be 4 drops of the ophthalmic formulation of the present disclosure. In some embodiments, the dosage for one eye of the subject can be 5 drops of the ophthalmic formulation of the present disclosure. In some embodiments, each drop corresponds to about 10 μL to about 150 μL. In some embodiments, each drop corresponds to about 20 μL to about 70 μL.

In some embodiments, the method comprises administrating a therapeutically effective amount of the ophthalmic formulation in each eye of the subject once daily. In some embodiments, the method comprises administrating a therapeutically effective amount of the ophthalmic formulation in each eye of the subject twice daily. In some embodiments, the method comprises administrating a therapeutically effective amount of the ophthalmic formulation in each eye of the subject three or more times daily. In some embodiments, the method comprises administrating a therapeutically effective amount of the ophthalmic formulation in each eye of the subject every two days. In some embodiments, the method comprises administrating a therapeutically effective amount of the ophthalmic formulation in each eye of the subject every three days. In some embodiments, the method comprises administrating a therapeutically effective amount of the ophthalmic formulation in each eye of the subject weekly.

In some embodiments, the method comprises administrating one or more drops of the ophthalmic formulation of the present disclosure in each eye of the subject daily. In some embodiments, the method comprises administrating one to more drops of the ophthalmic formulation in each eye of the subject 2, 3, 4, 8, 12, 18 or 24 times daily. In some embodiments, the method comprises administrating one or more drops of the ophthalmic formulation of the present disclosure in each eye of the subject every two days.

In some embodiments, the polypeptide of the present disclosure is administrated in combination with one or more additional therapeutics. In some embodiments, therapeutically effective amount of the polypeptide of the present disclosure can be administered simultaneously with the additional therapeutics. In some embodiments, therapeutically effective amount of the polypeptide of the present disclosure can be administered prior to the administration of the additional therapeutics. In some embodiments, therapeutically effective amount of the polypeptide of the present disclosure can be administered after the administration of the additional therapeutics. In some embodiments, the additional therapeutics include but are not limited to artificial tear, antibiotic, antiviral agent, anti-fungal agent, anti-protozoal agent, anti-inflammatory drug, antiallergic agent, anesthetic, analgesic, intraocular pressure-lowering agent, immunoregulator, anti-oxidant, enzyme inhibitor, protease, peptidase, cytokine inhibitor, vitamin and mineral.

In some embodiment, therapeutically effective amount of the polypeptide of the present disclosure is administered at the same time, or separately during the treatment period for which a subject receives immunosuppressive therapies, such as azathioprine, cyclophosphoramide, methotrextate, antimalarial drugs, mycophenolan mofetile, daclizumab, intravenous immunoglobin therapy, and the like. In some embodiment, therapeutically effective amount of the polypeptide of the present disclosure of the invention is administered at the same time or separately during the treatment period for which a subject receives other anti-inflammatory treatments, such as cyclosporin A, corticosteroids, NSAIDSI asprin, doxycycline, and the like. In some embodiments, therapeutically effective amount of the polypeptide of the present disclosure of the invention is administered at the same time or separately during the treatment period for which a subject receives hormone therapy, and the like. In some embodiments, therapeutically effective amount of the polypeptide of the present disclosure of the invention is administered at the same time or separately during the treatment period for which a subject receives anti-allergy therapy, palliative care for dry eye including artificial tears or artificial saliva, muscarinic M3 receptor agonists to increase aqueous secretions, autologous scrum, sodium hyaluronate drops, and the like.

The present disclosure also contemplates the artificial polypeptide of formula (I), (II), (III), (IV), (V) or (VI), or a mutant of artificial polypeptide of (I), (II), (III), (IV) or (V), for use in therapy. In some embodiments, the present disclosure also contemplates the artificial polypeptide of formula (I), (II), (III), (IV), (V) or (VI), or a mutant of artificial polypeptide of (I), (II), (III), (IV) or (V), for use in treating or preventing dry eye (DE) or DE associated disorders, stimulating tears, stabilizing tear film or any combination thereof in a subject in need thereof. In some embodiments, the present disclosure also contemplates the polypeptide having at least 80% identity with any one selected from SEQ ID NO. 28, SEQ ID NOs. 57 and 62-74 or a fragment or variant thereof, for use in treating or preventing dry eye (DE) or DE associated disorders, stimulating tears, stabilizing tear film or any combination thereof in a subject in need thereof. In some embodiments, said DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia, Stevens-Johnson syndrome, ocular pemphigoid blepharitis marginal, eyelid-closure failure, corneal ulcer, blepharitis and eye redness, and the like.

The present disclosure also contemplates the use of an artificial polypeptide of formula (I), (II), (III), (IV), (V) or (VI), a mutant of artificial polypeptide of (I), (II), (III), (IV) or (V), or a polypeptide having at least 80% identity with any one selected from SEQ ID NO. 28, SEQ ID NOs. 57 and 62-74 or a fragment or variant thereof, for the preparation of a medicament for treating or preventing dry eye (DE) or DE associated disorders, stimulating tears, stabilizing tear film or any combination thereof in a subject in need thereof. In some embodiments, said DE or DE associated disorder is selected from the group consisting of dry eye syndrome, dysfunctional tear syndrome, keratoconjunctivitis sicca (KCS), lacrimal keratoconjunctivitis, aqueous tear-deficient DE, evaporative DE, Sjogren's syndrome DE, non-Sjogren's syndrome DE, conjunctivitis-associated DE, post-viral conjunctivitis DE, post-cataract surgery DE, VDT operation-associated DE, contact lens wearing-associated DE, environmental DE, corneal neovascularization DE, allergic DE, LASIK-induced neurotrophic epitheliopathy, hypolacrimation, xerophthalmia, Stevens-Johnson syndrome, ocular pemphigoid blepharitis marginal, eyelid-closure failure, corneal ulcer, blepharitis and eye redness, and the like.

### Kit

The present disclosure also provides a kit for preventing or treating DE or DE associated disorder, stimulating tears, stabilizing tear film or any combination thereof. In some embodiments, the kit comprises one or more the polypeptides of the present disclosure, and an instruction for using the kit. In some embodiments, the kit comprises one or more ophthalmic formulations described herein, and an instruction for using the kit.

The kits can comprise one or more containers that contain one or more ophthalmic formulations or the polypeptide of the present disclosure. The polypeptide of the present disclosure can be present in the container as a prepared pharmaceutical composition, or alternatively, the polypeptide of the present disclosure can be unformulated. In some embodiments, the kit can include the unformulated polypeptide of the present disclosure in a container that is separate from the pharmaceutically acceptable excipients. Prior to use, the polypeptide of the present disclosure is diluted or otherwise mixed with the pharmaceutically acceptable excipients.

In some embodiments, the kit provided herein also comprises instructions which describe the method for administering the ophthalmic formulations in such a way that one or more symptoms associated with DE or DE associated disorders are treated or prevented. In some embodiments, the instructions also describe the procedure for mixing the polypeptide of the present disclosure contained in the kit with pharmaceutically acceptable excipients.

In some embodiments, the container is configured to deliver the polypeptide of the present disclosure or ophthalmic formulation of the present disclosure. In some embodiments, the container comprises vial, dropper, bottle, tube, and syringe. In certain embodiments, the container is a dropper for administering eye drops. In other embodiments, the container is a tube for administering an ophthalmic gel or an ophthalmic ointment.

### Contact Lenses Care Product

According to the pathogenesis of dry eye (DE), the wearing of contact lenses can be regarded as one of the causes of dry eye. Specifically, wearing contact lens (contact lens wear, CL wear) is one of the factors that cause tear film instability. Other factors that cause tear film instability include vitamin A deficiency, eye allergies, topical use of preservatives, deficiency or instability of tear film lipid layer, and the like. Wearing contact lenses can also reduce lacrimal secretion by blocking the ocular reflex, resulting in tear hypertonicity.

Consequently, in another aspect, also provided herein is a contact lenses care product, which may be useful in preventing or alleviating contact lens wearing-associated DE. In some embodiments, the contact lenses care product comprises the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof. In some embodiments, the contact lenses care product is a solution for storing, protecting and/or cleansing contact lenses.

In some embodiments, the contact lenses care product further comprises one or more acceptable ingredients for contact lenses care product. In some embodiments, the one or more acceptable ingredients for contact lenses care product are selected from the group consisting of inorganic salts, boric acid, surfactants, moisturizers, preservatives, and solvents. In some embodiments, the contact lenses care product further comprises inorganic salts. In some embodiments, the contact lenses care product further comprises boric acid. In some embodiments, the contact lenses care product further comprises surfactants. In some embodiments, the contact lenses care product further comprises moisturizers. In some embodiments, the contact lenses care product further comprises preservatives. In some embodiments, the contact lenses care product further comprises solvents. In some embodiments, the contact lenses care product further comprises inorganic salts and solvents. In some embodiments, the contact lenses care product further comprises inorganic salts, boric acid and solvents. In some embodiments, the contact lenses care product further comprises inorganic salts, boric acid, surfactants and solvents. In some embodiments, the contact lenses care product further comprises inorganic salts, boric acid, moisturizers and solvents. In some embodiments, the contact lenses care product further comprises inorganic salts, boric acid, preservatives and solvents. In some embodiments, the contact lenses care product further comprises inorganic salts, boric acid, surfactants, moisturizers and solvents. In some embodiments, the contact lenses care product further comprises inorganic salts, boric acid, surfactants, preservatives and solvents. In some embodiments, the contact lenses care product further comprises inorganic salts, boric acid, surfactants, moisturizers, preservatives and solvents. In some embodiments, the contact lenses care product is consisting of the polypeptide of the present disclosure, inorganic salts, boric acid, surfactants, moisturizers, preservatives, and solvents.

In some embodiments, the inorganic salts comprise sodium chloride, potassium chloride, sodium dihydrogen phosphate, disodium hydrogen phosphate, sodium carbonate, sodium borate, or any combination thereof. In some embodiments, the inorganic salts comprise sodium chloride. In some embodiments, the inorganic salts comprise potassium chloride. In some embodiments, the inorganic salts comprise sodium dihydrogen phosphate, disodium hydrogen phosphate or a combination thereof. In some embodiments, the inorganic salts comprise sodium carbonate. In some embodiments, the inorganic salts comprise sodium borate. In some embodiments, the inorganic salts comprise sodium chloride, sodium borate, or both. In some embodiments, the inorganic salts comprise sodium chloride and sodium borate. In some embodiments, the inorganic salts are consisting of sodium chloride and sodium borate.

In some embodiments, the surfactants comprise a zwitterionic surfactant, a nonionic surfactant, or both. In some embodiments, the surfactants comprise a zwitterionic surfactant. In some embodiments, the surfactants comprise a nonionic surfactant. In some embodiments, the surfactants comprise a zwitterionic surfactant and a nonionic surfactant. Examples of zwitterionic surfactants include, but not limited to lecithin. Examples of a nonionic surfactant include, but not limited to sorbitan fatty acid esters (Spans), polyoxyethylene sorbitan fatty acid esters (Tweens), polyoxyethylene fatty acid esters (Myri), polyoxyethylene fatty alcohol ethers (Brij) and polyethylene-polypropylene glycols (e.g., poloxamers).

In some embodiments, the moisturizers comprise hyaluronic acid or a salt thereof. In some embodiments, the moisturizers comprise hyaluronic acid. In some embodiments, the moisturizers comprise a salt of hyaluronic acid, such as sodium hyaluronate. In some embodiments, the moisturizers comprise hyaluronic acid and a salt of hyaluronic acid, such as sodium hyaluronate. In some embodiments, the moisturizers are consisting of hyaluronic acid and a salt of hyaluronic acid, such as sodium hyaluronate.

In some embodiments, the preservatives comprise an organic mercuric compound, a quaternary ammonium salt, an alcohol, an ester, an acid, or any combination thereof. In some embodiments, the preservatives comprise an organic mercuric compound. In some embodiments, the preservatives comprise a quaternary ammonium salt. In some embodiments, the preservatives comprise an alcohol. In some embodiments, the preservatives comprise an ester. In some embodiments, the preservatives comprise an acid, In some embodiments, the preservatives comprise at least two of an organic mercuric compound, a quaternary ammonium salt, an alcohol, an ester, and an acid. Examples of organic mercuric compounds include, but not limited to phenylmercuric nitrate, phenylmercuric acetate, thimerosal, mercuric oxycyanide, and the like. Examples of quaternary ammonium salts include, but not limited to benzalkonium chloride, benzalkonium bromide, chlorhexidine, and the like. Examples of alcohols include, but not limited to trichlorobutanol, phenethyl alcohol, and the like. Examples of esters include, but not limited to methylparaben, ethylparaben, propylparaben, and the like Examples of acids include, but not limited to sorbic acid.

In some embodiments, the solvents comprise water. In some embodiments, the solvents are consisting of water.

In another aspect, also provided herein is a method for preparing a contact lenses care product, comprising the step of combining the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof with one or more acceptable ingredients for contact lenses care product.

In another aspect, also provided herein is use of the polypeptide of the present disclosure, e.g., an artificial polypeptide of formula (I), (II), (III), (IV), (V) or (VI), a mutant of an artificial polypeptide of formula (I), (II), (III), (IV) or (V), or a polypeptide having at least 70% identity (e.g., at least 80% identity) with any one selected from SEQ ID NO. 28, SEQ ID NOs. 57 and 62-74 or a fragment or variant thereof for the preparation of a contact lenses care product.

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

### EXAMPLES

### Example 1: Effects of the Polypeptide of the Present Disclosure on Treatment of Mild DE

Effects of exemplary polypeptide of the present disclosure (SEQ ID NO. 29, also referred as “CF-04” hereinafter) on treating DE were evaluated in Rhesus macaque as described below Briefly, 6 Rhesus Monkeys (5 females and 1 male, weight 9-13 kg) at ages of 12-23 (equivalent to 40-70 years of human) with any eye of the two eyes met the following criteria of mild DE within half year were enrolled in the study:
Cornea fluorescein staining (CFS) score: 5-10; or
Tear break-up time (TBUT): <5 seconds.

After enrollment, the monkeys were divided into two groups, with 2 in the placebo group and 4 in the treatment group Before treatment, the animals were subject to baseline examination on day day-13 and day-5. Then, the monkeys were administrated with eye drops of placebo (saline) or CF-04 (10 μM, dissolved in saline) from day 0 to day 13 for 14 consecutive days (two drops per eye, twice per day), followed by 18 days of wash-out period Clinical observation and food intake of the monkeys were also observed from day-13 to day 14.

### 1. Cornea Fluorescein Staining Score

Cornea fluorescein staining (CFS) scores indicates ocular surface damage of the eye, which has been used to assess DE, CFS scores were evaluated on day −5 and day 14 in each eye of the monkeys by using 2% sodium fluorescein solution Scores in C, S, T, I, N regions of the cornea were graded, respectively, and then the CFS score of the eye was calculated as sum of the scores of the five regions Table 1 shows the CFS scores of the monkeys in both placebo group and treatment group.

TABLE 1
CFS scores of monkeys with the treatment of placebo and CF-04.

| | Animal | | | Day of | | | | | | | |
|---|---|---|---|---|---|---|---|---|---|---|---|
| Group | ID | Eye | Age | Study | C | S | T | I | N | Total | Diagnosis |
| Placebo | 1 | OD | 19 | Baseline | 0 | 1 | 0 | 2 | 2 | 5 | Mild DE |
| (Saline) | 1 | OD | 19 | Day 14 | 1 | 0 | 0 | 3 | 0 | 4 | |
| | 1 | OD | 19 | Wash out | — | — | — | — | — | — | Not examined |
| | 1 | OS | 19 | Baseline | 0 | 0 | 0 | 0 | 0 | 0 | |
| | 1 | OS | 19 | Day 14 | 0 | 0 | 3 | 3 | 0 | 6 | |
| | 1 | OS | 19 | Wash out | — | — | — | — | — | — | Not examined |
| | 2 | OD | 17 | Baseline | 2 | 0 | 3 | 0 | 3 | 8 | Mild DE |
| | 2 | OD | 17 | Day 14 | 2 | 0 | 0 | 3 | 2 | 7 | Mild DE |
| | 2 | OD | 17 | Wash out | 1 | 0 | 0 | 3 | 3 | 7 | |
| | 2 | OS | 17 | Baseline | 3 | 0 | 1 | 1 | 0 | 4 | |
| | 2 | OS | 17 | Day 14 | 2 | 0 | 3 | 0 | 1 | 6 | |
| | 2 | OS | 17 | Wash out | 2 | 0 | 3 | 2 | 0 | 7 | |
| CF-04 | 3 | OD | 21 | Baseline | 0 | 0 | 2 | 3 | 0 | 5 | Mild DE |
| (10 μg/mL) | 3 | OD | 21 | Day 14 | 0 | 0 | 2 | 0 | 0 | 2 | Improved |
| | 3 | OD | 21 | Wash out | 0 | 0 | 1 | 3 | 0 | 4 | |
| | 3 | OS | 21 | Baseline | / | / | / | / | / | / | |
| | 3 | OS | 21 | Day 14 | / | / | / | / | / | / | |
| | 3 | OS | 21 | Wash out | / | / | / | / | / | / | |
| | 4 | OD | 20 | Baseline | 0 | 0 | 2 | 3 | 0 | 5 | Mild DE |
| | 4 | OD | 20 | Day 14 | 0 | 0 | 0 | 0 | 2 | 2 | Improved |
| | 4 | OD | 20 | Wash out | 0 | 0 | 0 | 3 | 2 | 5 | |
| | 4 | OS | 20 | Baseline | 2 | 0 | 0 | 3 | 1 | 6 | Mild DE |
| | 4 | OS | 20 | Day 14 | 0 | 0 | 2 | 0 | 0 | 2 | Improved |
| | 4 | OS | 20 | Wash out | 0 | 0 | 0 | 3 | 2 | 5 | |
| | 5 | OD | 12 | Baseline | 0 | 0 | 0 | 2 | 0 | 2 | |
| | 5 | OD | 12 | Day 14 | 2 | 0 | 0 | 0 | 0 | 2 | |
| | 5 | OD | 12 | Wash out | 2 | 0 | 0 | 2 | 2 | 6 | |
| | 5 | OS | 12 | Baseline | 0 | 0 | 3 | 3 | 0 | 6 | Mild DE |
| | 5 | OS | 12 | Day 14 | 1 | 0 | 2 | 0 | 0 | 3 | Improved |
| | 5 | OS | 12 | Wash out | 2 | 0 | 0 | 3 | 0 | 5 | |
| | 6 | OD | 15 | Baseline | 0 | 0 | 1 | 3 | 2 | 6 | Mild DE |
| | 6 | OD | 15 | Day 14 | 0 | 0 | 0 | 0 | 0 | 0 | Improved |
| | 6 | OD | 15 | Wash out | — | — | — | — | — | — | Not examined |
| | 6 | OS | 15 | Baseline | 0 | 0 | 3 | 2 | 0 | 5 | |
| | 6 | OS | 15 | Day 14 | 0 | 0 | 1 | 3 | 0 | 4 | |
| | 6 | OS | 15 | Wash out | — | — | — | — | — | — | Not examined |

OD: right eye; OS: left eye.
/: not applicable due to atrophy.

It can be seen from Table 1 that, in CF-04 group, administration of the polypeptide of the present disclosure decreased the CFS scores of the eyes, by at least 3 (OD of No. 3, OD and OS of No. 4, OS of No. 5 and OD of No. 6) After wash-out period, CFS scores of the eyes in CF-04 group increased to levels before treatment, indicating efficacy of CF-04 during the entire treatment period. However, CFS scores of DE eyes in placebo group did not show significant decrease (OD of No. 1 and OD of No. 2).

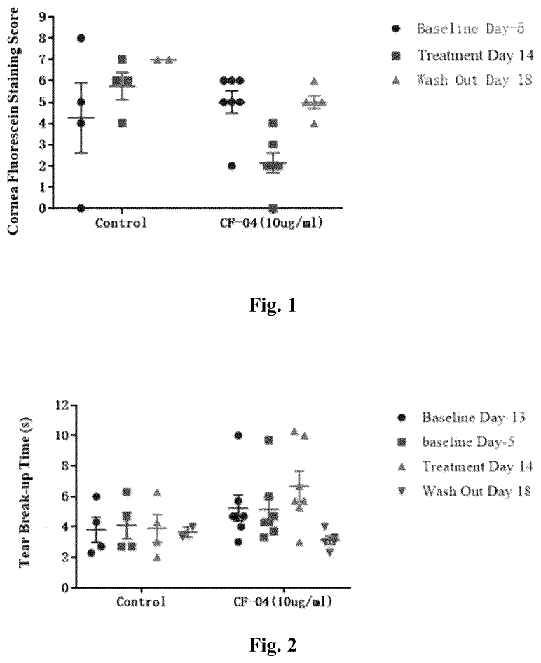

FIG. 1 also illustrates effects of the polypeptide of the present disclosure on CFS scores according to the data from Table 1. As shown in FIG. 1, on day 14 (red dots), the average CFS scores of all tested eyes in placebo group did not exhibit significant change compared to the baseline tested on day-5 (blue dots) (p=0.4287) However, in CF-04 group, the average CFS scores was significantly decreased on day 14 (red dots) as compared to day −5 (blue dots) (from 5.00 to 2.14, p=0.0016), indicating the administration of the polypeptide of the present disclosure improved ocular surface damage and alleviated the symptoms of DE After wash-out period, CFS scores of the eyes in CF-04 group increased to levels before treatment, further suggesting efficacy of CF-04 during the entire treatment period.

Meanwhile, administration of CF-04 did not affect the food intake and other clinical observations of the animals (data not shown), indicating safety of the polypeptide of the present disclosure.

### 2. Tear Break-Up Time (TBUT)

Tear break-up time (TBUT) indicates the stability of tear film, which is broadly used to assess DE In this study, TBUT was respectively evaluated on day −13, day −5 and day 14 (3 tests on each day) The result is as shown in Table 2.

TABLE 2
TBUT of monkeys with the treatment of placebo and CF-04

| | Animal | | | Day of | | | | | |
|---|---|---|---|---|---|---|---|---|---|
| Group | ID | Eye | Age | Study | 1 | 2 | 3 | Average | Diagnosis |
| Placebo | 1 | OD | 19 | Baseline1 | 5 | 8 | 5 | 6.0 | |
| (Saline) | 1 | OD | 19 | Baseline2 | 7 | 6 | 6 | 6.3 | |
| | 1 | OD | 19 | Day 14 | 7 | 7 | 5 | 6.3 | |
| | 1 | OD | 19 | Wash out | — | — | — | — | Not examined |
| | 1 | OS | 19 | Baseline1 | 5 | 3 | 5 | 4.3 | |
| | 1 | OS | 19 | Baseline2 | 4 | 6 | 4 | 4.7 | Mild DE |
| | 1 | OS | 19 | Day 14 | 4 | 4 | 5 | 4.3 | Mild DE |
| | 1 | OS | 19 | Wash out | — | — | — | — | Not examined |
| | 2 | OD | 17 | Baseline1 | 3 | 2 | 2 | 2.3 | |
| | 2 | OD | 17 | Baseline2 | 2 | 3 | 3 | 2.7 | Mild DE |
| | 2 | OD | 17 | Day 14 | 2 | 4 | 3 | 3.0 | Mild DE |
| | 2 | OD | 17 | Wash out | 3 | 4 | 3 | 3.3 | |
| | 2 | OS | 17 | Baseline1 | 4 | 2 | 2 | 2.7 | |
| | 2 | OS | 17 | Baseline2 | 3 | 3 | 2 | 2.7 | Mild DE |
| | 2 | OS | 17 | Day 14 | 2 | 2 | 2 | 2.0 | Mild DE |
| | 2 | OS | 17 | Wash out | 3 | 5 | 4 | 4.0 | |
| CF-04 | 3 | OD | 21 | Baseline1 | 6 | 4 | 4 | 4.7 | |
| (10 μg/mL) | 3 | OD | 21 | Baseline2 | 4 | 5 | 5 | 4.7 | Mild DE |
| | 3 | OD | 21 | Day 14 | 11 | 8 | 11 | 10.0 | Improved |
| | 3 | OD | 21 | Wash out | 3 | 2 | 2 | 2.3 | |
| | 3 | OS | 21 | Baseline1 | / | / | / | / | |
| | 3 | OS | 21 | Baseline2 | / | / | / | / | |
| | 3 | OS | 21 | Day 14 | / | / | / | / | |
| | 3 | OS | 21 | Wash out | / | / | / | / | |
| | 4 | OD | 20 | Baseline1 | 8 | 5 | 4 | 5.7 | |
| | 4 | OD | 20 | Baseline2 | 6 | 6 | 6 | 6.0 | |
| | 4 | OD | 20 | Day 14 | 7 | 5 | 5 | 5.7 | |
| | 4 | OD | 20 | Wash out | 2 | 4 | 3 | 3.0 | |
| | 4 | OS | 20 | Baseline1 | 2 | 3 | 4 | 3.0 | |
| | 4 | OS | 20 | Baseline2 | 4 | 3 | 4 | 3.7 | Mild DE |
| | 4 | OS | 20 | Day 14 | 3 | 3 | 3 | 3.0 | |
| | 4 | OS | 20 | Wash out | 4 | 3 | 3 | 3.3 | |
| | 5 | OD | 12 | Baseline1 | 5 | 3 | 4 | 4.0 | |
| | 5 | OD | 12 | Baseline2 | 3 | 4 | 3 | 3.3 | Mild DE |
| | 5 | OD | 12 | Day 14 | 6 | 6 | 8 | 6.7 | Improved |
| | 5 | OD | 12 | Wash out | 3 | 5 | 4 | 4.0 | |
| | 5 | OS | 12 | Baseline1 | 6 | 3 | 5 | 4.7 | |
| | 5 | OS | 12 | Baseline2 | 5 | 4 | 4 | 4.3 | Mild DE |
| | 5 | OS | 12 | Day 14 | 5 | 6 | 6 | 5.7 | Improved |
| | 5 | OS | 12 | Wash out | 2 | 4 | 3 | 3.0 | |
| | 6 | OD | 15 | Baseline1 | 12 | 9 | 9 | 10.0 | |
| | 6 | OD | 15 | Baseline2 | 9 | 10 | 10 | 9.7 | |
| | 6 | OD | 15 | Day 14 | 9 | 11 | 11 | 10.3 | |
| | 6 | OD | 15 | Wash out | — | — | — | — | Not examined |
| | 6 | OS | 15 | Baseline1 | 3 | 5 | 6 | 4.7 | |
| | 6 | OS | 15 | Baseline2 | 5 | 4 | 4 | 4.3 | Mild DE |
| | 6 | OS | 15 | Day 14 | 5 | 5 | 6 | 5.3 | Improved |
| | 6 | OS | 15 | Wash out | — | — | — | — | Not examined |

OD: right eye; OS: left eye.
/: not applicable due to atrophy.

As can be seen from Table 2, in CF-04 group, administration of the polypeptide of the present disclosure increased the TBUT of DE eyes (OD of No. 3, OD and OS of No. 5 and OS of No. 6) to longer than 5 seconds. However, in control group, placebo was not able to increase the TUBT of DE eyes (OS of No. 1, OD and OS of No. 2).

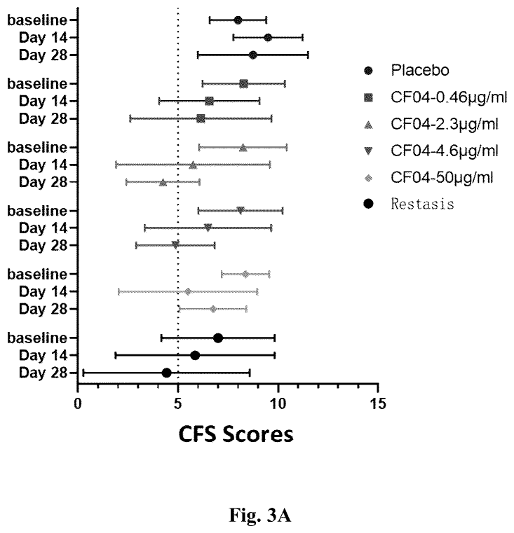

FIG. 2 also illustrates effects of the polypeptide of the present disclosure on TBUT according to the data from Table 2. As shown in FIG. 2, on day 14, in placebo group, the TBUT of all tested eyes was not significantly different from baseline 2 (day −5) (p=0.8804). In CF-04 group, although on day 14, the TBUT of all tested eyes was not significantly different compared to baseline 2 (p=0.2596), the mean TBUT was increased by more than 1 second (5.143±2.183 vs 6.671±2.630), suggesting administration of the polypeptides of the present disclosure can potentially stabilize tear film and alleviate the symptom of DE After wash-out period, CFS scores of the eyes in CF-04 group increased to levels before treatment, indicating efficacy of CF-04 during the entire treatment period.

### Example 2: Effects of the Polypeptide of the Present Disclosure on the Treatment of DE

Effects of exemplary polypeptide of the present disclosure (SEQ ID NO. 29, also referred as CF-04 hereinafter) on treating DE were evaluated in Rhesus Macaque as described below Briefly, 22 monkeys at ages of 12-24 (equivalent to 40-70 years of human) with any eye of the two eyes met the following criteria of DE within half year were enrolled in the study:
Cornea fluorescein staining (CFS) score: 5-10; and
Tear break-up time (TBUT): <5 seconds.

The monkeys were divided into six groups including placebo (saline, 2 monkeys), positive control (Restasis, 4 monkeys), CF-04 0.46 μg/ml (4 monkeys), CF-04 2.3 μg/ml (4 monkeys), CF-04 4.6 μg/ml (4 monkeys), and CF-04 50 μg/ml (4 monkeys) Briefly, animals were treated with 2 drops of placebo or CF-04 per eye, twice a day; for positive control group, animals were treated with 1 drop of Restasis per eye, twice a day, for 28 or 30 consecutive days Clinical observation and food intake of the monkeys were also observed on baseline, day 14 and the end of the administration (day 28 or day 30).

### 1. Cornea Fluorescein Staining Score

CFS scores were evaluated on baseline, day 14 and the end of the administration (day 28 or day 30) in each eye of the monkeys by using 2% sodium fluorescein solution Scores in C, S, T, I, N regions of the cornea were graded, respectively, and then the CFS score of the eye was calculated as sum of the scores of the five regions Table 3 shows the CFS scores of the monkeys in both placebo group and treatment group.

TABLE 3
CFS scores of monkeys with the treatment of CF-04.

| | | | Age | Study | | Scores | | | | | | |
|---|---|---|---|---|---|---|---|---|---|---|---|---|
| Group | Monkey ID | Gender | (yrs) | Day | Eye | C | S | T | I | N | Total | Note |
| Placebo | 4815 | Male | 22 | Baseline | OD | 2 | 1 | 3 | 2 | 1 | 9 | |
| Placebo | 4815 | Male | 22 | Day 14 | OD | 2 | 2 | 2 | 3 | 3 | 12 | |
| Placebo | 4815 | Male | 22 | Day 30 | OD | 2 | 2 | 2 | 2 | 2 | 10 | |
| Placebo | 4815 | Male | 22 | Baseline | OS | 1 | 3 | 2 | 1 | 1 | 8 | |
| Placebo | 4815 | Male | 22 | Day 14 | OS | 1 | 2 | 2 | 2 | 2 | 9 | |
| Placebo | 4815 | Male | 22 | Day 30 | OS | 3 | 2 | 3 | 2 | 2 | 12 | |
| Placebo | 6629 | Male | 24 | Baseline | OD | 1 | 1 | 2 | 0 | 2 | 6 | |
| Placebo | 6629 | Male | 24 | Day 14 | OD | 1 | 2 | 1 | 1 | 3 | 8 | |
| Placebo | 6629 | Male | 24 | Day 30 | OD | 1 | 1 | 2 | 1 | 1 | 6 | |
| Placebo | 6629 | Male | 24 | Baseline | OS | 1 | 2 | 1 | 3 | 2 | 9 | |
| Placebo | 6629 | Male | 24 | Day 14 | OS | 3 | 2 | 2 | 1 | 1 | 9 | |
| Placebo | 6629 | Male | 24 | Day 30 | OS | 1 | 2 | 2 | 1 | 1 | 7 | |
| CF-04 0.46 μg/ml | 115 | Male | 16 | Baseline | OD | 2 | 2 | 2 | 2 | 2 | 10 | |
| CF-04 0.46 μg/ml | 115 | Male | 16 | Day 14 | OD | 3 | 2 | 2 | 1 | 2 | 10 | |
| CF-04 0.46 μg/ml | 115 | Male | 16 | Day 28 | OD | 3 | 2 | 2 | 2 | 1 | 10 | |
| CF-04 0.46 μg/ml | 115 | Male | 16 | Baseline | OS | 1 | 2 | 2 | 2 | 2 | 9 | |
| CF-04 0.46 μg/ml | 115 | Male | 16 | Day 14 | OS | 2 | 2 | 2 | 2 | 1 | 9 | |
| CF-04 0.46 μg/ml | 115 | Male | 16 | Day 28 | OS | 2 | 3 | 1 | 2 | 2 | 10 | |
| CF-04 0.46 μg/ml | 2666 | Female | 16 | Baseline | OD | 2 | 2 | 2 | 2 | 2 | 10 | |
| CF-04 0.46 μg/ml | 2666 | Female | 16 | Day 14 | OD | 1 | 1 | 1 | 2 | 2 | 7 | |
| CF-04 0.46 μg/ml | 2666 | Female | 16 | Day 28 | OD | 2 | 1 | 1 | 1 | 2 | 7 | |
| CF-04 0.46 μg/ml | 2666 | Female | 16 | Baseline | OS | 2 | 2 | 2 | 2 | 2 | 10 | |
| CF-04 0.46 μg/ml | 2666 | Female | 16 | Day 14 | OS | 0 | 0 | 1 | 1 | 1 | 3 | |
| CF-04 0.46 μg/ml | 2666 | Female | 16 | Day 28 | OS | 0 | 0 | 0 | 0 | 1 | 1 | |
| CF-04 0.46 μg/ml | 5170 | Female | 22 | Baseline | OD | 1 | 2 | 2 | 2 | 1 | 8 | |
| CF-04 0.46 μg/ml | 5170 | Female | 22 | Day 14 | OD | 1 | 1 | 1 | 1 | 2 | 6 | |
| CF-04 0.46 μg/ml | 5170 | Female | 22 | Day 28 | OD | 0 | 0 | 1 | 1 | 0 | 2 | |
| CF-04 0.46 μg/ml | 5170 | Female | 22 | Baseline | OS | / | / | / | / | / | / | Eyeball |
| | | | | | | | | | | | | atrophy |
| CF-04 0.46 μg/ml | 5170 | Female | 22 | Day 14 | OS | / | / | / | / | / | / | |
| CF-04 0.46 μg/ml | 5170 | Female | 22 | Day 28 | OS | / | / | / | / | / | / | |
| CF-04 0.46 μg/ml | 9258 | Female | 13 | Baseline | OD | 1 | 1 | 1 | 1 | 2 | 6 | |
| CF-04 0.46 μg/ml | 9258 | Female | 13 | Day 14 | OD | 1 | 1 | 1 | 0 | 1 | 4 | |
| CF-04 0.46 μg/ml | 9258 | Female | 13 | Day 28 | OD | 2 | 1 | 1 | 1 | 1 | 6 | |
| CF-04 0.46 μg/ml | 9258 | Female | 13 | Baseline | OS | 1 | 1 | 1 | 1 | 1 | 5 | |
| CF-04 0.46 μg/ml | 9258 | Female | 13 | Day 14 | OS | 2 | 1 | 2 | 1 | 1 | 7 | |
| CF-04 0.46 μg/ml | 9258 | Female | 13 | Day 28 | OS | 2 | 1 | 2 | 1 | 1 | 7 | |
| CF-04 2.3 μg/ml | 2400 | Female | 18 | Baseline | OD | 2 | 2 | 2 | 2 | 2 | 10 | |
| CF-04 2.3 μg/ml | 2400 | Female | 18 | Day 14 | OD | 1 | 3 | 3 | 1 | 1 | 9 | |
| CF-04 2.3 μg/ml | 2400 | Female | 18 | Day 28 | OD | 2 | 1 | 2 | 1 | 1 | 7 | |
| CF-04 2.3 μg/ml | 2400 | Female | 18 | Baseline | OS | 2 | 2 | 2 | 2 | 2 | 10 | |
| CF-04 2.3 μg/ml | 2400 | Female | 18 | Day 14 | OS | 2 | 2 | 1 | 2 | 2 | 9 | |
| CF-04 2.3 μg/ml | 2400 | Female | 18 | Day 28 | OS | 1 | 1 | 1 | 1 | 2 | 6 | |
| CF-04 2.3 μg/ml | 3701 | Male | 18 | Baseline | OD | 1 | 2 | 1 | 2 | 2 | 8 | |
| CF-04 2.3 μg/ml | 3701 | Male | 18 | Day 14 | OD | 0 | 0 | 0 | 1 | 1 | 2 | |
| CF-04 2.3 μg/ml | 3701 | Male | 18 | Day 28 | OD | 0 | 0 | 1 | 1 | 1 | 3 | |
| CF-04 2.3 μg/ml | 3701 | Male | 18 | Baseline | OS | 1 | 3 | 3 | 2 | 1 | 10 | |
| CF-04 2.3 μg/ml | 3701 | Male | 18 | Day 14 | OS | 0 | 1 | 0 | 1 | 0 | 2 | |
| CF-04 2.3 μg/ml | 3701 | Male | 18 | Day 28 | OS | 0 | 0 | 1 | 1 | 1 | 3 | |
| CF-04 2.3 μg/ml | 8291 | Male | 12 | Baseline | OD | 1 | 1 | 1 | 1 | 1 | 5 | |
| CF-04 2.3 μg/ml | 8291 | Male | 12 | Day 14 | OD | 0 | 0 | 1 | 0 | 0 | 1 | |
| CF-04 2.3 μg/ml | 8291 | Male | 12 | Day 28 | OD | 0 | 0 | 0 | 2 | 1 | 3 | |
| CF-04 2.3 μg/ml | 8291 | Male | 12 | Baseline | OS | 1 | 1 | 1 | 1 | 1 | 5 | |
| CF-04 2.3 μg/ml | 8291 | Male | 12 | Day 14 | OS | 0 | 2 | 1 | 1 | 0 | 4 | |
| CF-04 2.3 μg/ml | 8291 | Male | 12 | Day 28 | OS | 0 | 0 | 0 | 0 | 0 | 2 | |
| CF-04 2.3 μg/ml | 13053 | Male | 13 | Baseline | OD | 2 | 1 | 3 | 1 | 3 | 10 | |
| CF-04 2.3 μg/ml | 13053 | Male | 13 | Day 14 | OD | 2 | 1 | 2 | 2 | 2 | 9 | |
| CF-04 2.3 μg/ml | 13053 | Male | 13 | Day 28 | OD | 2 | 0 | 2 | 1 | 1 | 6 | |
| CF-04 2.3 μg/ml | 13053 | Male | 13 | Baseline | OS | 2 | 2 | 2 | 1 | 1 | 8 | |
| CF-04 2.3 μg/ml | 13053 | Male | 13 | Day 14 | OS | 2 | 2 | 2 | 2 | 2 | 10 | |
| CF-04 2.3 μg/ml | 13053 | Male | 13 | Day 28 | OS | 1 | 0 | 2 | 0 | 1 | 4 | |
| CF-04 4.6 μg/ml | 1822 | Female | 18 | Baseline | OD | 2 | 1 | 2 | 2 | 1 | 8 | |
| CF-04 4.6 μg/ml | 1822 | Female | 18 | Day 14 | OD | 2 | 1 | 2 | 1 | 2 | 8 | |
| CF-04 4.6 μg/ml | 1822 | Female | 18 | Day 28 | OD | 1 | 0 | 2 | 1 | 1 | 5 | |
| CF-04 4.6 μg/ml | 1822 | Female | 18 | Baseline | OS | 2 | 1 | 2 | 1 | 2 | 8 | |
| CF-04 4.6 μg/ml | 1822 | Female | 18 | Day 14 | OS | 1 | 2 | 2 | 1 | 2 | 8 | |
| CF-04 4.6 μg/ml | 1822 | Female | 18 | Day 28 | OS | 0 | 1 | 1 | 2 | 1 | 5 | |
| CF-04 4.6 μg/ml | 1927 | Male | 19 | Baseline | OD | 1 | 1 | 1 | 1 | 1 | 5 | |
| CF-04 4.6 μg/ml | 1927 | Male | 19 | Day 14 | OD | 0 | 0 | 0 | 1 | 0 | 1 | |
| CF-04 4.6 μg/ml | 1927 | Male | 19 | Day 28 | OD | 0 | 0 | 0 | 1 | 1 | 2 | |
| CF-04 4.6 μg/ml | 1927 | Male | 19 | Baseline | OS | 1 | 1 | 1 | 1 | 1 | 5 | |
| CF-04 4.6 μg/ml | 1927 | Male | 19 | Day 14 | OS | 1 | 0 | 0 | 1 | 0 | 2 | |
| CF-04 4.6 μg/ml | 1927 | Male | 19 | Day 28 | OS | 0 | 0 | 1 | 1 | 1 | 3 | |
| CF-04 4.6 μg/ml | 2870 | Female | 16 | Baseline | OD | 2 | 2 | 2 | 2 | 2 | 10 | |
| CF-04 4.6 μg/ml | 2870 | Female | 16 | Day 14 | OD | 2 | 2 | 1 | 2 | 2 | 9 | |
| CF-04 4.6 μg/ml | 2870 | Female | 16 | Day 28 | OD | 1 | 1 | 1 | 2 | 2 | 7 | |
| CF-04 4.6 μg/ml | 2870 | Female | 16 | Baseline | OS | 2 | 2 | 2 | 2 | 2 | 10 | |
| CF-04 4.6 μg/ml | 2870 | Female | 16 | Day 14 | OS | 2 | 2 | 1 | 2 | 2 | 9 | N/A |
| CF-04 4.6 μg/ml | 2870 | Female | 16 | Day 28 | OS | 1 | 2 | 1 | 2 | 2 | 8 | |
| CF-04 4.6 μg/ml | 8033 | Male | 12 | Baseline | OD | 2 | 2 | 2 | 2 | 2 | 10 | |
| CF-04 4.6 μg/ml | 8033 | Male | 12 | Day 14 | OD | 1 | 1 | 0 | 3 | 2 | 7 | |
| CF-04 4.6 μg/ml | 8033 | Male | 12 | Day 28 | OD | 0 | 0 | 1 | 3 | 1 | 5 | |
| CF-04 4.6 μg/ml | 8033 | Male | 12 | Baseline | OS | 2 | 2 | 1 | 2 | 2 | 9 | |
| CF-04 4.6 μg/ml | 8033 | Male | 12 | Day 14 | OS | 1 | 2 | 2 | 2 | 1 | 8 | |
| CF-04 4.6 μg/ml | 8033 | Male | 12 | Day 28 | OS | 0 | 0 | 2 | 1 | 1 | 4 | |
| CF-04 50 μg/ml | 5 | Male | 24 | Baseline | OD | 0 | 0 | 2 | 3 | 2 | 7 | |
| CF-04 50 μg/ml | 5 | Male | 24 | Day 14 | OD | 0 | 0 | 0 | 3 | 2 | 5 | |
| CF-04 50 μg/ml | 5 | Male | 24 | Day 30 | OD | 0 | 1 | 2 | 2 | 2 | 7 | |
| CF-04 50 μg/ml | 5 | Male | 24 | Baseline | OS | 1 | 2 | 2 | 2 | 2 | 9 | |
| CF-04 50 μg/ml | 5 | Male | 24 | Day 14 | OS | 0 | 0 | 0 | 1 | 0 | 1 | |
| CF-04 50 μg/ml | 5 | Male | 24 | Day 30 | OS | 1 | 0 | 2 | 3 | 0 | 6 | |
| CF-04 50 μg/ml | 2870 | Female | 15 | Baseline | OD | 1 | 2 | 1 | 2 | 3 | 9 | |
| CF-04 50 μg/ml | 2870 | Female | 15 | Day 14 | OD | 1 | 2 | 1 | 2 | 3 | 9 | |
| CF-04 50 μg/ml | 2870 | Female | 15 | Day 30 | OD | 1 | 1 | 0 | 2 | 2 | 6 | |
| CF-04 50 μg/ml | 2870 | Female | 15 | Baseline | OS | 1 | 1 | 1 | 2 | 2 | 7 | |
| CF-04 50 μg/ml | 2870 | Female | 15 | Day 14 | OS | 1 | 1 | 1 | 1 | 2 | 6 | |
| CF-04 50 μg/ml | 2870 | Female | 15 | Day 30 | OS | 1 | 0 | 1 | 1 | 2 | 5 | |
| CF-04 50 μg/ml | 2930 | Female | 19 | Baseline | OD | 2 | 1 | 0 | 3 | 3 | 9 | |
| CF-04 50 μg/ml | 2930 | Female | 19 | Day 14 | OD | 0 | 1 | 1 | 3 | 0 | 5 | |
| CF-04 50 μg/ml | 2930 | Female | 19 | Day 30 | OD | 2 | 1 | 0 | 3 | 2 | 8 | |
| CF-04 50 μg/ml | 2930 | Female | 19 | Baseline | OS | 3 | 1 | 0 | 1 | 2 | 7 | |
| CF-04 50 μg/ml | 2930 | Female | 19 | Day 14 | OS | 0 | 0 | 0 | 0 | 1 | 1 | |
| CF-04 50 μg/ml | 2930 | Female | 19 | Day 30 | OS | 0 | 1 | 2 | 1 | 1 | 5 | |
| CF-04 50 μg/ml | 6707 | Male | 20 | Baseline | OD | 3 | 1 | 2 | 2 | 2 | 10 | |
| CF-04 50 μg/ml | 6707 | Male | 20 | Day 14 | OD | 2 | 2 | 3 | 2 | 2 | 11 | |
| CF-04 50 μg/ml | 6707 | Male | 20 | Day 30 | OD | 3 | 1 | 2 | 2 | 2 | 10 | |
| CF-04 50 μg/ml | 6707 | Male | 20 | Baseline | OS | 3 | 0 | 3 | 2 | 1 | 9 | |
| CF-04 50 μg/ml | 6707 | Male | 20 | Day 14 | OS | 0 | 1 | 1 | 2 | 2 | 6 | |
| CF-04 50 μg/ml | 6707 | Male | 20 | Day 30 | OS | 2 | 1 | 2 | 1 | 1 | 7 | |
| Restasis 0.05% | 1864 | Female | 20 | Baseline | OD | 1 | 1 | 3 | 2 | 1 | 8 | N/A |
| Restasis 0.05% | 1864 | Female | 20 | Day 14 | OD | 2 | 3 | 3 | 2 | 3 | 13 | |
| Restasis 0.05% | 1864 | Female | 20 | Day 30 | OD | 2 | 2 | 3 | 3 | 2 | 12 | |
| Restasis 0.05% | 1864 | Female | 20 | Baseline | OS | 0 | 2 | 2 | 2 | 1 | 7 | |
| Restasis 0.05% | 1864 | Female | 20 | Day 14 | OS | 0 | 1 | 1 | 2 | 1 | 5 | |
| Restasis 0.05% | 1864 | Female | 20 | Day 30 | OS | 1 | 0 | 2 | 2 | 1 | 6 | |
| Restasis 0.05% | 5170 | Female | 21 | Baseline | OD | 1 | 2 | 2 | 2 | 1 | 8 | |
| Restasis 0.05% | 5170 | Female | 21 | Day 14 | OD | 1 | 2 | 1 | 1 | 1 | 6 | |
| Restasis 0.05% | 5170 | Female | 21 | Day 30 | OD | 1 | 2 | 1 | 1 | 1 | 6 | |
| Restasis 0.05% | 5170 | Female | 21 | Baseline | OS | / | / | / | / | / | / | |
| Restasis 0.05% | 5170 | Female | 21 | Day 14 | OS | / | / | / | / | / | / | |
| Restasis 0.05% | 5170 | Female | 21 | Day 30 | OS | / | / | / | / | / | / | |
| Restasis 0.05% | 6669 | Male | 23 | Baseline | OD | 1 | 1 | 1 | 2 | 2 | 7 | |
| Restasis 0.05% | 6669 | Male | 23 | Day 14 | OD | 1 | 1 | 2 | 1 | 1 | 6 | |
| Restasis 0.05% | 6669 | Male | 23 | Day 30 | OD | 0 | 0 | 0 | 0 | 0 | 0 | |
| Restasis 0.05% | 6669 | Male | 23 | Baseline | OS | 0 | 0 | 0 | 1 | 0 | 1 | Not |
| | | | | | | | | | | | | enrolled |
| Restasis 0.05% | 6669 | Male | 23 | Day 14 | OS | 1 | 0 | 0 | 1 | 0 | 2 | |
| Restasis 0.05% | 6669 | Male | 23 | Day 30 | OS | 0 | 1 | 1 | 1 | 1 | 4 | |
| Restasis 0.05% | 7983 | Male | 12 | Baseline | OD | 2 | 2 | 1 | 3 | 2 | 10 | |
| Restasis 0.05% | 7983 | Male | 12 | Day 14 | OD | 3 | 2 | 0 | 2 | 1 | 8 | |
| Restasis 0.05% | 7983 | Male | 12 | Day 30 | OD | 0 | 1 | 1 | 1 | 0 | 3 | |
| Restasis 0.05% | 7983 | Male | 12 | Baseline | OS | 1 | 2 | 3 | 1 | 1 | 8 | |
| Restasis 0.05% | 7983 | Male | 12 | Day 14 | OS | 0 | 0 | 0 | 0 | 1 | 1 | |
| Restasis 0.05% | 7983 | Male | 12 | Day 30 | OS | 0 | 0 | 0 | 0 | 0 | 0 | |

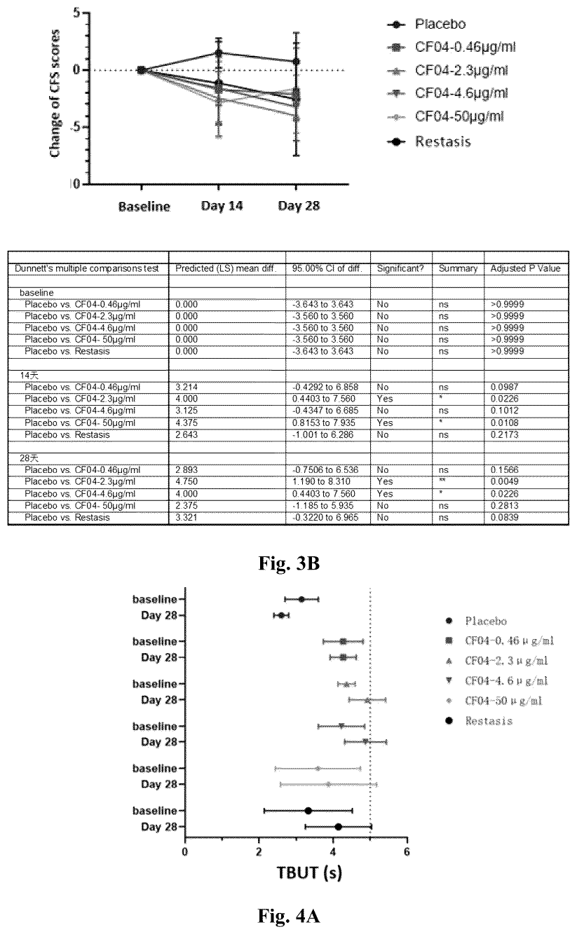

The scores were collected and analyzed by ANOVA, FIG. 3A-3B illustrates effects of the polypeptides of the present disclosure on CFS scores according to the data from Table 3. As shown in FIG. 3A, on day 14 and day 28, the average CFS scores of placebo group did not exhibit significant change as compared to the baseline, CFS scores of animals treated with 0.46 μg/ml CF-04 did not show significant change on day 14 (→=3.214, p=0.0987) or day 28 (Δ=2.893, p=0.1566) as compared to the baseline. Treatment of 2.3 g/ml CF-04 significantly decreased the CFS scores on day 14 (Δ=4.000, p=0.0226) and further decreased the scores on day 28 (Δ=4.750, p=0.0049), as compared to the baseline. Treatment of 4.6 μg/ml CF-04 did not show significant effect on the CFS scores on day 14 (Δ=3.125, p=0.1012) but significantly decreased the CFS scores on day 28 (Δ=4.000, p=0.0226), as compared to the baseline. Treatment of 50 μg/ml CF-04 significantly decreased the CFS scores on day 14 but did not further decrease the CFS scores on day 28 (Δ=4.000, p=0.0226) as compared to the baseline.

FIG. 3B further illustrates CFS scores collected from animals of different groups. As can be seen from FIG. 3B, on day 14 and day 28, treatment with 2.3 μg/ml CF-04 significantly decreased the CFS scores of eyes of animals as compared to those of eyes in placebo group.

### 2. Tear Break-Up Time (TBUT)

TBUT of the animals in each group was evaluated on baseline, day 14 and day 28, as shown in Table 4.

TABLE 4
TBUT of animals treated with CF-04

| | | | Age | Stdudy | | TBUT (s) | | | |
|---|---|---|---|---|---|---|---|---|---|
| Group | Monkey ID | Gender | (yrs) | Day | Eye | 1st | 2nd | 3rd | Mean |
| Placebo | 4815 | Male | 22 | Baseline1 | OD | 3 | 3 | 4 | 3.3 |
| Placebo | 4815 | Male | 22 | Baseline2 | OD | 2 | 4 | 3 | 3.0 |
| Placebo | 4815 | Male | 22 | D 14 | OD | 3 | 2 | 2 | 2.3 |
| Placebo | 4815 | Male | 22 | D 30 | OD | 2 | 3 | 3 | 2.7 |
| Placebo | 4815 | Male | 22 | Baseline1 | OS | 2 | 2 | 3 | 2.3 |
| Placebo | 4815 | Male | 22 | Baseline2 | OS | 3 | 2 | 3 | 2.7 |
| Placebo | 4815 | Male | 22 | D 14 | OS | 2 | 2 | 2 | 2.0 |
| Placebo | 4815 | Male | 22 | D 30 | OS | 3 | 2 | 2 | 2.3 |
| Placebo | 6629 | Male | 24 | Baseline1 | OD | 3 | 3 | 3 | 3.0 |
| Placebo | 6629 | Male | 24 | Baseline2 | OD | 3 | 3 | 5 | 3.7 |
| Placebo | 6629 | Male | 24 | D 14 | OD | 2 | 2 | 2 | 2.0 |
| Placebo | 6629 | Male | 24 | D 30 | OD | 3 | 3 | 2 | 2.7 |
| Placebo | 6629 | Male | 24 | Baseline1 | OS | 4 | 4 | 3 | 3.7 |
| Placebo | 6629 | Male | 24 | Baseline2 | OS | 4 | 3 | 3 | 3.3 |
| Placebo | 6629 | Male | 24 | D 14 | OS | 3 | 2 | 2 | 2.3 |
| Placebo | 6629 | Male | 24 | D 30 | OS | 2 | 3 | 3 | 2.7 |
| CF-04 0.46 μg/ml | 115 | Male | 16 | Baseline | OD | 4 | 4 | 5 | 4.3 |
| CF-04 0.46 μg/ml | 115 | Male | 16 | Day 28 | OD | 5 | 3 | 4 | 4 |
| CF-04 0.46 μg/ml | 115 | Male | 16 | Baseline | OS | 5 | 4 | 4 | 4.3 |
| CF-04 0.46 μg/ml | 115 | Male | 16 | Day 28 | OS | 4 | 5 | 4 | 4.3 |
| CF-04 0.46 μg/ml | 2666 | Female | 16 | Baseline | OD | 4 | 4 | 4 | 4 |
| CF-04 0.46 μg/ml | 2666 | Female | 16 | Day 28 | OD | 5 | 4 | 4 | 4.3 |
| CF-04 0.46 μg/ml | 2666 | Female | 16 | Baseline | OS | 5 | 4 | 4 | 4.3 |
| CF-04 0.46 μg/ml | 2666 | Female | 16 | Day 28 | OS | 6 | 5 | 4 | 5 |
| CF-04 0.46 μg/ml | 5170 | Female | 22 | Baseline | OD | 4 | 3 | 3 | 3.3 |
| CF-04 0.46 μg/ml | 5170 | Female | 22 | Day 28 | OD | 4 | 4 | 4 | 4 |
| CF-04 0.46 μg/ml | 5170 | Female | 22 | Baseline | OS | / | / | / | / |
| CF-04 0.46 μg/ml | 5170 | Female | 22 | Day 28 | OS | / | / | / | / |
| CF-04 0.46 μg/ml | 9258 | Female | 13 | Baseline | OD | 4 | 5 | 5 | 4.7 |
| CF-04 0.46 μg/ml | 9258 | Female | 13 | Day 28 | OD | 4 | 4 | 5 | 4.3 |
| CF-04 0.46 μg/ml | 9258 | Female | 13 | Baseline | OS | 4 | 6 | 5 | 5 |
| CF-04 0.46 μg/ml | 9258 | Female | 13 | Day 28 | OS | 4 | 3 | 5 | 4 |
| CF-04 2.3 μg/ml | 2400 | Female | 18 | Baseline | OD | 5 | 4 | 5 | 4.7 |
| CF-04 2.3 μg/ml | 2400 | Female | 18 | Day 28 | OD | 4 | 5 | 5 | 4.7 |
| CF-04 2.3 μg/ml | 2400 | Female | 18 | Baseline | OS | 4 | 5 | 4 | 4.3 |
| CF-04 2.3 μg/ml | 2400 | Female | 18 | Day 28 | OS | 5 | 4 | 6 | 5 |
| CF-04 2.3 μg/ml | 3701 | Male | 18 | Baseline | OD | 4 | 5 | 4 | 4.3 |
| CF-04 2.3 μg/ml | 3701 | Male | 18 | Day 28 | OD | 5 | 5 | 4 | 4.7 |
| CF-04 2.3 μg/ml | 3701 | Male | 18 | Baseline | OS | 4 | 4 | 5 | 4.3 |
| CF-04 2.3 μg/ml | 3701 | Male | 18 | Day 28 | OS | 5 | 6 | 7 | 6 |
| CF-04 2.3 μg/ml | 8291 | Male | 12 | Baseline | OD | 4 | 5 | 4 | 4.3 |
| CF-04 2.3 μg/ml | 8291 | Male | 12 | Day 28 | OD | 6 | 4 | 5 | 5 |
| CF-04 2.3 μg/ml | 8291 | Male | 12 | Baseline | OS | 4 | 4 | 5 | 4.3 |
| CF-04 2.3 μg/ml | 8291 | Male | 12 | Day 28 | OS | 5 | 5 | 5 | 5 |
| CF-04 2.3 μg/ml | 13053 | Male | 13 | Baseline | OD | 4 | 4 | 4 | 4 |
| CF-04 2.3 μg/ml | 13053 | Male | 13 | Day 28 | OD | 4 | 5 | 4 | 4.3 |
| CF-04 2.3 μg/ml | 13053 | Male | 13 | Baseline | OS | 5 | 5 | 4 | 4.7 |
| CF-04 2.3 μg/ml | 13053 | Male | 13 | Day 28 | OS | 5 | 4 | 5 | 4.7 |
| CF-04 4.6 μg/ml | 1822 | Female | 18 | Baseline | OD | 5 | 5 | 4 | 4.7 |
| CF-04 4.6 μg/ml | 1822 | Female | 18 | Day 28 | OD | 6 | 6 | 5 | 5.7 |
| CF-04 4.6 μg/ml | 1822 | Female | 18 | Baseline | OS | 5 | 5 | 4 | 4.7 |
| CF-04 4.6 μg/ml | 1822 | Female | 18 | Day 28 | OS | 5 | 5 | 5 | 5 |
| CF-04 4.6 μg/ml | 1927 | Male | 19 | Baseline | OD | 5 | 3 | 3 | 3.7 |
| CF-04 4.6 μg/ml | 1927 | Male | 19 | Day 28 | OD | 5 | 5 | 4 | 4.7 |
| CF-04 4.6 μg/ml | 1927 | Male | 19 | Baseline | OS | 5 | 5 | 5 | 5 |
| CF-04 4.6 μg/ml | 1927 | Male | 19 | Day 28 | OS | 5 | 5 | 6 | 5.3 |
| CF-04 4.6 μg/ml | 2870 | Female | 16 | Baseline | OD | 3 | 4 | 3 | 3.3 |
| CF-04 4.6 μg/ml | 2870 | Female | 16 | Day 28 | OD | 4 | 4 | 4 | 4 |
| CF-04 4.6 μg/ml | 2870 | Female | 16 | Baseline | OS | 4 | 5 | 5 | 4.7 |
| CF-04 4.6 μg/ml | 2870 | Female | 16 | Day 28 | OS | 5 | 4 | 5 | 4.7 |
| CF-04 4.6 μg/ml | 8033 | Male | 12 | Baseline | OD | 5 | 4 | 3 | 4 |
| CF-04 4.6 μg/ml | 8033 | Male | 12 | Day 28 | OD | 5 | 5 | 6 | 5.3 |
| CF-04 4.6 μg/ml | 8033 | Male | 12 | Baseline | OS | 3 | 4 | 4 | 3.7 |
| CF-04 4.6 μg/ml | 8033 | Male | 12 | Day 28 | OS | 5 | 4 | 4 | 4.3 |
| CF-04 50 μg/ml | 5 | Male | 24 | Baseline1 | OD | 3 | 5 | 4 | 4.0 |
| CF-04 50 μg/ml | 5 | Male | 24 | Baseline2 | OD | 5 | 4 | 4 | 4.3 |
| CF-04 50 μg/ml | 5 | Male | 24 | D 14 | OD | 3 | 5 | 3 | 3.7 |
| CF-04 50 μg/ml | 5 | Male | 24 | D 30 | OD | 4 | 5 | 4 | 4.3 |
| CF-04 50 μg/ml | 5 | Male | 24 | Baseline1 | OS | 5 | 4 | 4 | 4.3 |
| CF-04 50 μg/ml | 5 | Male | 24 | Baseline2 | OS | 4 | 5 | 5 | 4.7 |
| CF-04 50 μg/ml | 5 | Male | 24 | D 14 | OS | 4 | 6 | 3 | 4.3 |
| CF-04 50 μg/ml | 5 | Male | 24 | D 30 | OS | 5 | 4 | 4 | 4.3 |
| CF-04 50 μg/ml | 2870 | Female | 15 | Baseline1 | OD | 2 | 3 | 3 | 2.7 |
| CF-04 50 μg/ml | 2870 | Female | 15 | Baseline2 | OD | 2 | 2 | 2 | 2.0 |
| CF-04 50 μg/ml | 2870 | Female | 15 | D 14 | OD | 2 | 2 | 3 | 2.3 |
| CF-04 50 μg/ml | 2870 | Female | 15 | D 30 | OD | 2 | 2 | 2 | 2.0 |
| CF-04 50 μg/ml | 2870 | Female | 15 | Baseline1 | OS | 2 | 2 | 2 | 2.0 |
| CF-04 50 μg/ml | 2870 | Female | 15 | Baseline2 | OS | 2 | 2 | 2 | 2.0 |
| CF-04 50 μg/ml | 2870 | Female | 15 | D 14 | OS | 3 | 2 | 2 | 2.3 |
| CF-04 50 μg/ml | 2870 | Female | 15 | D 30 | OS | 2 | 3 | 2 | 2.3 |
| CF-04 50 μg/ml | 2930 | Female | 19 | Baseline1 | OD | 4 | 3 | 4 | 3.7 |
| CF-04 50 μg/ml | 2930 | Female | 19 | Baseline2 | OD | 3 | 3 | 4 | 3.3 |
| CF-04 50 μg/ml | 2930 | Female | 19 | D 14 | OD | 6 | 4 | 3 | 4.3 |
| CF-04 50 μg/ml | 2930 | Female | 19 | D 30 | OD | 3 | 3 | 3 | 3.0 |
| CF-04 50 μg/ml | 2930 | Female | 19 | Baseline1 | OS | 6 | 5 | 5 | 5.3 |
| CF-04 50 μg/ml | 2930 | Female | 19 | Baseline2 | OS | 5 | 5 | 7 | 5.7 |
| CF-04 50 μg/ml | 2930 | Female | 19 | D 14 | OS | 6 | 5 | 4 | 5.0 |
| CF-04 50 μg/ml | 2930 | Female | 19 | D 30 | OS | 5 | 6 | 6 | 5.7 |
| CF-04 50 μg/ml | 6707 | Male | 20 | Baseline1 | OD | 4 | 4 | 3 | 3.7 |
| CF-04 50 μg/ml | 6707 | Male | 20 | Baseline2 | OD | 4 | 3 | 4 | 3.7 |
| CF-04 50 μg/ml | 6707 | Male | 20 | D 14 | OD | 5 | 4 | 4 | 4.3 |
| CF-04 50 μg/ml | 6707 | Male | 20 | D 30 | OD | 5 | 5 | 4 | 4.7 |
| CF-04 50 μg/ml | 6707 | Male | 20 | Baseline1 | OS | 2 | 2 | 2 | 2.0 |
| CF-04 50 μg/ml | 6707 | Male | 20 | Baseline2 | OS | 3 | 4 | 4 | 3.7 |
| CF-04 50 μg/ml | 6707 | Male | 20 | D 14 | OS | 3 | 3 | 3 | 3.0 |
| CF-04 50 μg/ml | 6707 | Male | 20 | D 30 | OS | 5 | 4 | 5 | 4.7 |
| Restasis 0.05% | 1864 | Female | 20 | Baseline1 | OD | 2 | 3 | 2 | 2.3 |
| Restasis 0.05% | 1864 | Female | 20 | Baseline2 | OD | 2 | 2 | 2 | 2.0 |
| Restasis 0.05% | 1864 | Female | 20 | D 14 | OD | 3 | 3 | 2 | 2.7 |
| Restasis 0.05% | 1864 | Female | 20 | D 30 | OD | 4 | 3 | 3 | 3.3 |
| Restasis 0.05% | 1864 | Female | 20 | Baseline1 | OS | 2 | 2 | 2 | 2.0 |
| Restasis 0.05% | 1864 | Female | 20 | Baseline2 | OS | 1 | 2 | 2 | 1.7 |
| Restasis 0.05% | 1864 | Female | 20 | D 14 | OS | 3 | 2 | 2 | 2.3 |
| Restasis 0.05% | 1864 | Female | 20 | D 30 | OS | 2 | 3 | 3 | 2.7 |
| Restasis 0.05% | 5170 | Female | 21 | Baseline1 | OD | 3 | 2 | 2 | 2.3 |
| Restasis 0.05% | 5170 | Female | 21 | Baseline2 | OD | 2 | 2 | 2 | 2.0 |
| Restasis 0.05% | 5170 | Female | 21 | D 14 | OD | 3 | 4 | 3 | 3.3 |
| Restasis 0.05% | 5170 | Female | 21 | D 30 | OD | 4 | 5 | 5 | 4.7 |
| Restasis 0.05% | 5170 | Female | 21 | Baseline1 | OS | / | / | / | / |
| Restasis 0.05% | 5170 | Female | 21 | Baseline2 | OS | / | / | / | / |
| Restasis 0.05% | 5170 | Female | 21 | D 14 | OS | / | / | / | / |
| Restasis 0.05% | 5170 | Female | 21 | D 30 | OS | / | / | / | / |
| Restasis 0.05% | 6669 | Male | 23 | Baseline1 | OD | 3 | 3 | 4 | 3.3 |
| Restasis 0.05% | 6669 | Male | 23 | Baseline2 | OD | 4 | 3 | 5 | 4.0 |
| Restasis 0.05% | 6669 | Male | 23 | D 14 | OD | 5 | 4 | 5 | 4.7 |
| Restasis 0.05% | 6669 | Male | 23 | D 30 | OD | 5 | 5 | 6 | 5.3 |
| Restasis 0.05% | 6669 | Male | 23 | Baseline1 | OS | 5 | 4 | 4 | 4.3 |
| Restasis 0.05% | 6669 | Male | 23 | Baseline2 | OS | 4 | 5 | 4 | 4.3 |
| Restasis 0.05% | 6669 | Male | 23 | D 14 | OS | 5 | 5 | 4 | 4.7 |
| Restasis 0.05% | 6669 | Male | 23 | D 30 | OS | 3 | 5 | 4 | 4.0 |
| Restasis 0.05% | 7983 | Male | 12 | Baseline1 | OD | 4 | 5 | 4 | 4.3 |
| Restasis 0.05% | 7983 | Male | 12 | Baseline2 | OD | 5 | 4 | 4 | 4.3 |
| Restasis 0.05% | 7983 | Male | 12 | D 14 | OD | 5 | 5 | 3 | 4.3 |
| Restasis 0.05% | 7983 | Male | 12 | D 30 | OD | 4 | 5 | 4 | 4.3 |
| Restasis 0.05% | 7983 | Male | 12 | Baseline1 | OS | 4 | 5 | 5 | 4.7 |
| Restasis 0.05% | 7983 | Male | 12 | Baseline2 | OS | 5 | 4 | 5 | 4.7 |
| Restasis 0.05% | 7983 | Male | 12 | D 14 | OS | 4 | 5 | 4 | 4.3 |
| Restasis 0.05% | 7983 | Male | 12 | D 30 | OS | 4 | 5 | 5 | 4.7 |

The data of Table 4 was further analyzed by ANOVA, as shown in FIG. 4A-4B. As can be seen from FIG. 4A-4B, animals treated with 2.3 μg/ml CF-04 and 4.6 μg/ml CF-04 significantly prolonged the TBUT as compared to the placebo, comparative with the positive control Restasis, indicating effects of 2.3 μg/ml CF-04 and 4.6 μg/ml CF-04 in treatment of DE.

### Example 3. Effects of Exemplary Polypeptides of the Present Disclosure on Treatment of Acute DE

Effects of exemplary polypeptides of the present disclosure (SEQ ID NO. 29, CF-04) on treating DE were evaluated in Rhesus Macaque as described below. Briefly, healthy adult monkeys were placed in the dry eye chamber for 3 weeks to induce acute and severe dry eye symptoms through a dry environment. On day 21, CFS and TUBT were examined, and tear inflammatory factor IL-17a was assayed on day 23. Then animals with a single eye having a CFS score of 8-13 were enrolled in the study.

Enrolled animals were randomly divided into placebo group (saline, 3 monkeys, 6 eyes) and CF-04 group (5 μg/mL CF-04, 4 animals, 8 eyes). Both eyes of the animals were subject to treatment, 2 drops per eye, twice a day, CFS scores and TUBT were examined on day 7 and day 14 after the treatment, IL-17a was assayed on day 16 after the treatment.

### 1. Cornea Fluorescein Staining Score

CFS scores were firstly evaluated on baseline (day −2 of the treatment). The mean value of the CFS scores in control group and CF-04 group were 9.0±0.6 and 9.6±0.9, respectively. There was no significant difference between the mean CFS scores of the two groups (p=0.4719). After 7 days of treatment, mean CFS scores of animals in control group and CF-04 group were 8.7±1.4 and 5.9±1.8, respectively. The mean CFS of CF-04 group were significantly lower than control group (p=0.0194). After 14 days of treatment, mean CFS scores of animals in control group and CF-04 group were 9.0±1.3 and 7.0±2.2, respectively, with no significant difference. The CFS scores of each animal are as shown in Table 5 below,

TABLE 5
CFS scores of monkeys with the treatment of CF-04.

| | | | | CFS scores | | | | | | |
|---|---|---|---|---|---|---|---|---|---|---|
| Group | Monkey ID | Gender | Study Day | Eye | C | S | T | I | N | Total |
| Placebo | 7880 | Female | Baseline | OD | 2 | 2 | 1 | 2 | 2 | 9 |
| Placebo | 7880 | Female | Day 7 | OD | 1 | 1 | 1 | 2 | 2 | 7 |
| Placebo | 7880 | Female | Day 14 | OD | 1 | 1 | 1 | 2 | 2 | 7 |
| Placebo | 7880 | Female | Baseline | OS | 1 | 2 | 2 | 2 | 1 | 8 |
| Placebo | 7880 | Female | Day 7 | OS | 1 | 2 | 1 | 2 | 1 | 8 |
| Placebo | 7880 | Female | Day 14 | OS | 1 | 2 | 2 | 2 | 2 | 9 |
| Placebo | 9210 | Female | Baseline | OD | 1 | 2 | 2 | 2 | 2 | 9 |
| Placebo | 9210 | Female | Day 7 | OD | 1 | 2 | 2 | 2 | 2 | 9 |
| Placebo | 9210 | Female | Day 14 | OD | 1 | 2 | 2 | 2 | 2 | 9 |
| Placebo | 9210 | Female | Baseline | OS | 1 | 1 | 2 | 3 | 3 | 10 |
| Placebo | 9210 | Female | Day 7 | OS | 2 | 2 | 2 | 2 | 3 | 11 |
| Placebo | 9210 | Female | Day 14 | OS | 2 | 2 | 2 | 2 | 3 | 11 |
| Placebo | 9464 | Female | Baseline | OD | 1 | 1 | 3 | 2 | 2 | 9 |
| Placebo | 9464 | Female | Day 7 | OD | 1 | 1 | 1 | 3 | 2 | 8 |
| Placebo | 9464 | Female | Day 14 | OD | 1 | 2 | 2 | 2 | 2 | 9 |
| Placebo | 9464 | Female | Baseline | OS | 1 | 1 | 2 | 2 | 3 | 9 |
| Placebo | 9464 | Female | Day 7 | OS | 1 | 3 | 2 | 1 | 2 | 9 |
| Placebo | 9464 | Female | Day 14 | OS | 1 | 2 | 1 | 2 | 3 | 9 |
| CF-04 5 μg/ml | 7300 | Female | Baseline | OD | 2 | 1 | 3 | 1 | 2 | 9 |
| CF-04 5 μg/ml | 7300 | Female | Day 7 | OD | 1 | 1 | 1 | 1 | 2 | 6 |
| CF-04 5 μg/ml | 7300 | Female | Day 14 | OD | 1 | 2 | 1 | 1 | 2 | 7 |
| CF-04 5 μg/ml | 7300 | Female | Baseline | OS | 1 | 2 | 1 | 2 | 2 | 8 |
| CF-04 5 μg/ml | 7300 | Female | Day 7 | OS | 1 | 1 | 0 | 1 | 2 | 5 |
| CF-04 5 μg/ml | 7300 | Female | Day 14 | OS | 1 | 2 | 1 | 1 | 1 | 6 |
| CF-04 5 μg/ml | 7736 | Female | Baseline | OD | 2 | 2 | 2 | 2 | 2 | 10 |
| CF-04 5 μg/ml | 7736 | Female | Day 7 | OD | 2 | 1 | 1 | 2 | 1 | 7 |
| CF-04 5 μg/ml | 7736 | Female | Day 14 | OD | 2 | 2 | 2 | 2 | 2 | 10 |
| CF-04 5 μg/ml | 7736 | Female | Baseline | OS | 2 | 2 | 2 | 3 | 2 | 11 |
| CF-04 5 μg/ml | 7736 | Female | Day 7 | OS | 2 | 1 | 2 | 1 | 2 | 8 |
| CF-04 5 μg/ml | 7736 | Female | Day 14 | OS | 2 | 2 | 2 | 1 | 2 | 9 |
| CF-04 5 μg/ml | 8246 | Female | Baseline | OD | 2 | 2 | 2 | 2 | 2 | 10 |
| CF-04 5 μg/ml | 8246 | Female | Day 7 | OD | 1 | 0 | 0 | 1 | 2 | 4 |
| CF-04 5 μg/ml | 8246 | Female | Day 14 | OD | 2 | 2 | 1 | 1 | 2 | 8 |
| CF-04 5 μg/ml | 8246 | Female | Baseline | OS | 2 | 2 | 2 | 2 | 2 | 10 |
| CF-04 5 μg/ml | 8246 | Female | Day 7 | OS | 1 | 2 | 1 | 2 | 2 | 8 |
| CF-04 5 μg/ml | 8246 | Female | Day 14 | OS | 1 | 2 | 1 | 2 | 2 | 8 |
| CF-04 5 μg/ml | 9742 | Female | Baseline | OD | 2 | 2 | 2 | 2 | 2 | 10 |
| CF-04 5 μg/ml | 9742 | Female | Day 7 | OD | 0 | 1 | 0 | 1 | 1 | 3 |
| CF-04 5 μg/ml | 9742 | Female | Day 14 | OD | 1 | 1 | 0 | 1 | 1 | 4 |
| CF-04 5 μg/ml | 9742 | Female | Baseline | OS | 1 | 2 | 2 | 2 | 2 | 9 |
| CF-04 5 μg/ml | 9742 | Female | Day 7 | OS | 1 | 1 | 1 | 1 | 2 | 6 |
| CF-04 5 μg/ml | 9742 | Female | Day 14 | OS | 0 | 1 | 0 | 1 | 2 | 4 |

### 2. Tear Break-Up Time (TBUT)

TUBT were firstly evaluated on baseline (day-2 of the treatment). The mean TUBT in control group and CF-04 group were 4.0±0.8s and 3.8±0.6s, respectively. There was no significant difference between the mean TUBT of the two groups (p>0.9999). After 7 days of treatment, mean TUBT of animals in control group and CF-04 group were 3.5±0.6s and 4.9±0.6s, respectively. The mean TUBT of CF-04 group were significantly prolonged than control group (p=0.0042). After 14 days of treatment, mean TUBT of animals in control group and CF-04 group were 3.6±0.5s and 4.6±0.9s, respectively, with no significant difference. The TUBT of each animal is as shown in Table 6 below,

TABLE 6
TBUT of animals treated with CF-04

| | | | | Tear Break-up Time (s) | | | | |
|---|---|---|---|---|---|---|---|---|
| Group | Monkey ID | Gender | Study Day | Eye | 1st | 2nd | 3rd | Mean |
| Placebo | 7880 | Female | Baseline | OD | 5 | 4 | 5 | 4.7 |
| Placebo | 7880 | Female | Day 7 | OD | 3 | 4 | 3 | 3.3 |
| Placebo | 7880 | Female | Day 14 | OD | 3 | 3 | 3 | 3.0 |
| Placebo | 7880 | Female | Baseline | OS | 3 | 5 | 3 | 3.7 |
| Placebo | 7880 | Female | Day 7 | OS | 2 | 5 | 4 | 3.7 |
| Placebo | 7880 | Female | Day 14 | OS | 3 | 4 | 4 | 3.7 |
| Placebo | 9210 | Female | Baseline | OD | 5 | 4 | 5 | 4.7 |
| Placebo | 9210 | Female | Day 7 | OD | 2 | 4 | 3 | 3.0 |
| Placebo | 9210 | Female | Day 14 | OD | 4 | 4 | 4 | 4.0 |
| Placebo | 9210 | Female | Baseline | OS | 4 | 4 | 4 | 4.0 |
| Placebo | 9210 | Female | Day 7 | OS | 2 | 3 | 3 | 2.7 |
| Placebo | 9210 | Female | Day 14 | OS | 3 | 3 | 3 | 3.0 |
| Placebo | 9464 | Female | Baseline | OD | 2 | 3 | 3 | 2.7 |
| Placebo | 9464 | Female | Day 7 | OD | 4 | 3 | 5 | 4.0 |
| Placebo | 9464 | Female | Day 14 | OD | 4 | 4 | 4 | 4.0 |
| Placebo | 9464 | Female | Baseline | OS | 4 | 4 | 5 | 4.3 |
| Placebo | 9464 | Female | Day 7 | OS | 4 | 4 | 5 | 4.3 |
| Placebo | 9464 | Female | Day 14 | OS | 4 | 3 | 5 | 4.0 |
| CF-04 5 μg/ml | 7300 | Female | Baseline | OD | 3 | 2 | 3 | 2.7 |
| CF-04 5 μg/ml | 7300 | Female | Day 7 | OD | 4 | 5 | 5 | 4.7 |
| CF-04 5 μg/ml | 7300 | Female | Day 14 | OD | 4 | 4 | 3 | 3.7 |
| CF-04 5 μg/ml | 7300 | Female | Baseline | OS | 4 | 4 | 3 | 3.7 |
| CF-04 5 μg/ml | 7300 | Female | Day 7 | OS | 6 | 5 | 4 | 5.0 |
| CF-04 5 μg/ml | 7300 | Female | Day 14 | OS | 4 | 4 | 6 | 4.7 |
| CF-04 5 μg/ml | 7736 | Female | Baseline | OD | 3 | 3 | 3 | 3.0 |
| CF-04 5 μg/ml | 7736 | Female | Day 7 | OD | 3 | 4 | 5 | 4.0 |
| CF-04 5 μg/ml | 7736 | Female | Day 14 | OD | 3 | 3 | 3 | 3.0 |
| CF-04 5 μg/ml | 7736 | Female | Baseline | OS | 5 | 3 | 5 | 4.3 |
| CF-04 5 μg/ml | 7736 | Female | Day 7 | OS | 6 | 5 | 5 | 5.3 |
| CF-04 5 μg/ml | 7736 | Female | Day 14 | OS | 4 | 3 | 5 | 4.0 |
| CF-04 5 μg/ml | 8246 | Female | Baseline | OD | 5 | 5 | 3 | 4.3 |
| CF-04 5 μg/ml | 8246 | Female | Day 7 | OD | 5 | 4 | 5 | 4.7 |
| CF-04 5 μg/ml | 8246 | Female | Day 14 | OD | 5 | 5 | 6 | 5.3 |
| CF-04 5 μg/ml | 8246 | Female | Baseline | OS | 3 | 4 | 5 | 4.0 |
| CF-04 5 μg/ml | 8246 | Female | Day 7 | OS | 4 | 4 | 5 | 4.3 |
| CF-04 5 μg/ml | 8246 | Female | Day 14 | OS | 5 | 5 | 5 | 5.0 |
| CF-04 5 μg/ml | 9742 | Female | Baseline | OD | 4 | 4 | 4 | 4.0 |
| CF-04 5 μg/ml | 9742 | Female | Day 7 | OD | 6 | 6 | 6 | 6.0 |
| CF-04 5 μg/ml | 9742 | Female | Day 14 | OD | 6 | 5 | 5 | 5.3 |
| CF-04 5 μg/ml | 9742 | Female | Baseline | OS | 4 | 4 | 4 | 4.0 |
| CF-04 5 μg/ml | 9742 | Female | Day 7 | OS | 5 | 6 | 5 | 5.3 |
| CF-04 5 μg/ml | 9742 | Female | Day 14 | OS | 7 | 5 | 5 | 5.7 |

### Example 4. Effects of Exemplary Polypeptides of the Present Disclosure on Inhibiting Thermal Aggregation of BSA

Exemplary polypeptides of the present application as shown in Table 7 below were dissolved in PBS and serially diluted for 3 times. Then 150 μL polypeptides were mixed with 150 μL 200 μM BSA and the mixture was incubated in 98° C. water bath for 30 minutes, 200 μL mixture was added to the detection plate and absorbance was assayed at 600 nM wavelength. Inhibition rate was calculated as:

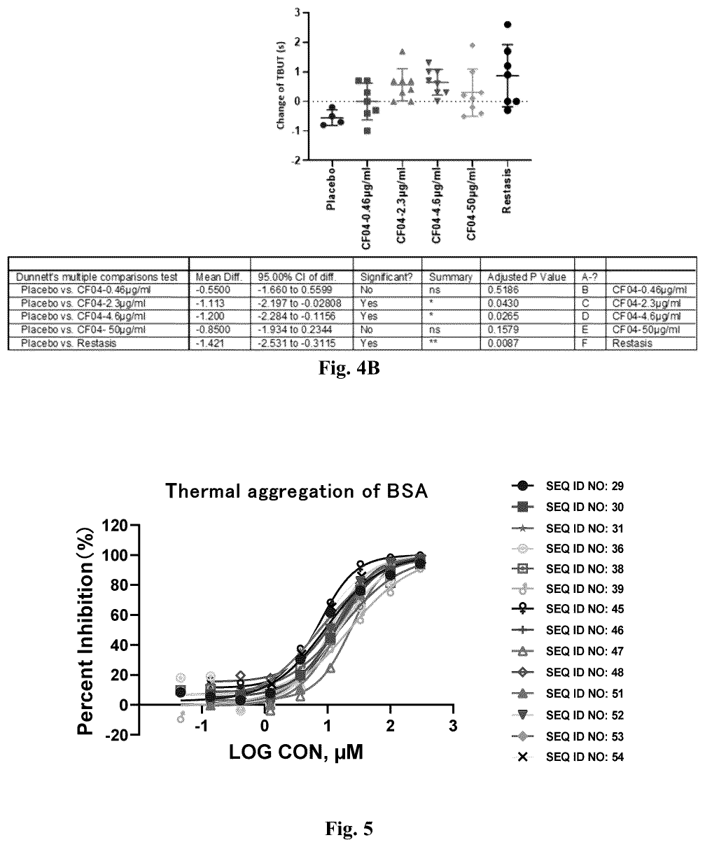

100−[(ODₛₐₘₚₗₑ−OD_{PBS})/(OD_{BSA}−−OD_{PBS})]*100

wherein OD_{BSA} is absorbance of sample containing only BSA, OD_{PBS} is absorbance of sample containing PBS only, IC₅₀ was calculated and analyzed by Graphpad by using vs log concentration. The result is as shown in FIG. 5 and Table 7 below,

TABLE 7
Effects of exemplary polypeptides of the present disclosure
on inhibiting thermal aggregation of BSA

| | IC50 (μM) for inhibiting thermal |
|---|---|
| Sample | aggregation of BSA |
| CF-04 (SEQ ID NO: 29) | ~10.15 |
| CF-10 (SEQ ID NO: 30) | 13.76 |
| CF-11 (SEQ ID NO: 31) | ~7 |
| CF-22 (SEQ ID NO: 36) | 16.94 |
| CF-24 (SEQ ID NO: 38) | 17.56 |
| CF-25 (SEQ ID NO: 39) | 21.54 |
| CF-28 (SEQ ID NO: 45) | 7.48 |
| CF-29 (SEQ ID NO: 46) | 13.84 |
| CF-30 (SEQ ID NO: 47) | 21.31 |
| CF-31 (SEQ ID NO: 48) | 12.84 |
| CF-38 (SEQ ID NO: 51) | 13.76 |
| CF-39 (SEQ ID NO: 52) | 10.54 |
| CF-40 (SEQ ID NO: 53) | 11.97 |
| CF-43 (SEQ ID NO: 54) | 6.77 |

### Example 5. Effects of the Polypeptides of the Present Disclosure on Cellular ROS Level Induced by DAMP

90 μL HUVEC cells were seeded into 96-well plates at 10000/well overnight, 1 h before detection, ROS reagent (Thermo, Cat #88-5930-74) was added to cell culture plates as working solution, 90 μl/well. Meanwhile, exemplary polypeptides of the present application as shown in Table 8 below were serially diluted for 3 times by cell culture medium, 60 μL polypeptides at different concentrations were mixed with 60 μL 400 μg/ml oxHSA. Then 20 μl mixture of polypeptides and oxHSA were transferred to cell cultures and incubated with cells for 40 minutes. Fluorescence was detected at 490 nm/525 nm excitation/emission wavelength. Inhibition rate was calculated as:

$$100 - [ ( {L}_{sample} - {L}_{PBS / medium} ) / ( {L}_{oxHSA} - {L}_{PBS / medium} ) ] * 100$$

The result is as shown in Table 8 below,

TABLE 8
Effects of the polypeptides of the present disclosure
on cellular ROS level induced by DAMP

| | IC50 (μM) for cellular ROS level induced |
|---|---|
| Sample | by DAMP |
| CF-38 (SEQ ID NO: 51) | 4.12 |
| CF-39 (SEQ ID NO: 52) | 3.91 |
| CF-40 (SEQ ID NO: 53) | 5.87 |
| CF-43 (SEQ ID NO: 54) | 6.13 |

### Example 6. Effects of the Polypeptides of the Present Disclosure on Inhibiting oxLDL Uptake

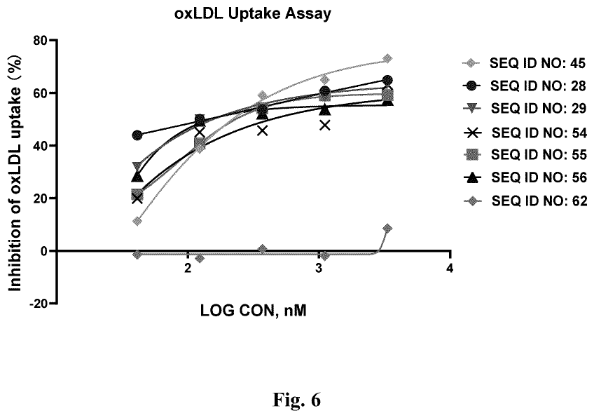

THP-1 cells (ATCC, 30-2001) were resuspended and counted in RPMI-1640 complete medium (10% fetal bovine serum, 1% bispecific antibody, 0.05 mM-mercaptoethanol) containing 100 nM PMA (Sigma-Aldrich, P1585). Then 30000 cells were seeded in each well of 96-well plates and cultured at 37° C. for 72 hours. Then the medium was replaced with RPMI-1640 complete medium without PMA and cells were cultured for another 48 h to obtain THP-1 induced differentiated macrophages. Exemplary polypeptides of the present application were serially diluted by RPMI-1640 complete medium. Then each of the polypeptides was mixed with oxLDL-Dylight488 solution (Cayman Chemical, 601180). The medium of the cell culture was replaced by the polypeptide and oxLDL-Dylight488 mixture, and the cells were cultured at 37° C. for 5-6 hours. After the incubation, cells in each well were rinsed and washed by PBS, resuspended in 200 μL PBS supplemented with 7-AAD, collected into FACS tube via filter and analyzed by flow cytometry (BD, FACSCelesta). Geometric mean fluorescence intensity (gMFI) of 7-AAD cells was analyzed in Dylight488 channel, oxLDL uptake rate was calculated as below:

$$oxLDL uptake rate = ( gMFI of experimental group - gMFI of negative control group ) / ( gMFI of positive conrtol group - MeanFI of negative control group ) \times 100 \%$$

where the positive group contains oxLDL only, and the negative group does not contain oxLDL or the polypeptide of the present application. The result is as shown in FIG. 6.

### Example 7. Binding Capability of Exemplary Polypeptides of the Present Disclosure with Oxidized Protein

1 mg/ml HSA protein was oxidized with 8 mM NaClO in the dark for 30 min. The reactant was added to a 3KD dialysis bag and dialyzed in PBS at 4° C. for 24 hours, during which the fluid was replaced with fresh dialysate 2 to 3 times. After filter sterilization, protein was quantified using BCA method. The oxidized protein was mixed with the target protein at a mass ratio of 1:2, and incubated at 4° C. for 24 hours. After the reaction, samples were subject to SDS-Page electrophoresis, and pictures were taken after Coomassie brilliant blue staining.

TABLE 9
Binding capability of exemplary polypeptides of
the present disclosure with oxidized protein.

| Polypeptides | Binding capability with oxidized protein (Y/N) |
|---|---|
| CF-01 (SEQ ID NO: 28) | Y |
| CF-04 (SEQ ID NO: 29) | Y |
| CF-11 (SEQ ID NO: 31) | Y |
| CF-18 (SEQ ID NO: 32) | Y |
| CF-19 (SEQ ID NO: 33) | Y |
| CF-20 (SEQ ID NO: 34) | Y |
| CF-23 (SEQ ID NO: 37) | Y |
| CF-24 (SEQ ID NO: 38) | Y |
| CF-25 (SEQ ID NO: 39) | Y |
| CF-26 (SEQ ID NO: 40) | Y |
| CF-27 (SEQ ID NO: 41) | Y |
| CF-28 (SEQ ID NO: 45) | Y |
| CF-29 (SEQ ID NO: 46) | Y |
| CF-30 (SEQ ID NO: 47) | Y |
| CF-31 (SEQ ID NO: 48) | Y |

### Example 8. Cell Binding Capability of the Polypeptides of the Present Disclosure

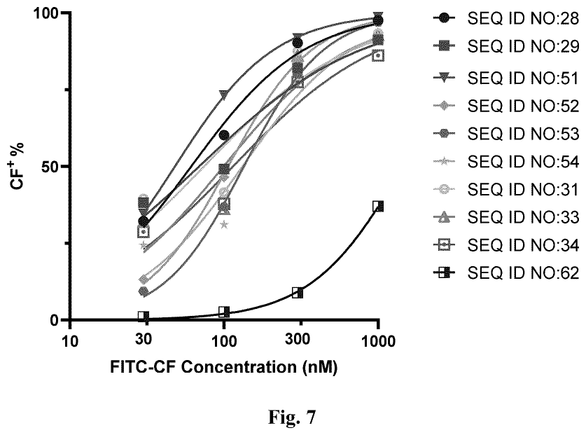

Peripheral blood mononuclear cells (PBMCs, SAILY BIO) were collected at room temperature by 400 g centrifuge, and the resuspended and counted in RPMI-1640 complete medium (10% fetal bovine serum, 1% bispecific antibody, 0.05 mM β-mercaptoethanol), 1×10⁶ cells were seeded in each well of 96-well plates and each were supplemented with FITC labelled CF polypeptides of the present application (Sigma-Aldrich, FITC1). The concentrations of the FITC labelled CF polypeptides in cell cultures were 0 nM, 30 nM, 100 nM, 300 nM, 1000 nM, respectively. Then the cells were cultured at 37° C. for 30 minutes. After incubation, cells in each well were washed by PBS and resuspended in 100 μL PBS, supplemented with BV421-CD16 (BD, 3G8) and BV605-CD14 (BD, M5E2) fluorescent antibodies. Then the cells were incubated at 4° C. in dark for 30 minutes. After incubation, cells were washed by PBS and resuspended in 200 μL PBS, collected into FACS tube via filter and analyzed by flow cytometry (BD, FACSCelesta). Fluorescence signal of CD16⁻CD14⁺ monocytes in each PBMC sample was analyzed in FITC channel. The ratio of CD16⁻CD14⁺CF⁺ monocytes in the sample was calculated, as shown in FIG. 7.

### Example 9. Pharmacodynamics Test of the Polypeptides on Dry Eyes in Mice Induced by Low Humidity Environment and Scopolamine Hydrobromide

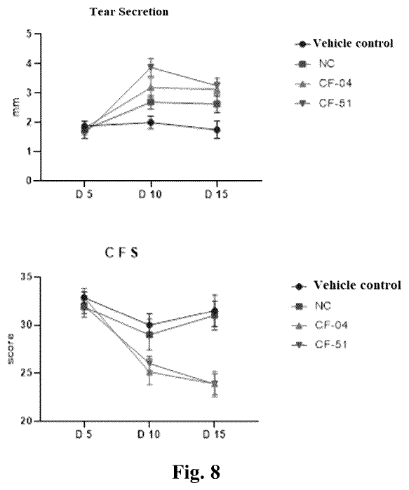

Thirty two mice (C57BL/6JShjh) of similar tear secretion and corneal fluorescein sodium scores were divided into 4 groups (n=8), negative control, vehicle control, treatment group 1 and treatment group 2. All animals were subject to a humidity of 10% to 30%, and were given 0.3 mL Scopolamine Hydrobromide (Sigma-Aldrich, 0.75 mg/mL) subcutaneously to induce dry eye, twice a day from day 1 (D1) to day 15 (D15). Animals from negative group were not treated, while the other groups were treated with vehicle only, the polypeptide of SEQ ID NO: 29, and the artificial polypeptide of SEQ ID NO: 75 respectively at a dose of 3 mL/eye twice a day (separated by around 8 hrs), from D6 to D15. Tear secretion was measured at D-1, D5, D10, and D15 with phenol red thread test for all animals and the tear secretion is measured as the length (mm) of wetted thread (FIG. 8, upper diagram).

Corneal sodium fluorescein scores were evaluated for all animals on D-1, D5, D10 and D15 according to the following protocol. After dripping the fluorescein sodium solution (1.5 μL, 0.5%) into the upper conjunctival sac of the animals, 1.25 mL of sterile saline was used to wash the animal's conjunctival sac every 10 s for 3 consecutive times. After dyeing for about 5 minutes, a slit lamp (+cobalt blue filter) was used to observe the ocular surface, pictures were taken, and on-site scoring were performed. Improved NEI scale for grading fluorescein staining was used to score the cornea of each animal. The cornea was divided into 5 regions (1—central area, 2—upper, 3—temporal, 4—nasal, 5—lower), each region was given a highest score of 8, and a lowest score of 0 for no staining at all (Grade 1: the stained area is 1% to 25% of the corresponding region; Grade 2: the stained area is 26%˜50% of the corresponding region; Grade 3: the stained area is 51%˜75% of the corresponding region; Grade 4: the stained area is 76%˜100% of the corresponding region). For densely/confluent staining, additional points were given according to the area of such densely/confluent stained region (1: 1%˜25%; 2: 26%˜50%; 3: 51%˜75% and 4: 76%˜100%). The maximum total score for each eye is 40 points. The total score of sodium fluorescein staining was calculated for each eye of all the animals (FIG. 8, lower diagram).

While preferred embodiments of the present invention have been shown and described herein, it will be obvious to those skilled in the art that such embodiments are provided by way of example only. It is not intended that the invention be limited by the specific examples provided within the specification. While the invention has been described with reference to the aforementioned specification, the descriptions and illustrations of the embodiments herein are not meant to be construed in a limiting sense. Numerous variations, changes, and substitutions will now occur to those skilled in the art without departing from the invention. Furthermore, it shall be understood that all aspects of the invention are not limited to the specific depictions, configurations or relative proportions set forth herein which depend upon a variety of conditions and variables. It should be understood that various alternatives to the embodiments of the invention described herein may be employed in practicing the invention. It is therefore contemplated that the invention shall also cover any such alternatives, modifications, variations or equivalents. It is intended that the following claims define the scope of the invention and that methods and structures within the scope of these claims and their equivalents be covered thereby.

SEQUENCE LISTING

| SEQ | |
|---|---|
| ID | |
| NO | Sequences |
| 1 | SEKKQPVDLGLLEEDDEFEEFPAEDWAGLDEDEDAHVWEDNWDDDNVEDDFSNQLRA |
| 2 | TVLLMILVCETPYGCYVLHQKGRMCSAFLCC |
| 3 | TVLLMILVAETPYGAYVLHQKGRMASAFLAA |
| 4 | TVLLMILVCETPYGCYVLHQKGRMCSAFLCA |
| 5 | TVLLMILVVETPYGVYVLHQKGRMVSAFLVV |
| 6 | TVLLMILVAETPYGAYVLHQKGRMVSAFLVV |
| 7 | TVLLMILVAETPYGVYVLHQKGRMASAFLVV |
| 8 | TVLLMILVAETPYGVYVLHQKGRMVSAFLAV |
| 9 | VLLMILVAETPYGVYVLHQKGRMVSAFLVA |
| 10 | TVLLMILVVETPYGAYVLHQKGRMASAFLVV |
| 11 | TVLLMILVVETPYGAYVLHQKGRMVSAFLAV |
| 12 | TVLLMILVVETPYGAYVLHQKGRMVSAFLVA |
| 13 | TVLLMILVVETPYGVYVLHQKGRMASAFLAV |
| 14 | TVLLMILVVETPYGVYVLHQKGRMASAFLVA |
| 15 | TVLLMILVVETPYGVYVLHQKGRMVSAFLAA |
| 16 | TVLLVILVAETPYGAYVLHQKGRVASAFLAA |
| 17 | GLCALCLMVRQPCFCLEIYMGVKHTCLYVST |
| 18 | SLLCVFPTIECCRYKMGLLYHCVACTQGMLV |
| 19 | SEFEDANKEQGEVPNDDVSDELPKLLDDDEDELRGALFFQWHDWEEAEDNDWDVEDA |
| 20 | SELDFWDNWKDRQADLPAASDDEDFENDLAEVDHEDPLVDGNEWDGDEVEFELEEQK |
| 21 | SDADEFDDDNSVWGWEDDEVEPNDAFQPLGDVRANEELDEKDHKLDEEADLQWEFEL |
| 22 | SQWAPLDHLESDEWGAFEDEDENNGDVEELDPEERDDEDLFQKDVLVAKADDEEWDN |
| 23 | STKKQPVDLGLLETDSEFTEFPATSWAGLSESESAHVWTSNWDSSNVTSDFSNQLRA |
| 24 | STKKQPVDLGLLETDDEFEEFPATSWAGLSEDESAHVWESNWDDSNVTSDFSNQLRA |
| 25 | STKKQPVDLGLLEEDDEFEEFPAESWAGLDEDESAHVWEDNWDDDNVTSDFSNQLRA |
| 26 | SEKKQPVDLGLLEEDDEFEEFPAEDWAGLDEDESAHVWEDNWDDDNVTSDFSNQLRA |
| 27 | SEKKQPVDLGLLEEDDEFEEFPAEDWAGLDEDEDAHVWEDNWDDDNVTSDFSNQLRA |
| 28 | SEKKQPVDLGLLEEDDEFEEFPAEDWAGLDEDEDAHVWEDNWDDDNVEDDFSNQLRAT |
| | VLLMILVCETPYGCYVLHQKGRMCSAFLCC |
| 29 | SEKKQPVDLGLLEEDDEFEEFPAEDWAGLDEDEDAHVWEDNWDDDNVEDDFSNQLRAT |
| | VLLMILVAETPYGAYVLHQKGRMASAFLAA |
| 30 | SEKKQPVDLGLLEEDDEFEEFPAEDWAGLDEDEDAHVWEDNWDDDNVEDDFSNQLRAT |
| | VLLMILVCETPYGCYVLHQKGRMCSAFLCA |
| 31 | SEKKQPVDLGLLEEDDEFEEFPAEDWAGLDEDEDAHVWEDNWDDDNVEDDFSNQLRAT |
| | VLLMILVVETPYGVYVLHQKGRMVSAFLVV |
| 32 | SEKKQPVDLGLLEEDDEFEEFPAEDWAGLDEDEDAHVWEDNWDDDNVEDDFSNQLRAT |
| | VLLMILVAETPYGAYVLHQKGRMVSAFLVV |
| 33 | SEKKQPVDLGLLEEDDEFEEFPAEDWAGLDEDEDAHVWEDNWDDDNVEDDFSNQLRAT |
| | VLLMILVAETPYGVYVLHQKGRMASAFLVV |
| 34 | SEKKQPVDLGLLEEDDEFEEFPAEDWAGLDEDEDAHVWEDNWDDDNVEDDFSNQLRAT |
| | VLLMILVAETPYGVYVLHQKGRMVSAFLAV |
| 35 | SEKKQPVDLGLLEEDDEFEEFPAEDWAGLDEDEDAHVWEDNWDDDNVEDDFSNQLRAT |
| | VLLMILVAETPYGVYVLHQKGRMVSAFLVA |
| 36 | SEKKQPVDLGLLEEDDEFEEFPAEDWAGLDEDEDAHVWEDNWDDDNVEDDFSNQLRAT |
| | VLLMILVVETPYGAYVLHQKGRMASAFLVV |
| 37 | SEKKQPVDLGLLEEDDEFEEFPAEDWAGLDEDEDAHVWEDNWDDDNVEDDFSNQLRAT |
| | VLLMILVVETPYGAYVLHQKGRMVSAFLAV |
| 38 | SEKKQPVDLGLLEEDDEFEEFPAEDWAGLDEDEDAHVWEDNWDDDNVEDDFSNQLRAT |
| | VLLMILVVETPYGAYVLHQKGRMVSAFLVA |
| 39 | SEKKQPVDLGLLEEDDEFEEFPAEDWAGLDEDEDAHVWEDNWDDDNVEDDFSNQLRAT |
| | VLLMILVVETPYGVYVLHQKGRMASAFLAV |
| 40 | SEKKQPVDLGLLEEDDEFEEFPAEDWAGLDEDEDAHVWEDNWDDDNVEDDFSNQLRAT |
| | VLLMILVVETPYGVYVLHQKGRMASAFLVA |
| 41 | SEKKQPVDLGLLEEDDEFEEFPAEDWAGLDEDEDAHVWEDNWDDDNVEDDFSNQLRAT |
| | VLLMILVVETPYGVYVLHQKGRMVSAFLAA |
| 42 | SEKKQPVDLGLLEEDDEFEEFPAEDWAGLDEDEDAHVWEDNWDDDNVEDDFSNQLRAG |
| | LCALCLMVRQPCFCLEIYMGVKHTCLYVST |
| 43 | SEKKQPVDLGLLEEDDEFEEFPAEDWAGLDEDEDAHVWEDNWDDDNVEDDFSNQLRAS |
| | LLCVFPTIECCRYKMGLLYHCVACTQGMLV |
| 44 | SEKKQPVDLGLLEEDDEFEEFPAEDWAGLDEDEDAHVWEDNWDDDNVEDDFSNQLRAT |
| | VLLVILVAETPYGAYVLHQKGRVASAFLAA |
| 45 | SEFEDANKEQGEVPNDDVSDELPKLLDDDEDELRGALFFQWHDWEEAEDNDWDVEDAT |
| | VLLMILVCETPYGCYVLHQKGRMCSAFLCC |
| 46 | SELDFWDNWKDRQADLPAASDDEDFENDLAEVDHEDPLVDGNEWDGDEVEFELEEQKT |
| | VLLMILVCETPYGCYVLHQKGRMCSAFLCC |
| 47 | SDADEFDDDNSVWGWEDDEVEPNDAFQPLGDVRANEELDEKDHKLDEEADLQWEFELT |
| | VLLMILVCETPYGCYVLHQKGRMCSAFLCC |
| 48 | SQWAPLDHLESDEWGAFEDEDFNNGDVEELDPEERDDEDLFQKDVLVAKADDEEWDNT |
| | VLLMILVCETPYGCYVLHQKGRMCSAFLCC |
| 49 | STKKQPVDLGLLETDSEFTEFPATSWAGLSESESAHVWTSNWDSSNVTSDFSNQLRATVL |
| | LMILVCETPYGCYVLHQKGRMCSAFLCC |
| 50 | STKKQPVDLGLLETDDEFEEFPATSWAGLSEDESAHVWESNWDDSNVTSDFSNQLRATVL |
| | LMILVCETPYGCYVLHQKGRMCSAFLCC |
| 51 | STKKQPVDLGLLEEDDEFEEFPAESWAGLDEDESAHVWEDNWDDDNVTSDFSNQLRATV |
| | LLMILVCETPYGCYVLHQKGRMCSAFLCC |
| 52 | SEKKQPVDLGLLEEDDEFEEFPAEDWAGLDEDESAHVWEDNWDDDNVTSDFSNQLRAT |
| | VLLMILVCETPYGCYVLHQKGRMCSAFLCC |
| 53 | SEKKQPVDLGLLEEDDEFEEFPAEDWAGLDEDEDAHVWEDNWDDDNVTSDFSNQLRAT |
| | VLLMILVCETPYGCYVLHQKGRMCSAFLCC |
| 54 | SEKKQPVDLGLLEEDDEFEEFPAEDWAGLDEDEDAHVWEDNWDDDNVTSDFSNQLRAT |
| | VLLMILVCETPYGCYVLHQKGRMCS |
| 55 | SEKKQPVDLGLLEEDDEFEEFPAEDWAGLDEDEDAHVWEDNWDDDNVTSDFSNQLRAT |
| | VLLMILVCETPYGCYVLHQK |
| 56 | SEKKQPVDLGLLEEDDEFEEFPAEDWAGLDEDEDAHVWEDNWDDDNVTSDFSNQLRAT |
| | VLLMILVCET |
| 57 | SEKKQPVDLGLLEEDDEFEEFPAEDWAGLDEDEDAHVWEDNWDDDNVTSDFSNQLRAT |
| | VLLMI |
| 58 | SEKKQPVDLGLLEEDDEFEEFPAEDWAGLDEDEDAHVWEDNWDDDNVTSDFSNQLRA |
| 59 | SEKKQPVDLGLLEEDDEFEEFPAEDWAGLDEDEDAHVWEDNWDDDNVTSDFSNQLRA |
| 60 | SEKKQPVDLGLLEEDDEFEEFPAEDWAGLDEDEDAHVWEDNWDDDNVTSDFSNQLRA |
| 61 | SEKKQPVDLGLLEEDDEFEEFPAEDWAGLDEDEDAHVWEDNWDDDNVTSDFSNQLRA |
| 62 | SEKKQPVDLGLLEEDDEFEEFPAEDWAGLDEDEDAHVWEDNWDDDNVEDDFSNQLRAE |
| | LEKHGYKMETS |
| 63 | MSEKKQPVDLGLLEEDDEFEEFPAEDWAGLDEDEDAHVWEDNWDDDNVEDDFSNQLRA |
| | TVLLMILVCETPYGCYVLHQKGRMCSAFLCC |
| 64 | MSEKKQPVDLGLLEEDDEFEEFPAEDWAGLDEDEDAHVWEDNWDDDNVEDDFSNQLRA |
| | TVLLMILVCETPYGCYVLHQKGRMCSAFLCC |
| 65 | MSEKKQPVDLGLLEEDDEFEEFPAEDWAGLDEDEDAHVWEDNWDDDNVEDDFSNQLRV |
| | TVLLMILVCETLYGCYVLHQKGRMCSAFLCC |
| 66 | MSEKKQPVDLGLLEEDDEFEEFPAEDWAGLDEDEDAHVWEDNWDDDNVEDDFSNQLRA |
| | TILLMILVCETPYGCYVLHQKGRMCSAFLCC |
| 67 | MSEKKQPVDLGLLEEDDEFEEFPAEDWAGLDEDEDAHVWEDNWDDDNVEDDFSNQLRA |
| | TVLLMVLVCETPYGCYVLHQKERMCSAFLCC |
| 68 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 69 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 70 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 71 | MSEKKQPVDLGLLEEDDEFEEFPAEDWAGLDEDEDAHVWEDNWDDDNVEDDFSNQLRA |
| | TVLLMKKVYETPYGCYILHQKGRMCSAFLCC |
| 72 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 73 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 74 | MSEKKQPVDLGLLEEDDEFEEFPAEDWAGLDEDEDAHVWEDNWDDDNVEDDFSNQLRA |
| | TVLLMIKVYETPYGCYILHQKGRMCSAFLCC |
| 75 | SEKKQPVDLGLLEEDDEFEEFPAEDWAGLDEDEDAHVWEDNWDDDNVEDDFSNQLRAT |
| | VLLMILVAETPYGAYV |
| 76 | SEFEDANKEQGEVPNDDVSDELPKLLDDDEDELRGALFFQWHDWEEAEDNDWDVEDA |
| 77 | SELDFWDNWKDRQADLPAASDDEDFENDLAEVDHEDPLVDGNEWDGDEVEFELEEQK |
| 78 | SDADEFDDDNSVWGWEDDEVEPNDAFQPLGDVRANEELDEKDHKLDEEADLQWEFEL |
| 79 | SQWAPLDHLESDEWGAFEDEDFNNGDVEELDPEERDDEDLFQKDVLVAKADDEEWDN |
| 80 | STKKQPVDLGLLETDSEFTEFPATSWAGLSESESAHVWTSNWDSSNVTSDFSNQLRA |
| 81 | STKKQPVDLGLLETDDEFEEFPATSWAGLSEDESAHVWESNWDDSNVTSDFSNQLRA |
| 82 | STKKQPVDLGLLEEDDEFEEFPAESWAGLDEDESAHVWEDNWDDDNVTSDFSNQLRA |
| 83 | SEKKQPVDLGLLEEDDEFEEFPAEDWAGLDEDESAHVWEDNWDDDNVTSDFSNQLRA |
| 84 | GLAALALMVRQPAFALEIYMGVKHTALYVST |
| 85 | SLLAVFPTIEAARYKMGLLYHAVAATQGMLV |
| 86 | SEFEDANKEQGEVPNDDVSDELPKLLDDDEDELRGALFFQWHDWEEAEDNDWDVEDAT |
| | VLLMILVAETPYGAYVLHQKGRMASAFLAA |
| 87 | SELDFWDNWKDRQADLPAASDDEDFENDLAEVDHEDPLVDGNEWDGDEVEFELEEQKT |
| | VLLMILVAETPYGAYVLHQKGRMASAFLAA |
| 88 | SDADEFDDDNSVWGWEDDEVEPNDAFQPLGDVRANEELDEKDHKLDEEADLQWEFELT |
| | VLLMILVAETPYGAYVLHQKGRMASAFLAA |
| 89 | SQWAPLDHLESDEWGAFEDEDFNNGDVEELDPEERDDEDLFQKDVLVAKADDEEWDNT |
| | VLLMILVAETPYGAYVLHQKGRMASAFLAA |
| 90 | STKKQPVDLGLLETDSEFTEFPATSWAGLSESESAHVWTSNWDSSNVTSDFSNQLRATVL |
| | LMILVAETPYGAYVLHQKGRMASAFLAA |
| 91 | STKKQPVDLGLLETDDEFEEFPATSWAGLSEDESAHVWESNWDDSNVTSDFSNQLRATVL |
| | LMILVAETPYGAYVLHQKGRMASAFLAA |
| 92 | STKKQPVDLGLLEEDDEFEEFPAESWAGLDEDESAHVWEDNWDDDNVTSDFSNQLRATV |
| | LLMILVAETPYGAYVLHQKGRMASAFLAA |
| 93 | SEKKQPVDLGLLEEDDEFEEFPAEDWAGLDEDESAHVWEDNWDDDNVTSDFSNQLRAT |
| | VLLMILVAETPYGAYVLHQKGRMASAFLAA |
| 94 | SEKKQPVDLGLLEEDDEFEEFPAEDWAGLDEDEDAHVWEDNWDDDNVEDDFSNQLRAG |
| | LAALALMVRQPAFALEIYMGVKHTALYVST |
| 95 | SEKKQPVDLGLLEEDDEFEEFPAEDWAGLDEDEDAHVWEDNWDDDNVEDDFSNQLRAS |
| | LLAVFPTIEAARYKMGLLYHAVAATQGMLV |

1. An artificial polypeptide of formula (I):
X—Y (I),
wherein X is a moiety comprising a sequence having at least 80% identity with SEQ ID NO. 1, and
Y is a moiety comprising 10 to 50 amino acids, at least 50% of which are selected from I, V, L, F, C, M, and A;
wherein Y comprises a total number of Cysteine (C) of less than 5.

2.-8. (canceled)

9. The artificial polypeptide of claim 1, wherein a total number of hydrophobic amino acids in Y is more than 8 or is no more than 5.

10.-13. (canceled)

14. The artificial polypeptide of claim 1, wherein Y comprises a sequence having at least 90% identity with any one selected from SEQ ID NOs. 3-15, 58-61 and 84-85 or wherein the artificial polypeptide comprises a sequence having at least 90% identity with any one selected from SEQ ID NOs. 29-41, 54-57 and 91-95.

15. (canceled)

16. (canceled)

17. An artificial polypeptide of formula (II):
X—Y (II),
wherein X is a moiety comprising a sequence having at least 80% identity with SEQ ID NO. 1, and
Y is a moiety comprising 10 to 50 amino acids, at least 50% of which are selected from I, V, L, F, C, M, and A;
wherein Y comprises a sequence having at most 90% identity with SEQ ID NO. 2.

18.-20. (canceled)

21. The artificial polypeptide of claim 17, wherein at least 50% amino acids of X are selected from R, K, N, D, Q, E, and H.

22.-24. (canceled)

25. The artificial polypeptide of claim 17, wherein a total number of hydrophobic amino acids in Y is more than 8 or is no more than 5.

26.-28. (canceled)

29. The artificial polypeptide of claim 17, wherein Y comprises a sequence having at least 90% identity with any one selected from SEQ ID NOs. 16-18 or wherein the artificial polypeptide comprises a sequence having at least 90% identity with SEQ ID NOs. 42-44.

30. (canceled)

31. (canceled)

32. An artificial polypeptide of formula (III):
X—Y (III),
wherein X is a moiety comprising 40 to 65 amino acids, at least 50% of which are selected from H, R, K, D, Q, N and E; and
Y is a moiety comprising a sequence having at least 80% identity with SEQ ID NO. 2,
wherein a total number of H, R, K, D, Q, N and E in X is less than 33.

33.-35. (canceled)

36. The artificial polypeptide of claim 32, wherein at least 50% amino acids of Y are selected from I, V, L, F, C, M, and A.

37.-39. (canceled)

40. The artificial polypeptide of claim 32, wherein a total number of hydrophilic amino acids in X is more than 10 or no more than 15.

41.-45. (canceled)

46. The artificial polypeptide of claim 32, wherein X comprises a sequence having at least 90% identity with any one selected from SEQ ID NOs. 23-27_or wherein the artificial polypeptide comprises a sequence having at least 90% identity with SEQ ID NOs. 49-53.

47. (canceled)

48. (canceled)

49. An artificial polypeptide of formula (IV):
X—Y (IV),
wherein X is a moiety comprising 40 to 65 amino acids, at least 50% of which are selected from H, R, K, D, Q, N and E; and
Y is a moiety comprising a sequence having at least 80% identity with SEQ ID NO. 2,
wherein X comprises a sequence having at most 90% identity with SEQ ID NO. 1.

50. The artificial polypeptide of claim 49, wherein X comprises a sequence having at most 80% identity with SEQ ID NO. 1.

51.-55. (canceled)

56. The artificial polypeptide of claim 49, wherein at least 50% amino acids of Y are selected from I, V, L, F, C, M, and A.

57.-62. (canceled)

63. The artificial polypeptide of claim 49, wherein X comprises a sequence having at least 90% identity with any one selected from SEQ ID NOs. 19-22 and 76-79.

64. (canceled)

65. The artificial polypeptide of claim 49, wherein the artificial polypeptide comprises a sequence having at least 90% identity with SEQ ID NOs. 45-48 and 86-89.

66. An artificial polypeptide of formula (V):
X—Y (V),
wherein X is a moiety comprising a sequence having at least 80% identity with SEQ ID NO. 1, and
Y is a moiety comprising 10 to 30 amino acids, wherein Y comprises a sequence having at least 10 continuous AAs of SEQ ID NO: 2.

67.-72. (canceled)

73. The artificial polypeptide of claim 66, wherein at least 50% amino acids of X are selected from R, K, N, D, Q, E, and H.

74. The artificial polypeptide of claim 66, wherein Y comprises a sequence having at most 80% identity with SEQ ID NO. 2, or wherein the artificial polypeptide comprises a sequence having at least 90% identity with any of SEQ ID NOs. 53-56.

75.-77. (canceled)

78. An artificial polypeptide of formula (VI):
X—Y (VI),
wherein X is a moiety comprising a mutant of the sequence SEQ ID NO. 1, characterized in that the mutant has at least 1, 2, 3, 4, 5, 6, 7, 8, or 9 D in SEQ ID NO. 1 mutated to S, and/or at least 1, 2, 3, 4, 5, or 6 E in SEQ ID NO. 1 mutated to T; and
Y is a moiety comprising a mutant of the sequence SEQ ID NO. 2, characterized in that the mutant has at least 1, 2, 3, 4, or 5 C in SEQ ID NO. 2 mutated to A.

79. The artificial polypeptide of claim 78, wherein X comprises a sequence having at least 70% identity with SEQ ID NO. 1.

80. (canceled)

81. The artificial polypeptide of claim 78, wherein Y comprises a sequence having at least 80% identity with SEQ ID NO. 2.

82. (canceled)

83. The artificial polypeptide of claim 78, wherein at least 50% amino acids of Y are selected from I, V, L, F, C, M, and A.

84. The artificial polypeptide of claim 78, wherein a total number of R, K, T, A, N, Q, D, E, S, and G in X is more than 30.

85. The artificial polypeptide of claim 78, wherein a total number of W, Y, F, M, L, I, and V in X is no more than 20.

86. (canceled)

87. (canceled)

88. The artificial polypeptide of claim 78, wherein X comprises a sequence having at least 80% identity with any one selected from SEQ ID NOs. 80-83, or wherein Y comprises a sequence having at least 80% identity with SEQ ID NO. 3.

89.-93. (canceled)

94. The artificial polypeptide of claim 78, wherein the artificial polypeptide comprises a sequence having at least 80% identity with any one selected from SEQ ID NOs. 90-93.

95. (canceled)

96. (canceled)

97. A mutant of artificial polypeptide of formula (I),
X—Y (I),
wherein X is a moiety comprising a sequence having at least 80% identity with SEQ ID NO. 1,
Y is a moiety comprising 10 to 50 amino acids, at least 50% of which are selected from I, V, L, F, C, M, and A,
Y comprises a total number of Cysteine (C) of less than 5,
characterized in that the mutant has at least 1, 2, 3, 4, 5, 6, 7, 8, or 9 D in X mutated to S, and/or at least 1, 2, 3, 4, 5, or 6 E in X mutated to T.

98.-169. (canceled)

170. An ophthalmic formulation for treating or preventing dry eye (DE) or DE associated disorders, stimulating tears, stabilizing tear film or any combination thereof, wherein the ophthalmic formulation comprises an artificial polypeptide of claim 1, or a polypeptide having at least 80% identity with any one selected from SEQ ID NO, 29-57, 62-74, and 91-95 or a fragment or variant thereof.

171.-178. (canceled)

179. A method for treating or preventing dry eye (DE) or DE associated disorders, stimulating tears, stabilizing tear film or any combination thereof in a subject in need thereof, comprising administering to the subject an effective amount of an artificial polypeptide of claim 1, or a polypeptide having at least 80% identity with any one selected from SEQ ID NO. 29-57, 62-74, and 91-95 or a fragment or variant thereof.

180.-184. (canceled)

185. A contact lenses care product, wherein the contact lenses care product comprises an artificial polypeptide of claim 1, or a polypeptide having at least 80% identity with any one selected from SEQ ID NO. 29-57, 62-74, and 91-95 or a fragment or variant thereof.

186.-191. (canceled)

192. A method for preparing a contact lenses care product, comprising the step of combining an artificial polypeptide of claim 1, or a polypeptide having at least 80% identity with any one selected from SEQ ID NO. 29-57, 62-74, and 91-95 or a fragment or variant thereof with one or more acceptable ingredients for contact lenses care product.

193.-204. (canceled)
